(19) United States
(12) Patent Application Publication
(10) Pub. No.: US 2026/0272749 A1
(43) Pub. Date: Sep. 17, 2026

(54) Method and device for pressure therapy

(71) Applicant: BTL Medical Development a.s., Prague 6 (CZ)

(72) Inventors: Tomáš Schwarz, Praha 6 (CZ); Ondrej Prouza, Rícany (CZ); Stanislav Kubín, Praha 8 (CZ)

(21) Appl. No.: 19/457,741

(22) Filed: Jan. 23, 2026

Related U.S. Application Data
(63) Continuation of application No. PCT/IB2025/055227, filed on May 20, 2025.
(60) Provisional application No. 63/649,650, filed on May 20, 2024.

(51) Int. Cl. A61G 10/02; A61H 9/00; A61M 1/00

(52) U.S. Cl. CPC A61G 10/023; A61H 9/005; A61M 1/90; A61G 2203/34

(57) ABSTRACT

The present invention relates to methods and apparatus for providing a pressure therapy.

### CROSS-REFERENCE TO RELATED APPLICATIONS

This application is a continuation of PCT International Application No. PCT/IB2025/055227, filed on May 20, 2025, which claims priority to U.S. Provisional Patent Application No. 63/649,650 filed on May 20, 2024, each of which is incorporated herein by reference in its entirety.

### FIELD OF THE INVENTION

The present invention relates to methods and apparatus for providing a pressure therapy.

### BACKGROUND

The present invention is in the domain of medical devices and therapeutic methodologies, specifically relating to pressure therapy, e.g. vacuum compression therapy, also known as vacuum pressure therapy. This system is designed to stimulate blood flow and optimize wound healing and tissue repair processes by leveraging the principles of negative pressure therapy or positive pressure therapy. It may be used in medical, physiotherapy, or aesthetic treatments. The invention introduces a novel approach to managing various diseases affecting blood flow, wounds, and other medical, dermatological, and/or aesthetic conditions, providing a versatile and efficient solution for healthcare practitioners and subjects alike.

The present invention also relates to pressure-controlled environments and, more specifically, to systems and methods for managing both hyperbaric and hypobaric conditions within chambers. Hyperbaric chambers provide elevated atmospheric pressure, while hypobaric chambers simulate reduced atmospheric pressure. The invention encompasses a versatile and adaptable approach to modulating pressure environments for various applications, including medical treatments, physiological treatments, and aesthetic treatments.

Hyperbaric therapy may be used to perform various treatments and actions in a positive-pressure environment. A popular type of hyperbaric therapy is hyperbaric oxygen therapy, which involves a subject breathing oxygen in an environment with increased pressure. It is widely used to treat decompression sickness (occurring during scuba diving or ascent to altitude), air bubbles in the blood vessels, various infections, chronic wounds, and others.

Hypobaric therapy may be used to perform various treatments and actions in a negative-pressure environment. A popular type of hypobaric therapy is negative pressure wound therapy, a medical technique used for the management of wounds. Its goal is to promote wound healing by creating an environment that facilitates the removal of excess fluids, stimulates blood flow, and helps reduce edema and/or swelling.

The alternating negative and positive pressure of vacuum compression therapy (pressure therapy) contributes to improved blood circulation, making it an effective tool for enhancing blood flow to limbs. This application is particularly valuable in addressing circulatory issues and promoting vascular health. Vacuum compression therapy may be used to treat various ischemic conditions (such as ischemia), where there is a restriction in blood supply to the tissues, causing a shortage of oxygen, glucose and nutrients needed for cellular metabolism. Vacuum compression therapy can improve perfusion and oxygenation of the tissues and can enhance venous return. Vacuum compression therapy finds utility in physiotherapy by assisting in the management of musculoskeletal conditions.

Controlled pressure variations in vacuum compression therapy (pressure therapy) may aid in reducing inflammation, promoting tissue recovery, and enhancing overall rehabilitation outcomes. In aesthetic applications, the therapy may be employed for body contouring, cellulite reduction, and skin rejuvenation. The precise control of pressure facilitates targeted therapy, promoting collagen synthesis and improving the overall appearance of the skin. In wound treatment applications, the controlled pressure variations may be used to treat chronic wounds (formed, for example, because of diabetes or radiation injury), surgical incisions, and other small-area or large-area wound sites. The device may be used in combination with various wound dressings to create a seal around the wound and to remove excess fluids from the wound, creating a cleaner wound environment. Removing excess fluid may also contribute to the reduction of edema and/or swelling around the wound. The stimulation of the blood flow may also be beneficial for tissue repair and may optimize wound healing.

### SUMMARY OF THE INVENTION

The present invention introduces a groundbreaking advancement in the field of medical devices, specifically in the area of vacuum compression therapy (pressure therapy). The system is ingeniously designed to address and surpass the limitations of traditional compression therapy methods, offering an innovative solution for a wide range of medical and aesthetic applications.

This invention comprises a novel and versatile vacuum compression therapy (pressure therapy) system designed to optimize blood flow, promote wound healing, and facilitate tissue repair through the application of controlled pressure variations. This innovative system encompasses both positive and negative pressure modalities, allowing for a comprehensive approach to address a wide range of medical, physiological, and aesthetic conditions. The invention may be used to treat various problems that affect blood flow, like, for example, a diabetic foot, ischemia and circulatory and peripheral arterial diseases.

In one aspect, the vacuum compression therapy (pressure therapy) system may be configured to provide positive and negative pressure modalities. In another aspect the vacuum compression therapy (pressure therapy) system may be configured to provide only positive pressure modalities. In another aspect the vacuum compression therapy system (pressure therapy) may be configured to provide only negative pressure modalities. In yet another aspect, the vacuum compression therapy (pressure therapy) system may be configured to provide, in addition to positive and/or negative pressure modalities, at least one phase or portion of therapy during which the pressure is maintained at or near zero pressure, i.e., atmospheric or ambient pressure.

This invention may also be used to treat carpal tunnel syndrome and various traumatic conditions, neuropathies, and syndromes. The invention may be used, for example, to treat reflex sympathetic dystrophy syndrome. The invention may be used to quicken the healing of wounds or ulcers, venous ulcers, stasis ulcers, and/or varicose ulcers. The invention may be used in sports medicine to, for example, relieve sore muscles. The invention may be used in aesthetics to, for example, treat cellulite or provide tightening of the skin.

At the heart of this invention is an adjustable sealing mechanism, meticulously developed to accommodate a variety of body parts. This mechanism is a pivotal feature of the system, ensuring a secure and comfortable fit for the subject. By providing an airtight seal, it enhances the overall effectiveness of the therapy, ensuring that the therapeutic benefits are maximized. This adaptability is crucial for treating different parts of the body, making the system versatile for applications ranging from limb therapy in medical rehabilitation to body contouring in aesthetic treatments.

Another key aspect of this advanced system is its capability to produce very fast pressure changes. This functionality broadens the scope of therapy, allowing for a diverse range of applications. The system can swiftly transition between different pressure levels, making it suitable for various therapy protocols. This rapid modulation is particularly beneficial in scenarios where alternating between positive and negative pressure is required, such as in promoting blood circulation, reducing inflammation, and accelerating wound healing. The ability to quickly adjust pressure settings enables tailored therapies, catering to the specific therapeutic needs of individual subjects.

In another aspect, the therapy chamber's capacity for rotation adds another layer of functionality to the system. This feature allows the therapy chamber to be oriented in multiple directions, facilitating targeted therapy for specific areas. It also allows for the therapy chamber to accommodate different subject positions, enhancing comfort and effectiveness. This flexibility is especially advantageous in providing comprehensive therapy coverage and ensuring that therapy is delivered precisely where it is most needed.

In accordance with another aspect of the invention, a therapy unit to treat a body part of a subject (e.g. patient) is provided.

As used herein, the terms ‘subject’ and ‘patient’ are used interchangeably and refer to any animal, including a human, to whom a treatment, diagnosis, or observation is directed.

In accordance with yet another aspect of the invention, a therapy unit comprising a sealing mechanism to seal a body part is provided.

In accordance with another aspect of the invention, a system for delivering pressure changes to a body part of a subject is provided.

The aspects of the invention may be combined.

The treated body part may comprise limb, arm, hand, leg, feet, trunk, thigh, lower part of the body, or others.

In one aspect of the invention, the device may be used for therapy of various aesthetic conditions, such as, for example, treatment of cellulite, skin rejuvenation, tightening and firming of the skin, improving skin texture and tone, body sculpting, strengthening of the connective tissue, body contouring, improvement of the body circulation, enhanced lymphatic drainage, reduction of stretch marks, reduction of scars, reduction of subcutaneous fat tissue, or muscle toning.

In summary, the presented aspects of the vacuum compression therapy (pressure therapy) system represent a significant leap forward from traditional methods. Its adjustable sealing mechanism not only ensures subject comfort but also enhances the efficiency of therapy delivery. The capability for rapid pressure changes opens up a wide spectrum of therapeutic possibilities, allowing for customized, subject-specific treatment plans. The rotatable therapy chamber further elevates the system's adaptability and accessibility, making it suitable for a diverse array of medical and aesthetic treatments. With these innovative features, the system stands out as a versatile, efficient, and subject-friendly solution in the realm of compression therapy.

### BRIEF DESCRIPTION OF THE FIGURES

FIG. 1A is a schematic diagram of a proposed system.

FIG. 1B is a schematic diagram of a proposed system.

FIG. 2 shows one exemplary schematic diagram of the therapy unit.

FIG. 3A-D are schematic diagrams of the therapy chamber.

FIG. 4A-C are schematic diagrams of the sealing unit.

FIG. 5A-B are schematic diagrams of the sealing unit.

FIG. 6A-B are schematic diagrams of the sealing unit.

FIG. 7 is a schematic diagram of the device.

FIG. 8A-B are schematic diagrams of the therapy chamber comprising at least two openings.

FIG. 9 shows one aspect of the device according to the invention.

FIG. 10 shows one aspect of the device according to the invention comprising two parts, the upper part and the bottom part.

FIG. 11 shows one aspect of the device according to the invention comprising three parts, the upper part, middle part and bottom part.

FIG. 12 shows a front view of a first part of the click mechanism comprising the cuff equipped with a first part of a cuff connector and a closing member.

FIG. 13 shows a back view of a first part of the click mechanism comprising the cuff equipped with a first part of a cuff connector and two closing members.

FIG. 14 shows a view of the mechanics of a second part of click mechanism without the inserted cuff.

FIG. 15 shows a view of the mechanics of both parts of the click mechanism connected together.

FIG. 16 shows the sealing unit comprising a moldable membrane.

FIG. 17 shows the moldable membrane.

FIG. 18 shows a cross-sectional view of the moldable membrane.

FIG. 19 shows the sealing unit comprising a folded cuff.

FIG. 20 shows the sealing unit comprising two folded cuffs.

### DETAILED DESCRIPTION

Vacuum compression (pressure) therapy encompasses both positive and/or negative pressure modalities. It may aid in reducing inflammation, promoting tissue recovery, and enhancing overall rehabilitation outcomes. It can also be used for various aesthetic treatments, such as body contouring, cellulite reduction, and skin rejuvenation.

According to one aspect of the invention, one or more therapy units may be used to deliver pressure changes to the subject's body part. The device may be used to treat various body parts, including limbs, arms, hands, legs, feet, trunk or thighs, abdomen, torso, lower body part, genital tissue (e.g. vaginal tissue, penile tissue), head, neck or others.

In another aspect the invention may be used to treat multiple body regions simultaneously or sequentially for at least part of the duration of a therapy. In one non-limiting example, the proposed device may comprise one therapy chamber for therapy of two body parts (e.g. two limbs). In another non-limiting example, the proposed device may comprise two therapy chambers for therapy of two body parts (e.g. two limbs), wherein a first therapy chamber is used for therapy of a first body part and a second therapy chamber is used for therapy of a second body part. In another non-limiting example, the therapy chamber may contain almost the whole body of the subject except some parts, e.g. except head, hands, or feet.

In yet another aspect the invention may be used to treat the whole body of the subject.

According to some aspects of the invention, vacuum pressure therapy may be applied in combination with various treatments, for example, thermal treatment (e.g. heating of the body part, or cooling of the body part).

According to one aspect of the invention, vacuum pressure therapy may be applied in combination with a drug or solution. The drugs or solutions may be administered prior to the therapy, during the therapy or post-therapy.

In one aspect, drugs or solutions (e.g. hydrational, nutritional, pain relieving or cosmetic) may be administered on the body part and the vacuum pressure therapy may be applied in order to better absorb the drugs or solutions by the body part.

The therapy may have a duration range from 1 minute to 180 minutes, or from 1 minute to 60 minutes, or from 5 minutes to 60 minutes, or from 10 minutes to 45 minutes or, from 15 minutes to 30 minutes, or from 1 minute to 10 minutes, or from 10 minutes to 20 minutes, or from 15 minutes to 45 minutes, or from 20 minutes to 40 minutes.

Disclosed methods and devices may be used for various aesthetic and cosmetic therapies including but not limited to treatment of dermatitis, atrophic disorders (e.g. stretch marks), wrinkles, dimpled skin, hypertrophic disorders (e.g. scars), treatment of varicose veins, treatment of spider veins, circumferential reduction, weight reduction, body shaping, core shaping, skin tightening, skin rejuvenation, skin toning, treatment of cellulite tissue, improvement of skin elasticity, enhancement of skin hydration, treatment of bones, induction of bone regeneration, treatment of adipose tissue, removal of adipose tissue, adipose tissue catabolismshrinking of adipose tissue, muscle growth, muscle treatment, denaturation of collagen, denaturation of elastin, deposition of nascent collagen, deposition of nascent elastin, breast tightening, volume reduction of abdomen, volume reduction of thigh, and/or volume reduction of love handles. The methods and devices may also be used for rehabilitation, palliative care, improvement of oxygenation, increase of metabolism, improvement of ion transport, improvement of microcirculation, treatment of diabetes, wound healing, improvement of blood flow, pain relief and/or enhancement of lymphatic drainage. Treatment may include contraction, relaxation, apoptosis, necrosis, cavitation and/or massage.

The disclosed devices and related methods of therapy may include application of a pressure gradient by one or more pressure generators. The disclosed devices and related methods of therapy may include application of a pressure gradient by one or more pressure generators together with the application of at least one additional type of energy.

The pressure gradient may be induced by a hyperbaric and/or hypobaric environment inside a therapy device or inside a part of the therapy device (e.g. therapy chamber). The pressure gradient may include at least one hypobaric period when the pressure inside the device or inside a part of the therapy device (e.g. therapy chamber) may be below atmospheric pressure and/or at least one hyperbaric period when the pressure inside the device or inside a part of the therapy device (e.g. therapy chamber) may be above the atmospheric pressure. Atmospheric pressure may be applied during an atmospheric pressure period. Atmospheric pressure may refer to the pressure outside of the device and/or a standard pressure defined as 1 bar (equal to 100 kPa) or 1 atm (101,325 Pa) or 101.3 kPa or 101.33 kPa or 101.6 kPa or 101 kPa or 99.99 kPa.

In one aspect of the invention, the pressure gradient in the therapy unit 105 may comprise at least two consecutive hyperbaric phases and/or at least two consecutive hypobaric phases. The number of consecutive hyperbaric phases or consecutive hypobaric phases may be in the range of 2 to 100, or in the range of 2 to 50, or in the range of 2 to 40, or in the range of 2 to 30, or in the range of 2 to 20, or in the range of 2 to 10, or in the range of 2 to 6.

The at least one phase with atmospheric pressure may be present between at least two consecutive hyperbaric phases and/or at least two consecutive hypobaric phases, or at least one phase with atmospheric pressure may be present between a plurality of consecutive hyperbaric phases and a plurality of consecutive hypobaric phases.

In one aspect, the at least one phase at atmospheric pressure may be present between at least two consecutive pressure phases, including between two hyperbaric phases, two hypobaric phases, or between a hyperbaric and a hypobaric phase, regardless of the order.

The pressure in the therapy unit 105 may be changed during the hyperbaric phase and/or the hypobaric phase.

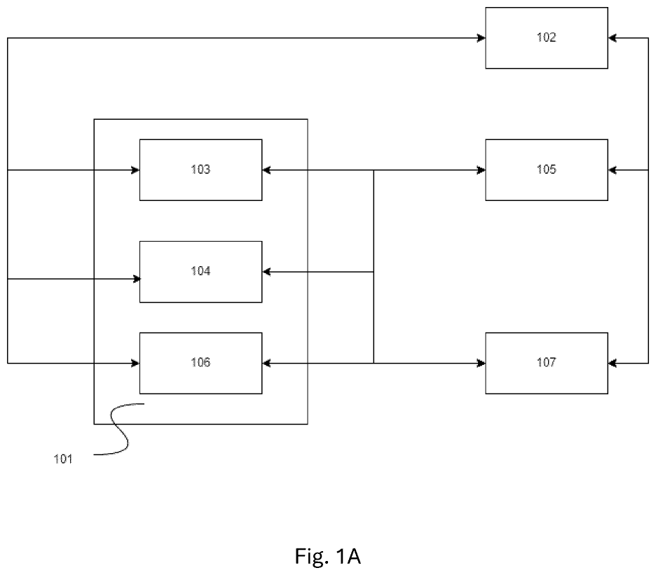

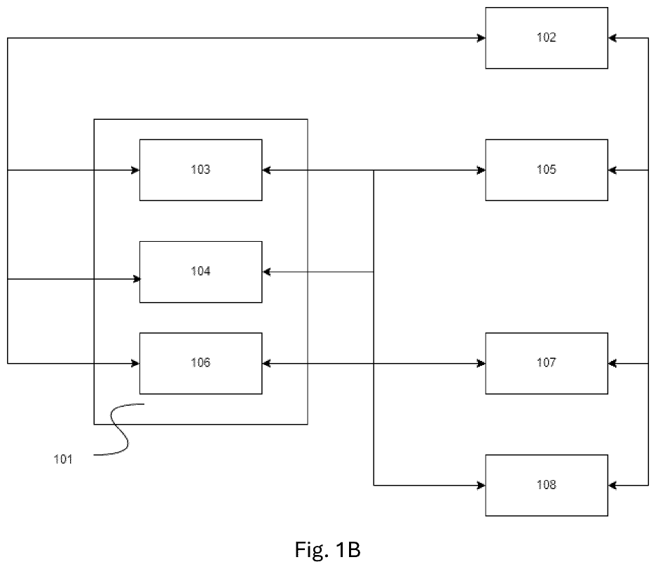

FIG. 1A shows one exemplary schematic diagram of a proposed system (device). The system may comprise a main unit 101, a power supply 102, a control unit 103, a user interface 104, a therapy unit 105, a pressure generator 106, and at least one sensor 107. The main unit 101 may comprise the control unit 103, the user interface 104, and/or the pressure generator 106. The power supply 102 may be a part of the main unit 101 or the power supply 102 may be located outside the main unit 101.

In one aspect of the invention the device for pressure (positive and/or negative) therapy may comprise a main unit 101 and a therapy unit 105. The therapy unit 105 may comprise a sealing unit 202. The main unit 101 may comprise a base 115, a stand 150, an interface holder 111 and a user interface 104 coupled to the interface holder 111, as shown in FIG. 9. In one aspect, the therapy unit 105 may be coupled to a stand 150 and the stand 150 may be coupled to the base 115.

In one aspect, the interface holder 111 may be coupled to the stand 150 and positioned above the therapy unit 105, as shown in FIG. 9. In another aspect, the interface holder 111 may be coupled to the stand 150 and positioned sideways around the therapy unit 105. In another aspect, the interface holder 111 may be coupled to the stand 150 and positioned at least partially under the therapy unit 105. In another aspect, the interface holder 111 may be coupled to the base 115.

The power supply 102 may include a standard power cord, power plug and/or battery.

The user interface 104 may include a notebook, PC, mobile phone, tablet and/or control panel including a display, buttons or other control member. A display on a control panel may be responsive to touch. A display may be part of the user interface 104 providing set-up of the therapy, providing results of imaging and/or sensing the conditions and/or changes of the conditions of the tissue. Optionally, the display may also be configured to visualize information about the therapy, such as the length of the therapy, the amount of transferred energy, data from sensors, etc. An operator and/or a subject may set one or more therapy parameters related to tissue and/or pressure and/or a pressure gradient.

The vacuum compression therapy (pressure therapy) device according to the invention may comprise a light signaling. The light signaling may indicate the status of the therapy, may provide visual cues, feedback, or error signaling, or any other appropriate or requested status.

The light signaling may comprise changes in color, for example red for error or stop, green for operational, or any other appropriate light signaling color.

The running therapy may be signaled by a blue color.

The light signaling may comprise an LED (Light Emitting Diode), e.g. LED strip, a display, OLED (Organic Light Emitting Diode) display, LCD displays (Liquid Crystal Display), signal light, fiber optic light guides, laser diodes, electroluminescent panels (EL Panels), light bars or strips, or any other appropriate light signaling component.

LEDs have low power consumption, long lifespan, and are available in various colors and brightness levels. LEDs are compact and easy to integrate. LEDs may be provided as single-color LEDs, for example in red, green, or blue for status indication, or multi-color LEDs (RGB or RGBA), that combine colors to indicate different states. LCD Displays (Liquid Crystal Displays) may provide a visual feedback with text and graphics.

Light signaling, e.g. LED strip signaling, may indicate for example the course of therapy, movement of the therapy unit, current pressure in the therapy unit, e.g. hyperbaric or hypobaric phases of the therapy.

In one aspect of the invention, the light signaling may be placed around the entrance to the therapy unit. The light signaling, e.g. LED strip, may signalize or indicate a direction, where to move the therapy unit in order to get the proper position for the therapy.

The power supply 102 may communicate with the control unit 103 and the user interface 104. The pressure generator 106 may be controlled by the control unit 103. The control unit 103 may also evaluate feedback from at least one sensor 107 and adjust the therapy parameters accordingly.

The pressure generator 106 may be connected to at least one of: the therapy unit 105, the sealing unit 202 or the inflatable seal placed in the opening 204 in the therapy unit 105 in order to provide the requested pressure.

In one aspect of the invention, one pressure generator 106 may be connected to the therapy unit 105 and to the sealing unit 202 in order to provide the requested pressure.

In other aspect of the invention, one pressure generator 106 may be connected to the therapy unit 105, to the sealing unit 202 and to the inflatable seal in the opening 204 in the therapy unit 105 in order to provide the requested pressure.

In another aspect of the invention, the therapy unit 105 and the sealing unit 202 may be connected to two separate pressure generators 106.

In another aspect of the invention, the therapy unit 105, the sealing unit 202 and the inflatable seal in the opening 204 in the therapy unit 105 may be connected to three separate pressure generators 106.

The control unit 103 may comprise a printed circuit board (PCB) including a microprocessor and memory for storing therapy data related to each subject and/or therapy. The control unit 103 may provide therapy control by stabilization of one or more therapy parameters related to tissue and/or pressure gradient. Parameters may be measured by at least one sensor 107, as described below.

The control unit 103 may control the therapy parameters such as therapy duration, therapy pressure (e.g. pressure difference between the pressure in the therapy chamber and the atmospheric pressure), duration of the pressure pulses, therapy pattern, the flow of air, the amount of heating delivered to the tissue, the amount of cooling delivered to the tissue, position of the therapy unit, pulse mode, and/or others. The therapy parameters may be chosen from a predetermined group of protocols or may be set by the user. The therapy parameters may be chosen from a range of safe thresholds. The therapy parameters may be adjusted by the user or automatically by the control unit 103 during the therapy. The therapy parameters may be adjusted by the control unit 103 based on the feedback measurements from at least one sensor 107. If the chosen therapy parameters are safe, the therapy may start.

The pressure generator 106 may create a pressure gradient, wherein the pressure gradient may be defined as a different pressure in the chamber (e.g. in the vicinity of the tissue) from the atmospheric pressure. The pressure generator 106 may include a blower, compressor, syringe, fan and/or pump. The pressure gradient may be executed by the transfer of a fluid, e.g. a gas or a liquid.

The rotation rate of the pressure generator 106 may be up to 4000 rotations per minute. The power output of a motor of the pressure generator 106 may be up to 5 kW. An operating voltage of the pressure generator 106 may be up to 600 V. The operating efficiency of the pressure generator 106 may be in the range of 30% to 99%. The magnitude of noise emitted by the pressure generator 106 may be from 5 dB up to 110 dB, wherein sound encapsulation of the device may provide a 5% to 90% decrease in noise.

The therapy unit 105 may comprise a therapy chamber that surrounds the body part. The therapy chamber may be connected to a pressure generator 106 that provides negative and/or positive pressure to the therapy unit 105. The therapy chamber may be configured such that the body part may be inserted into the therapy chamber through an entrance of the therapy chamber.

The therapy unit 105 may enclose the body part of the subject receiving therapy and seal the body part using a sealing mechanism. The sealing mechanism may be flexible or rigid or partially flexible (e.g. some parts may be flexible and some parts may be rigid). The sealing mechanism may be made of rubber, silicone, latex, polymer, plastic, and others. The sealing mechanism may be detachable from the other parts of the device (e.g. from the therapy chamber).

In one aspect, the sealing mechanism may comprise an inflatable part. The sealing mechanism may seal the entrance to the therapy chamber by inflation of the sealing mechanism, e.g. by inflation of the inflatable part of the sealing mechanism.

In another aspect, the sealing mechanism may use traction to open the entrance to the therapy chamber before the body part is inserted into the therapy chamber, and then after the body part is inserted into the therapy chamber, the traction is released, and the body part is securely sealed in the therapy chamber.

In another aspect, the sealing mechanism may be mechanically powered to seal the entrance to the therapy unit 105. The sealing mechanism may be powered by a motor, by a device based on hydraulic power, and/or mechanically, e.g. by hand.

The pressure difference between the pressure in the therapy chamber and the atmospheric pressure may be in a range from 0.001 kPa to 1000 kPa, from 0.01 kPa to 1000 kPa, from 0.01 kPa to 100 Pa, or from 0.1 kPa to 100 kPa, or from 1 kPa to 100 kPa, or from 1 kPa to 20 kPa, or from 10 kPa to 50 kPa, or from 5 kPa to 20 kPa, or from 10 kPa to 15 kPa, or from 20 kPa to 30 kPa.

The device may comprise at least one sensor 107. At least one sensor 107 may include a pressure sensor, a flow sensor, a force sensor, a rotation sensor, a turbine sensor, a sensor for gas monitoring, a sensor for liquid monitoring, a humidity sensor, an oxygenation sensor, a saturation sensor, a sensor of color, a thermal camera, a temperature sensor, a sensor for measuring the volume of the body part (e.g. limb) and its change, a sensor for measuring oxygenation, a sensor for measuring blood saturation, a sensor for measuring the perfusion, a sensor for measuring the distance of the therapy chamber from the subject's surface, a sensor detecting movement, a voltage sensor, a current sensor, an impedance sensor, a gyroscope, a biochemical sensor, a biosensor, a sensor detecting velocity, a sensor detecting movements, a sensor measuring one or more of permittivity, conductivity, inductance, or capacitance, a sensor measuring a value of the electromagnetic field, or a sensor for measuring the contact between the sealing mechanism and a body part of the subject. At least one sensor 107 may be used in an invasive or non-invasive manner. At least one sensor 107 may be positioned in the therapy unit 105, on the therapy unit 105, outside the therapy unit 105, or on the subject's body part to be treated, and at any other positions on the device and/or subject.

The saturation sensor may be used to measure the amount of oxygen-carrying hemoglobin in the blood relative to the amount of hemoglobin not carrying oxygen. The saturation sensor may be used to indicate the oxygen levels in the body part, prior to, during and/or post-therapy. The saturation levels may be used to monitor and/or evaluate the course of the therapy. The saturation sensor may be in communication with the control unit 103.

The temperature of the tissue may be monitored using one or more temperature sensors. The temperature sensor can be a thermocouple, a resistance thermometer, a thermometer, a thermostat, an optical sensor, or an infrared thermometer. One or more temperature sensors may be used to measure the temperature of the treated tissue, the skin surface, and/or the therapy unit 105.

The temperature may be evaluated indirectly. Indirect measurement of temperature may be a measure of a different physical parameter other than temperature. Such parameters can, for example, be the impedance or resistivity of the tissue and/or others.

The color and/or temperature of a subject's body part may be measured differentially. The color and/or temperature may be measured before the therapy starts, and measurements may be acquired during therapy. The measurements before the therapy started and during the therapy may be compared and evaluated (e.g. by the control unit). The color and/or temperature measurements may be used to monitor and/or evaluate the course of the therapy. The color sensor and/or temperature sensor may be in communication with the control unit 103.

The thermal camera may be positioned inside the therapy unit 105 and/or outside the therapy unit 105. The thermal camera may be used to monitor changes in the temperature of the body part, and to monitor the temperature distribution of the body part.

The at least one temperature sensor may be positioned inside the therapy unit 105 and may be used to monitor the temperature inside the therapy unit 105.

At least one therapy parameter may be adjusted based on the data provided by at least one temperature sensor. The at least one therapy parameter may be for example the therapy course, temperature inside therapy unit 105, the time, effect on a blood flow, or any other appropriate parameter.

The volume of the patient's body part, e.g. limb, may be measured by at least one sensor 107, for example by a sensor for measuring the volume of the body part (e.g limb).

In one aspect, the volume of the patient's body part (e.g limb) may be measured based on the principle of conductivity. This method relies on the fact that electrical conductivity of tissues changes with the amount of fluid, e.g. blood, in the body part. Blood and interstitial fluid conduct electricity well, while fat and bone do not. So, by measuring electrical impedance across a body part, changes in volume may be determined. A small, safe alternating current (AC) may pass through the body part using surface electrodes. Other electrodes measure the resulting voltage drop, from which impedance (Z) is calculated using Ohm's law:

$$Z = V / I , \qquad i )$$

ii) where Vis voltage and I is current.

Since impedance is inversely related to the cross-sectional area of the body part (and therefore volume), changes in impedance may indicate changes in volume.

The therapy unit 105 and/or the therapy chamber may be provided with a conductive material, wherein the body part is conductive. The conductive material may be transparent. The conductive material may comprise for example Indium-Tin-Oxide (ITO) or Carbon nanotubes (CNTs), or any other appropriate conductive material.

In another aspect, the volume of the patient's body part may be measured based on translucency or other optical principles, using for example a camera, or any other appropriate device. The method may use light transmission or reflection to assess the volume and structure of a body part. The amount of light absorbed, scattered, or transmitted depends on the tissue composition, blood volume, and skin thickness. More volume typically means more scattering/absorption, especially due to blood. A photoplethysmography (PPG) method may be used. The principle of the photoplethysmography is that an LED sends light into the skin, and a photodetector measures how much light is reflected or transmitted. Pulsatile changes in blood volume (e.g., heartbeat) cause changes in light absorption. The method is appropriate for relative volume changes, often in fingers, hands, or arms. A 3D optical scanner may also be used for measurement of the volume of patient's body part, for example using a laser or structured light to capture the external shape of the body part. A 3D model may be reconstructed and volume calculated.

In another aspect, the volume of the patient's body part may be measured based on capacitive principle, for example using one or more electrodes. This method is based on measuring the capacitance between two electrodes. The electrodes may be placed near or around the body part. Capacitance may depend on the distance between electrodes, the dielectric properties of the tissue (i.e., how well it stores electric charge), and/or the geometry and volume of the body part between the electrodes, as the body part swells or changes shape/volume. The capacitance may change due to a change in either the effective dielectric constant (related to water content, fat, muscle) or the cross-sectional area of the body part.

The measurement of the volume of a patient's body part based on the capacitive principle may be carried out by detecting a change in capacitance (e.g., from C₁ to C₂) resulting from either (i) a change in the distance between the patient's body part and the therapy chamber 401, or (ii) a change in the ratio of the surface areas of the patient's body part and the therapy chamber 401. Both of these changes are induced by variations in pressure within the therapy chamber 401. The therapy chamber 401 may serve as the first electrode, while the patient's body part may serve as the second electrode. For example, the distance between the patient's body part and the therapy chamber 401 may be greater during a therapy phase when the pressure is higher than atmospheric pressure, compared to a therapy phase when the pressure is lower than atmospheric pressure. Similarly, the effective surface area of the patient's body part may be reduced during a therapy phase with higher-than-atmospheric pressure, compared to a phase with lower than atmospheric pressure.

The basic formula for capacitance:

$$C = {ε}_{r} {ε}_{0} * A / D ,$$

where:
i) C is capacitance
ii) εᵣ is relative permittivity of the tissue
iii) ε₀ is vacuum permittivity
iv) A is area of electrode overlap (related to body part size)
v) D is distance between electrodes.

In the method of measuring the volume of the patient's body part based on the capacitive principle the electrodes may be placed around or near the body part. A small AC voltage may be applied, and the capacitance is measured.

The position of the therapy unit 105 may be monitored using a position sensor. A position sensor may be, for example, an accelerometer or gyroscope.

In one aspect of the invention, the therapy may be enabled or disabled based on the actual position of the therapy unit 105, that may comprise the therapy chamber. For example the therapy may be disabled, when the therapy unit 105 is in the parking position. The parking position of the therapy unit 105 may be for example when the therapy unit 105 is in the vertical position. The parking position, for example the vertical position, of the therapy unit 105 may be suitable for movement or transport of the therapy device.

The parking position of the therapy unit 105, when the therapy is disabled, may be vertical, or may be in a specific angle to the vertical position. The angle may be for example in the range of 0° to 30° from the vertical position, or in the range of 2° to 28°, or in the range of 5° to 25°, or in the range of 8° to 22°, or in the range of 10° to 20° from the vertical position.

In another aspect, the parking position of the therapy unit 105, when the therapy is disabled, may be at a specific angle to the horizontal position. The therapy may be disabled when the angle is higher than 10°, 12°, 15°, 18° or 20° from the horizontal position.

Enabling or disabling of the therapy in relation to the current position of the therapy unit 105 may be controlled by a limit switch.

The parking position of the therapy unit 105 may be adjusted for example by pressing a button and/or by touching the user interface, for example the control panel with display, by a person providing the therapy.

The flow sensor may be used to monitor the velocity and/or the volume of the gas on the input and/or on the output of the therapy chamber. The data from the flow sensor may be used to calibrate the system, to regulate the power of the pressure generator 106, to regulate the speed of the pressure generator 106, and/or to regulate the pressure in the therapy unit 105.

The pressure sensor may be located inside the therapy unit 105, or connected to the therapy unit 105. The data from the pressure sensor may be used to calibrate the system, to regulate the power of the pressure generator 106, to regulate the speed of the pressure generator 106, and/or to regulate the pressure in the therapy unit 105.

The pressure may be measured differentially. The pressure sensor may be a mechanical pressure sensor, an electrical pressure sensor, a piezoelectric pressure sensor, an optical pressure sensor, and others.

At least one sensor 107 may serve as feedback and may be in communication with the control unit 103. At least one sensor 107 may be used to improve the efficiency of the therapy and prevent any discomfort and health risks. The therapy parameters may be manually or automatically adjusted and optimized.

The at least one sensor 107 may be located anywhere inside the therapy unit 105 and/or outside the therapy unit 105. The therapy unit 105 may comprise at least two sensors 107. The at least one sensor 107 may provide feedback about the state of the therapy unit 105 and/or the state of the sealing of the body part.

FIG. 1B shows another exemplary schematic diagram of a proposed system. According to some aspects of the invention, the device may comprise a treatment element 108.

The device may further apply at least one additional type of energy by a treatment element 108. The energy may be applied to at least one tissue layer and/or volume of the tissue such as adipose tissue, connective tissue, epidermis, dermis, hypodermis, basement membrane, subcutis, muscle, lymph vessel, cartilage, tendons, superficial muscular aponeurotic system, fascia, ligaments (e.g. Cooper's ligament), sebaceous glands, blood and/or blood vessel. The energy may be applied to the abdomen, thighs, hip, breast, legs, arms, tissues of the perineal region, genital tissue, back and/or foot. Energy may be provided by direct contact, indirect contact and/or contactless energy delivery element. The energy may be applied in a continuous and/or pulsed manner.

In one aspect, the treatment element 108 may comprise a thermal treatment element. Thermal treatment induces beneficial responses that provide potential improvements in cardiovascular health. Thermal treatment may improve the local blood perfusion by inducing vasodilatation, when blood vessels in the subject's body dilate, allowing more blood to flow through them. This results in lowering blood pressure and better oxygenation of a specific organ or an entire system.

The thermal treatment may also reduce pain, as the increased temperature in the affected area may reduce the discomfort and increase muscle flexibility. Applying heat to the affected body part stimulates blood circulation, delivering oxygen and nutrients and promoting healing. It also helps remove lactic acid buildup, relaxing and soothing muscles. In another aspect, the heat may target adipose tissue of the body part in order to reduce fat in the body part, which may, for example, lead to cellulite reduction.

The vacuum compression therapy (pressure therapy) in combination with thermal treatment may be used for various aesthetic benefits, such as, for example, tightening and firming of the skin, improving skin texture and tone, body sculpting, cellulite reduction, post-liposuction care, buttock enhancement, strengthening of the connective tissue, body contouring, improvement of the body circulation, enhanced lymphatic drainage, muscle toning, muscle relaxation, increased blood flow, reducing pain, enhancing tissue healing, reducing inflammation and others.

The subject's body part may be heated to a temperature in a range from 38° C. to 100° C., or 50° C. to 80° C., or 55° C. to 75° C., or 57° C. to 72° C., or 60° C. to 70° C., or 37.5° C. to 50° C., or 38° C. to 48° C., or 40° C. to 45° C., or 41° C. to 43° C.

The temperature at the output of the thermal treatment element may range from 38° C. to 200° C., or from 38° C. to 150° C., or from 38° C. to 100° C., or from 38° C. to 80° C., or from 38° C. to 60° C., or from 38° C. to 50° C., or from 38° C. to 45° C., or from 45° C. to 100° C., or from 50° C. to 100° C.

The thermal treatment may be provided in a continuous or alternating mode. The body part may be heated to a preset temperature for a duration of time. The body part may be heated for the whole duration of the vacuum compression therapy (pressure therapy). The temperature of the heating may be constant during the time of vacuum compression therapy (pressure therapy) or the temperature may alternate. The body part may be heated in a first period to a preset temperature, and then the heating may stop in a second period. The period of heating and not-heating may alternate periodically.

The alternating heating may have a profile that characterizes the temperature changes. The profile may be a square profile, a triangle profile, a ramp profile, a descending ramp profile, an exponential profile, an exponential decay profile and others. The profile may comprise a ramp-up time, a constant time and a ramp-down time.

The first period of heating or the second period of non-heating may have a duration in a range from 0.001 seconds to 10000 seconds, or from 0.1 seconds to 5000 seconds, or from 1 second to 1000 seconds, or from 1 second to 500 seconds, or from 120 seconds to 1200 seconds, or from 120 seconds to 1000 seconds, or from 60 seconds to 600 seconds, or from 60 seconds to 360 seconds, or from 1 second to 120 seconds or from 1 second to 50 seconds or from 1 second to 20 seconds, or from 5 seconds to 15 seconds, or from 1 seconds to 10 seconds, or from 1 second to 5 seconds, or from 2 seconds to 3 seconds.

The thermal treatment element may comprise an object filled with heated fluid and/or a heated fluid circulation unit, one or more thermoelectric heaters, one or more Peltier elements, one or more heat sinks, one or more heat conductors, one or more electromagnetic energy radiating elements, one or more microwave energy generation elements, one or more radiofrequency generation elements, one or more light generation elements, and others. The heated fluid may be a substance comprising air, CO₂, O₂, nitric oxide, nitrogen, water, distilled water, deionized water, water-doped NaCl, ethanol, oil, and others.

The heated fluid may be a gas and may flow to the therapy unit 105.

In one aspect, pressure fluid used for positive pressure therapy may be heated by the thermal treatment element anywhere in the device. Therefore, the pressure fluid may provide both pressure therapy and thermal treatment.

The pressure fluid may be heated by the thermal treatment element before and/or during the therapy. Similarly, the pressure fluid may be heated by the thermal treatment element before the pressure fluid flows into the therapy unit 105 and/or after the pressure fluid flows into the therapy unit 105.

The pressure fluid may be heated up to a temperature in a range from 38° C. to 200° C., or from 38° C. to 150° C., or from 38° C. to 100° C., or from 38° C. to 80° C., or from 38° C. to 60° C., or from 38° C. to 50° C., or from 38° C. to 45° C., or from 45° C. to 100° C., or from 50° C. to 100° C. Therefore the temperature of the epidermis, dermis, hypodermis, muscle, adipose tissue and/or other tissues may rise and create a temperature gradient e.g. a positive temperature gradient, with the highest temperature on the epidermis.

The pressure fluid may be gas. The pressure fluid may be, for example, air, oxygen, or CO₂. The pressure fluid may be used both to introduce positive pressure changes in the therapy unit 105 and also to provide the thermal treatment.

The device may include pressure fluid, e.g. gas, liquid, a mixture of at least two types of gas, a mixture of at least two types of liquid and/or a mixture of at least one gas and one liquid. The gas may be a single type of gas, e.g. argon, helium, nitrogen, oxygen and/or moist oxygen. The gas may also be a mixture of different gases, e.g. air, or a mixture of argon and helium, argon and hydrogen, argon and oxygen, oxygen and hydrogen, nitrogen and oxygen, argon and oxygen, oxygen and ozone or argon and oxygen together with nitric oxide.

In one aspect, the thermal treatment may be provided by heating the pressure fluid by the thermal treatment element, wherein the pressure fluid may be located in the therapy unit 105. In one example, the pressure fluid may be heated by the thermal treatment element located inside, on, around or in the vicinity of the therapy chamber and configured to heat the pressure fluid inside the therapy chamber.

The thermal treatment element may be configured to apply heat to the tissue of the body part. The thermal treatment element may be configured to create a thermal gradient in the body part.

The thermal treatment may be provided by a flow of heated fluid (e.g. heated pressure fluid or heated different fluid than the pressure fluid) to the therapy unit 105. The heated fluid may have a flow in a range from 0.001 L/minute to 10,000 L/minute, or from 1 L/minute to 10,000 L/minute, or from 1 L/minute to 5000 L/minute, or from 50 L/minute to 1000 L/minute or from 100 L/minute to 1500 L/minute, or from 500 L/minute to 1500 L/minute, or from 800 L/minute to 1200 L/minute, or from 1000 L/minute to 1500 L/minute or from 1100 L/minute to 1300 L/minute.

In one aspect, the treatment element 108 may comprise a cold treatment element.

Cold treatment is widely used in the field of physiotherapy, as it is linked to various health benefits, such as speeding up the metabolism, reducing inflammation, swelling, and sore muscles, and/or improving immune response. It induces vasoconstriction, a process wherein blood vessels are narrowed, resulting from contraction of the muscular wall of the vessels. The blood flow is restricted, and the local blood perfusion decreases; thus, the body retains its body heat or increases its vascular resistance. It is primarily used to reduce pain after an injury, to speed the healing after an injury, to quicken recovery from exercise, to ease joint and muscle pain, and to reduce swelling. In another aspect, cooling of the body part may lead to freezing of adipose tissue and reduction of the fat in the body part (e.g. cryolipolysis).

The blood flow may be monitored directly or indirectly, invasively or non-invasively. For example by monitoring of a perfusion index, using oximetry, by monitoring blood oxygen level (SpO2), by a transmittance principle, by an optical principle, using a camera, or by any other appropriate blood flow monitoring method.

The subject's body part may be cooled to a temperature in a range from 10° C. to 35° C., or from 10° C. to 30° C., or from 10° C. to 25° C., or from 10° C. to 20° C., or from 10° C. to 15° C., or from 20° C. to 35° C., or from 25° C. to 35° C. or from 30° C. to 35° C., or from 32° C. to 35° C.

The temperature at the output of the cold treatment element may range from −40° C. to 35° C., or from −35° C. to 35° C., or from −35° C. to 20° C., or from −35° C. to 10° C., or from −35° C. to 0° C., or from −35° C. to −10° C., or from −35° C. to −20° C., or from −35° C. to −25° C., or from −30° C. to 0° C., or from −20° C. to 0° C., or from −10° C. to 0° C., or from −5° C. to 5° C., or from 0° C. to 10° C., or from 0° C. to 25° C., or from 0° C. to 35° C., or from 15° C. to 35° C.

The cold treatment may be provided in a continuous or alternating mode. The body part may be cooled to a preset temperature for a duration of time. The body part may be cooled for the whole duration of the vacuum compression therapy (pressure therapy). The temperature of the cooling may be constant during the time of vacuum compression therapy (pressure therapy) or the temperature may alternate. The body part may be cooled in a first period to a preset temperature, and then the cooling may stop in a second period. The period of cooling and not-cooling may alternate periodically.

The alternating cooling may have a profile that characterizes the temperature changes. The profile may be a square profile, a triangle profile, a ramp profile, a descending ramp profile, an exponential profile, an exponential decay profile and others. The profile may comprise a ramp-up time, a constant time and a ramp-down time.

The first period of cooling or the second period of non-cooling may have a duration in a range from 0.001 seconds to 10,000 seconds, or from 0.1 seconds to 5000 seconds, or from 1 second to 1000 seconds, or from 1 second to 500 seconds, or from 120 seconds to 1200 seconds, or from 120 seconds to 1000 seconds, or from 60 seconds to 600 seconds, or from 60 seconds to 360 seconds, or from 1 second to 120 seconds or from 1 second to 50 seconds or from 1 second to 20 seconds, or from 5 seconds to 15 seconds, or from 1 seconds to 10 seconds, or from 1 second to 5 seconds, or from 2 seconds to 3 seconds.

The cold treatment element may include an object filled with cooled fluid and/or a cooled fluid circulation unit, one or more thermoelectric coolers, one or more Peltier elements, one or more heat sinks, one or more heat conductors, and others. The cooled fluid may be a substance comprising air, CO₂, O₂, nitric oxide, nitrogen, water, distilled water, deionized water, water-doped NaCl, ethanol, oil, and others.

The cooled fluid may be a gas and may flow to the therapy unit 105.

The therapy unit 105 may comprise a heating device or a cooling device for direct heating or cooling the therapy unit 105 without preliminary heating or cooling the pressure fluid. The cooling device may be used when the therapy unit 105 is overheated.

In one aspect, pressure fluid used for positive or negative pressure therapy may be cooled by the cold treatment element anywhere in the device. Therefore, the pressure fluid may provide both pressure therapy and cold treatment.

The pressure fluid may be cooled by the cold treatment element before and/or during the therapy. Similarly, the pressure fluid may be cooled by the cold treatment element before the pressure fluid flows into the therapy unit 105 and/or after the pressure fluid flows into the therapy unit 105.

The pressure fluid may be cooled to a temperature in a range from −40° C. to 35° C., or from −35° C. to 35° C., or from −35° C. to 20° C., or from −35° C. to 10° C., or from −35° C. to 0° C., or from −35° C. to −10° C., or from −35° C. to −20° C., or from −35° C. to −25° C., or from −30° C. to 0° C., or from −20° C. to 0° C., or from −10° C. to 0° C., or from −5° C. to 5° C., or from 0° C. to 10° C., or from 0° C. to 25° C., or from 0° C. to 35° C., or from 15° C. to 35° C. Therefore the temperature of the epidermis, dermis, hypodermis, muscle, adipose tissue and/or other tissues may decrease and create a temperature gradient e.g. a negative temperature gradient, with the lowest temperature on the epidermis.

The pressure fluid may be, for example, air, oxygen, or CO₂. The pressure fluid may be used both to introduce positive pressure changes in the therapy unit 105 and also to provide the cold treatment.

The device may include pressure fluid e.g. gas, liquid, a mixture of at least two types of gas, a mixture of at least two types of liquid and/or a mixture of at least one gas and one liquid. The gas may be a single type of gas e.g. argon, helium, nitrogen, oxygen and/or moist oxygen. The gas may also be a mixture of different gases e.g. air, or a mixture of argon and helium, argon and hydrogen, argon and oxygen, oxygen and hydrogen, nitrogen and oxygen, argon and oxygen, oxygen and ozone or argon and oxygen together with nitric oxide.

In one aspect, the cold treatment might be provided by cooling the pressure fluid by the cold treatment element that is located in the therapy unit 105. The cold treatment may be provided.

The cooling unit may be configured to remove heat from the tissue and create a reverse thermal gradient.

The cold treatment may be provided by a flow of cooled fluid to the therapy unit 105. The cooled fluid may have a flow in a range from 0.001 L/minute to 10,000 L/minute, or from 1 L/minute to 10,000 L/minute, or from 1 L/minute to 5000 L/minute, or from 50 L/minute to 1000 L/minute or from 100 L/minute to 1500 L/minute, or from 500 L/minute to 1500 L/minute, or from 800 L/minute to 1200 L/minute, or from 1000 L/minute to 1500 L/minute or from 1100 L/minute to 1300 L/minute.

The system may comprise a refrigerant to cool the cooled fluid. The refrigerant may be any kind of refrigerant available, such as R-32, R-124a, R-410a, R-290, R-600a, R-717, R-1234yf, R-744, R-404a, R-134a, R-143a, R-407c, R-134a, R-410a, R-452a, R-404, R-404a, R-507, R-507a and others.

The system may comprise both thermal treatment and cold treatment. The thermal treatment element and/or cold treatment element may heat and/or cool the fluid that flows to the therapy unit 105. The thermal treatment element and/or cold treatment element may heat and/or cool the fluid in the therapy unit 105.

The cold treatment and/or thermal treatment element may comprise a nozzle. The nozzle may be configured to spray the cooled and/or heated fluid inside the therapy unit 105 and create a humid environment. The body part may be sprayed by the cooled and/or heated fluid from the nozzle. The cooled fluid and/or the heated fluid may be liquid such as water or distilled water.

In one aspect, a contrast treatment comprising a sequence of alternating heating and cooling periods may be provided to treat a target tissue of the subject. The contrast treatment may be provided by both a thermal treatment element and a cold treatment element. The alternating periods deliver and remove heat from the tissue, respectively. Contrast treatment is a well-established approach in physiotherapy used to treat pain and swelling and aid healing. It may be useful when the induction of vasoconstriction and/or vasodilatation mechanisms are beneficial. The induction of those processes may improve the overall blood circulation. Contrast treatment also affects the lymphatic system by dilating and constricting the lymph vessels, and improving the circulation of the lymph, resulting in quickening the healing of the injured or damaged tissue. Some of the common conditions treated by contrast treatment are ankle sprains, rheumatoid arthritis, carpal tunnel syndrome, soft tissue trauma, foot sprains, pain, and others.

The treatment element 108 may comprise other sources of treatment energy. The device and method may provide one or more treatment energies in order to provide treatment to the subject. The treatment energy may comprise microwave energy, radiofrequency energy (RF), ultrasonic energy, mechanical wave energy, thermal energy, light energy, plasma, magnetic field, and/or electric current. Different therapy energy may be used individually and/or may be combined. The device may comprise multiple additional treatment elements 108 that may provide various types of treatment energies in combination.

When the system comprises one or multiple additional treatment elements 108, the device may provide one or multiple types of treatment energies with the therapy unit 105 at the same time, sequentially or in overlay, for at least part of the duration of the vacuum compression therapy (pressure therapy) or for the whole duration of the vacuum compression therapy (pressure therapy). In one aspect, the device may provide vacuum compression therapy (pressure therapy) in combination with thermal treatment. In another aspect, the device may provide vacuum compression therapy (pressure therapy) in combination with cooling treatment.

In one aspect, the device may provide the thermal treatment before or after the vacuum compression therapy (pressure therapy).

In another aspect, the device may provide cooling treatment before or after vacuum compression therapy (pressure therapy).

In yet another aspect, the device may provide any treatment energy before or after vacuum compression therapy (pressure therapy).

The treatment element 108 may comprise a therapeutic fluid. The therapeutic fluid may be a therapeutic gas, such as oxygen or carbon dioxide. The therapeutic fluid (e.g. therapeutic gas) may flow to the therapy unit 105 by at least one pump or a nozzle. The therapeutic fluid (e.g. therapeutic gas) may be used to hasten the healing of lesions, wounds, or ulcers on the subject's body part. The therapeutic fluid (e.g. therapeutic gas) may also be used to treat various medical, physiological, or aesthetic conditions.

The treatment element 108 may be used to treat the body part by an electromagnetic field. The electromagnetic field may provide a treatment energy to the body part.

Energy flux of the electromagnetic field may be in the range of 0.001 W·cm⁻² to 1500 W·cm⁻², or in the range of 0.01 W·cm⁻² to 1000 W·cm⁻², or in the range of 0.5 W·cm⁻² to 500 W·cm⁻².

Application of electromagnetic energy may heat the tissue in the range of 37.5° C. to 80° C. or 37.5° C. to 70° C. or 37.5° C. to 60° C. or 37.5° C. to 55° C. or 38° C. to 50° C. or 39° C. to 45° C.

The treatment element 108 may comprise a radiofrequency radiating element (e.g. a transducer, a coil or an electrode). An electromagnetic field used for treating the tissue may be a radiofrequency field, typically in a range from 10 kHz to 300 GHz, or 100 kHz to 3 GHz, or 500 kHz to 3 GHz, 400 kHz to 900 MHz or 500 kHz to 900 MHz or 500 kHz to 100 MHz or 500 kHz to 50 MHz or 650 kHz to 30 MHz or 720 kHz to 8 MHz or around 13.56 MHz, 40.68 MHz, 27.12 MHz, or 2.45 GHz. The wavelength λ of the radiofrequency field may be in a range from 1 mm to 1000 km, or from 1 mm to 100 km, or from 1 mm to 30 km, or from 100 mm to 3 km, or from 100 mm to 600 m, or from to 600 m, or from 333 mm to 750 m, or from 333 mm to 600 m, or from 3 m to 600 m, or from 6 m to 600 m, or from 10 m to 461 m, or from 37.5 m to 416 m, or from 1 mm to 10 km, or from 10 mm to 1 km, or from 100 mm to 100 m, or from 100 m to 1 km, or from 1 mm to 100 mm.

The output power of the radiofrequency field may be in a range of 0.1 W to 1000 W, or 1 W to 400 W or 2 W to 370 W, or 5 W to 300 W, or 5 W to 250 W, or 8 W to 180 W or 10 W to 150 W.

The treatment element 108 may comprise at least one microwave radiating element. An electromagnetic field used for treating the tissue may be a microwave field, typically in a range from 300 MHz to 300 GHz, 400 MHz to 150 GHz, or 500 MHz to 100 GHz, 700 MHz to 70 GHz, 1 GHz to 30 GHz, 1 GHz to 10 GHz.

The wavelength λ of the microwave field may range from 1 mm to 1 m, or 2 mm to 75 cm, or 3 mm to 60 cm, or 4.3 mm to 43 cm, or 1 cm to 30 cm, or 3 cm to 30 cm.

The electromagnetic radiation may have a frequency within various ISM bands, such as, for example, 6.78 MHz, 13.56 MHz, 26.957 MHz, 40.66 MHz, 433.05 MHz, 902 MHz, 2.4 GHz, 5.725 GHZ, 24 GHZ, 61 GHz, 122 GHz or 244 GHz.

The treatment element 108 may comprise a light radiating element (e.g. light emitting diode, laser, flashlamp, or a light bulb). An electromagnetic field used for treating the tissue may be light.

The light may have a frequency in a range from 1 THz to 1500 THz, or from 10 THz to 1000 THz, or from 100 THz to 1000 THz, or from 400 THz to 790 THz.

The wavelength λ of the light may range from 100 nm to 15,000 nm, or from 200 nm to 12,000 nm, or from 300 nm to 11,000 nm, or from 400 nm to 10,600 nm, or from 380 nm to 750 nm, or from 200 nm to 1140 nm, or from 290 nm to 980 nm, or from 320 nm to 850 nm, or from 400 nm to 700 nm, or from 1000 nm to 11,000 nm, or from 1064 nm to 10,650 nm or from 1900 nm to 5800 nm, or from 2080 nm to 4100 nm.

The light treatment involves irradiating a target area to be treated with radiation at one or more light wavelengths preferentially absorbed by the chromophores in a target area.

The light treatment may provide heating, myorelaxation effects and/or biostimulation. Light in the range of 620 to 750 nm may be beneficial for local circulation enhancement or restoration of connective tissue. Light in the range of 400 to 500 nm may provide a bactericidal effect. Light in the range of 560 to 600 nm may stimulate tissue rejuvenation. All these types of light may be applied.

Light may be monochromatic or polychromatic. Light may be applied in pulses with a duration in the range of 0.1 μs to 10000 ms, or in the range of 1 μs to 5000 ms, or in the range of 2 μs to 2500 ms, or in the range of 5 μs to 1000 ms.

The energy flux provided by the light may be in the range of 0.005 W·cm⁻² to 75 W·cm⁻², or in the range of 0.01 W·cm⁻² to 60 W·cm⁻² or in the range of 0.01 W·cm⁻² to 50 W·cm⁻². Methods and devices may also include a spot size defined as an area of the surface of tissue treated by the light. The spot size may be in the range of 0.01 cm² to 1000 cm², or in the range of 0.05 cm² to 800 cm², or in the range of 0.1 cm² to 750 cm².

According to one aspect of the device, a low-level light may be used. The output of the light source may be in the range of 0.1 mW to 600 mW, or in the range of 1 mW to 500 mW, or in the range of 1.5 mW to 475 mW, or in the range of 3 mW to 450 mW. The energy flux provided by the low-level light may be in the range of 0.01 W·cm⁻² to 30 W·cm⁻², more preferably in the range of 0.05 W·cm⁻² to 25 W·cm⁻² and most preferably in the range of 0.1 W·cm⁻² to 20 W·cm⁻².

According to another embodiment, a high-level light may be used. In this case, the output of the light source may be in the range of 0.1 W to 30 W, or in the range of 0.2 W to 25 W, or in the range of 0.35 W to 15 W. The energy flux provided by the high-level light may be in the range of 0.01 W·cm⁻² to 50 W·cm⁻², or in the range of 0.05 W·cm⁻² to 40 W·cm⁻² or in the range of 0.1 W·cm⁻² to 35 W·cm⁻².

Electromagnetic energy may also be applied in a narrow spectral band. Some of the spectral bands may represent different colors of the visible part of the electromagnetic spectrum. The wavelength of the applied light may be close to 254 nm, 405 nm, 450 nm, 530 nm, 560 nm, 575 nm, 640 nm, 685 nm, 830 nm and/or 1064 nm. The term “close to” refers to a deviation of 20% or less, more preferably 15% or less, and most preferably 10% or less from the nominal wavelength.

The treatment by electromagnetic field may be used to heat the soft tissue of the body part of the subject. The treatments may be used for enhancing the visual appearance of the skin and body by, for example, increasing production of collagen and/or elastin, smoothing the skin, reducing cellulite, and/or removing adipose cells. The treatment by electromagnetic field may aid in decreasing joint stiffness, reducing pain, relieving muscle spasms, reducing inflammation, reducing edema and increasing blood flow to the body part.

In one aspect, the body part may be treated by at least one treatment element 108. In another aspect, the body part may be treated by at least two treatment elements 108, forming an array of the treatment elements 108. The at least one treatment element 108 may be in direct contact with the surface of the skin or may be spaced from the subject's body, e.g. by an air gap, spacing element or spacing material, cooling element or cooling material. At least one treatment element 108 may radiate electromagnetic energy with different frequencies at the same time or at least one treatment element 108 may be alternatively switched on/off during the treatment.

Heating by the treatment element 108 may be combined with cooling of the surface of the skin of the body part by another treatment element 108 and thus creating a thermal reverse gradient in the body part. This can be done using external mechanisms to remove heat from the tissue close to the skin's surface. For example, various cooling mechanisms, such as water bolus, can be used to cool the skin surface and prevent damage or heat-induced injuries.

The temperature of the body part may be monitored using one or more temperature sensors. The temperature sensor can be a thermometer, including a resistance thermometer or an infrared thermometer, a thermocouple, a thermistor, or an optical sensor. One or more temperature sensors may be used to measure the temperature of the body part, the skin surface, the therapy unit 105 and/or the treatment elements 108.

The temperature may be evaluated indirectly. Indirect measurement of temperature may be a measure of a different physical parameter other than temperature. Such parameters can, for example, be the impedance of the tissue and/or others.

The treatment element 108 may comprise a magnetic field generating device, such as, for example, a magnetic coil. Muscle stimulation by a time-varying magnetic field provides several benefits over known methods for treating biological structures, and allows for non-invasive stimulation of muscles located beneath other muscles. Further, time-varying magnetic fields may be used to provide muscle stimulation to cause muscle contraction through a thick layer of adipose tissue.

The magnetic field may be a time-varying magnetic field such that a muscle in the body area of the subject is contracted. The time-varying magnetic field may have a magnetic flux density in a range of 0.1 Tesla to 7 Tesla, or from 0.1 Tesla to 5 Tesla, or from 0.2 Tesla to 4 Tesla, or from 0.3 Tesla to 3 Tesla, or from 1 Tesla to 3 Tesla.

A repetition rate may refer to the frequency of firing the magnetic impulses. The repetition rate may be derived from the time duration of the magnetic pulse The time-varying magnetic field may have a repetition rate in a range from 0.1 Hz to 700 Hz, or from 1 Hz to 700 Hz, or from 1 Hz to 500 Hz, or in the range of 1 Hz to 300 Hz, or 1 Hz to 150 Hz.

The duration of magnetic impulses may be in a range from 1 μs to 250 s, or from 1 μs to 100 s, or from 1 μs to 10 s, or from 1 μs to 1 s, or from 1 μs to 10 ms, or from 3 μs to 3 ms, or from 3 μs to 3 ms, or from 3 μs to 1 ms, or 10 μs to 2000 μs, or 50 μs to 1000 μs, or from 100 μs to 800 μs.

The magnetic field may be applied in trains and bursts. The treatment duty cycle may be higher than 10%, which means the treatment regime is interrupted with a ratio of up to 1 active to 9 passive time intervals. The treatment duty cycle of a magnetic field may be at least 10, 15, 20, 25, 40, 50, 75, 85, or 90%. The magnetic impulse duration may be in the range of 3 μs to 10 ms or more, or alternatively 3 μs to 3 ms or alternatively 3 μs to 1 ms. The impulse duration may be, for example, 3, 10, 50, 200, 300, 400, 500, 625, 1000, 2000 or below 3000 μs.

In one aspect, the treatment element 108 may comprise a mechanical treatment element. The mechanical treatment unit may provide mechanical energy. Mechanical energy may provide focused and/or unfocused heating, cavitation, microbubble formation, muscle stimulation, stimulation of the healing process, blood flow stimulation and/or stimulation of an inflammatory response.

Optionally, the application of the mechanical energy may lead to the creation of focus in a desired depth of the tissue and/or defocusing the energy into a larger volume of the tissue. The treatment depth of the mechanical energy may be in the range of 0.1 mm to 100 mm, or in the range of 0.2 mm to 50 mm, most preferably in the range of 0.25 mm to 25 mm, or in the range of 0.3 to 15 mm under the surface of the treated tissue. Treatment depth may be influenced by focusation, defocusation and/or frequency of mechanical energy.

According to one aspect of the invention, the treatment element 108 may be an ultrasound radiating element providing ultrasound energy. The frequency of ultrasound energy may be in the range of 20 kHz to 25 GHz, or in the range of 20 kHz to 1 GHz, or in the range from 50 kHz to 250 MHz, or in the range of 100 kHz to 100 MHz. Energy flux provided by ultrasound energy may be in the range of 0.001 W·cm⁻² to 500 W·cm⁻², or in the range of 0.005 W·cm⁻² to 350 W·cm⁻², or in the range of 0.05 W·cm⁻² to 250 W·cm⁻².

Mechanical energy may also be shock wave energy, where shock waves may provide pain relief, blood flow enhancement, myorelaxation and mechanical stimulation.

Shock waves are characterized by step pressure amplitude growth in comparison to the surrounding pressure and non-linear wave propagation. Also, shock waves are characterized by a swift positive pressure increase with positive peak pressure amplitudes in the range of 0.1 MPa to 150 MPa or 3 MPa to 150 MPa or 7 MPa to 150 MPa. Shock wave energy may be generated by electrohydraulic, piezoelectric, electromagnetic, pneumatic and/or ballistic principles. The repetition rate of shock wave energy may be in the range of 0.1 Hz to 1000 Hz, or in the range of 0.1 Hz to 750 Hz, or in the range of 0.5 Hz to 600 Hz or in the range of 1 Hz to 500 Hz. Energy flux provided by shock wave energy may be in the range between 0.0001 W·cm⁻² and 50 W·cm⁻², or in the range between 0.0001 W·cm⁻² and 35 W·cm⁻², or in the range between 0.0001 W·cm⁻² and 25 W·cm⁻².

In one aspect, the treatment element 108 may comprise an electric energy treatment element. The electric energy element may provide electric energy, which may provide muscle stimulation, analgesic effects, contraction of muscles and/or myorelaxation effects.

Electric energy may be applied in a constant current mode. In this case, the output current may be in the range of 0.01 mA to 400 mA or 0.01 mA to 300 mA, or 0.01 mA to 180 mA. Output voltage may be in the range of 0.001 V to 350 V or 0.001 V to 300 V or 0.001 V to 250 V.

Electric energy may be applied in a constant voltage mode. In this case, the output current may be in the range of 0.01 mA to 400 mA or 0.01 mA to 300 mA or 0.01 mA to 180 mA. Output voltage may be in the range of 0.001 V to 350 V or 0.001 V to 300 V or 0.001 V to 250 V.

Electric energy may provide a high voltage therapy. In this case, the output current may be in the range of 0.01 mA to 50 mA or 0.01 mA to 30 mA or 0.01 mA to 15 mA. Output voltage may be in the range of 0.001 V to 800 V or 0.001 V to 650 V or 0.001 V to 600 V.

In a microcurrent application, the output current may be in the range of 0.001 mA to 10 mA or 0.001 mA to 5 mA or 0.001 mA to 3 mA. Output voltage may be in the range of 0.001 V to 250 V or 0.001 V to 200 V or 0.001 V to 150 V.

Electric energy (e.g. electric current) may be applied in pulses.

The repetition rate of pulses used for treatment by the electric current may have a repetition rate in a range of 0.1 Hz to 12 kHz, 1 Hz to 6 kHz, 10 Hz to 1 kHz, 25 Hz to 500 Hz, 60 Hz to 350 Hz, or 75 Hz to 280 Hz.

The treatment by the electric current may be used for muscle stimulation, inducing pain relief, myorelaxation, or others.

Methods of treatment may further include an application of at least one substance, wherein the substance may affect the tissue. The substance may be applied before, during and/or after the application of a pressure gradient and/or an additional type of energy. The substance may be applied as aerosol contacting the body, orally, and/or intradermally. The substance may improve treatment results, make treatment more comfortable by, for example, minimizing pain during the treatment, and/or make treatment safer. The substance may include oleoresin capsicum (pelargonic acid vanillylamide), increasing blood flow in the skin. Other substances may include lipolysis augmentation agents like methyl xanthines, such as theophylline, aminophylline, caffeine; pentoxifylline; beta-1 adrenergic agonists (stimulators) including, but not limited to: forskolin, norepinephrine, epinephrine, isoproterenol; specific beta-3 agonists (stimulators) including but not limited to: fenoterol, clenbuterol; alpha-2 adrenergic inhibitors (antagonists) including but not limited to: yohimbine, rauwolscine, oxymetazoline, piperoxane, phentolamine, dihydroergotamine, idazoxin; adenosine inhibitors; calmodulin agonists; thyroid hormones including but not limited to: T3/triiodothyronine and T4/tetraiodothyronine; sex hormones including but not limited to methyltestosterone; prostaglandin inhibitors including but not limited to aspirin (ASA), non-steroidal anti-inflammatory drugs (NSAID's), and finasteride, tamoxifen, catecholamine, flavonols and the like. The named substances are not a complete list of possible substances, and other substances may be used to provide some or all of the effects described above.

Substances may include alkaloids (e.g. xanthines), antithyroid agents, metformin and/or octreotide modulating normal metabolism and/or basal metabolism rate of the subject's body by acceleration of the metabolism related to the apoptosis. Substances may include prostaglandins and their analogs, modified lipids (e.g. lysophosphatidylserine, lipoxins, resolvins, protectins and/or maresins), lipoprotein lipase inhibitors, nitric oxide secretion stimulators, antioxidants (e.g. ascorbic acid), or derivatives of carbohydrates providing improvement of removal of dying cells.

Substances may include terpenes (e.g. forskolin), catecholamines, hormones (e.g. leptin, growth hormone and/or testosterone), alkaloids (e.g. synephrin), phosphodiesterase inhibitors (e.g. xanthins), polyphenols, peptides (e.g. natriuretic peptides), amino acids providing improvement of the lipolysis rate. Substances may also include anesthetics e.g. lidocaine, benzocaine, and menthol, providing reversible decrease or absence of sensation in the specific part of the subject's body.

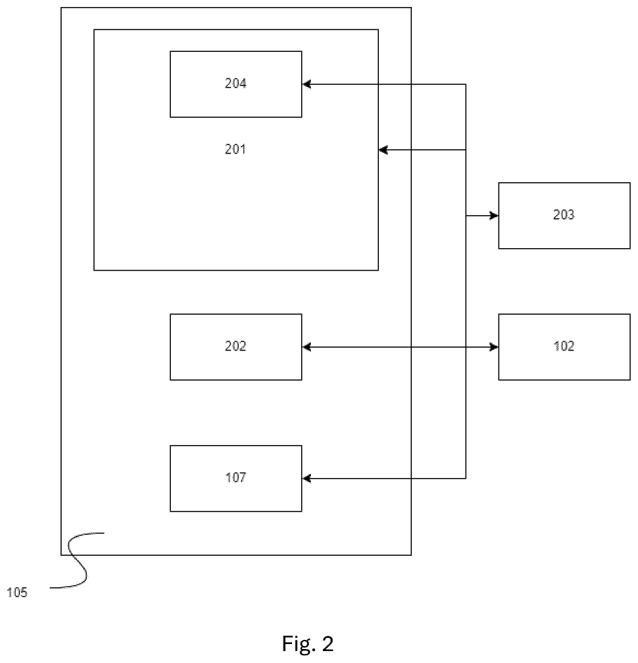

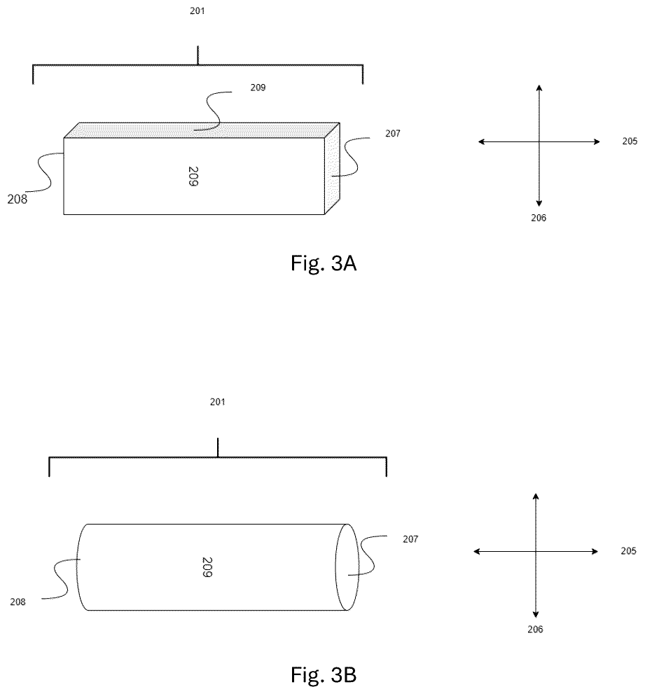

FIG. 2 shows one exemplary schematic diagram of the therapy unit 105. The therapy unit 105 may comprise a therapy chamber 201, a sealing unit 202, at least one sensor 107, and at least one opening 204. The therapy unit 105 may be in communication with the power supply 102 and at least one fluid conduit 203 (e.g. pipe, tube, hose). The therapy chamber 201 may have various shapes and sizes. The therapy chamber 201 may be in the form of a cylinder or a prism, or have an oval shape, an hourglass shape, or a tubular shape which is narrower in the middle and wider at its ends. The therapy chamber 201 may have at least one opening 204.

The fluid conduit 203 may be rigid or flexible.

In another aspect, the fluid conduit 203 may comprise flexible parts and rigid parts, e.g. a flexible tube with rigid connectors at the ends configured to be connected to pressure generator 106 and therapy chamber 201 or therapy unit 105.

The therapy chamber 201 may comprise a rigid material like metal, plastic, glass, poly(methyl methacrylate), PMMA, other polymers and/or other rigid materials. It may also be from a flexible material and/or an inflatable material. The therapy chamber 201 may be foldable, providing easy storage options.

The therapy chamber 201 may be at least partially transparent. The therapy chamber 201 may be made out of an opaque material.

In one aspect, the therapy chamber 201 may be made of at least one part. In another aspect, the therapy chamber 201 may be made of at least two parts and/or at least three parts. The device may comprise one or more therapy chambers 201. The therapy chamber 201 may be used to provide therapies to various body parts at the same time.

The therapy chamber 201 may be configured to enclose a part of subjects' body. The therapy chamber 201 may be coupled to the sealing unit 202. The therapy chamber 201 may be configured to seal a part of subjects' body.

In one aspect the device may comprise multiple therapy chambers 201.

The multiple therapy chambers 201 may be used to treat various body parts. The multiple therapy chambers 201 may be each configured to treat a specific body part, for example arm, hand, leg, feet, trunk or thigh, abdomen, genital tissue, head, neck, lower part of the body, upper part of the body or others. The therapy in one of the multiple therapy chambers 201 may begin first, and the therapy in the second one of the multiple therapy chambers may begin second. The multiple therapy chambers 201 may start the therapy at the same time. The multiple therapy chambers 201 may be used to treat various body parts simultaneously.

The multiple therapy chambers 201 may provide different therapies to the body part.

In one aspect of the invention, all of the multiple therapy chambers 201 may be configured to provide positive and negative pressures.

In another aspect of the invention, at least one of the therapy chambers 201 may be configured to provide additional treatment by the treatment element 108.

In another aspect, the multiple therapy chambers 201 may provide different treatment patterns of the additional treatment.

Yet in another aspect, the multiple therapy chambers 201, may provide the same treatment patterns of the additional treatment.

In another aspect of the invention, the device may comprise a first therapy chamber 201 and a second therapy chamber 201. The first therapy chamber 201 may be used to provide negative pressure to the body part. The second therapy chamber 201 may be used to provide positive pressure to the body part.

In another aspect of the invention, the device may comprise a first therapy chamber 201 and a second therapy chamber 201. The first therapy chamber 201 may be configured to provide additional treatment by the treatment element 108 and the second therapy chamber 201 may be configured to provide positive pressure, or negative pressure, or positive and negative pressure to the body part.

The sealing unit 202 may be configured to seal the body part in the therapy chamber 201 and ensure airtight, safe, and comfortable sealing of the body part without any pressure leaks from or to the therapy chamber 201.

The therapy chamber 201 may have a length in a range from 1 cm to 250 cm, or from 25 cm to 150 cm, or from 50 cm to 120 cm, or from 60 to 100 cm, or from 70 to 90 cm.

The therapy chamber 201 may have a width in a range from 1 cm to 200 cm, or from 1 cm to 100 cm, or from 15 cm to 75 cm, or from 25 cm to 50 cm, or from 25 to 30 cm.

The diameter of the therapy chamber 201 may be in a range from 1 cm to 100 cm, or from 15 cm to 75 cm, or from 25 cm to 50 cm, or from 25 cm to 40 cm, or from 25 cm to 35 cm, or from 25 cm to 30 cm.

The device may comprise at least one wrap for sealing the space around the patient's body part, that is inserted into the therapy chamber 201. In one aspect, the sealing unit 202 may comprise the at least one wrap. In another aspect, the at least one wrap may be applied on the patient's body part (or at least on some portion of the body part) externally prior to the insertion of the body part into the sealing unit 202, for example the wrap may be applied on the portion of the body part which is in contact with the sealing unit 202 during the treatment. The wrap may be configured to spread the pressure of the sealing mechanism and thus reduce skin damage on the patient's body or to provide cover for the sealing unit, e.g. to enhance the sanitation of the sealing unit. For example, the wrap may be configured as a disposable or single-use component, thereby eliminating the need to clean the sealing unit after each therapy session, as the wrap itself serves as a sanitary barrier.

The wrap may be made of rubber, neoprene, latex, synthetic rubber, e.g. NBR rubber, silicone, or any other appropriate material.

The wrap may have a form of a strip or tape. The wrap may have a shape of rectangle, square, or any other appropriate shape. The wrap may have sharp corners, or may be rounded.

The length of the wrap may be in the range of 2 cm to 200 cm, or in the range of 5 cm to 180 cm, or in the range of 8 cm to 150 cm, or in the range of 10 cm to 100 cm.

The width of the wrap may be in the range of 1 cm to 30 cm, or in the range of 2 cm to 25 cm, or in the range of 3 cm to 20 cm, or in the range of 4 cm to 18 cm, or in the range of 5 to 15 cm.

The thickness of the wrap may be in the range of 0.1 mm to 30 mm, or in the range of 0.5 mm to 25 mm, or in the range of 0.8 mm to 22 mm, or in the range of 1 mm to 20 mm.

In one aspect, the wrap may comprise a fastening member configured to fasten the wrap around the body part (or at least a portion of the body part). The fastening member may be Velcro (hook and loop fasteners), snaps, buckles, zippers, hooks and eyes, magnets, elastic bands or straps, adhesive strips, silicone grip strips, buttons and loops, drawstrings or laces, twist-lock or cam-lock closures, ratchet straps, Boa® closure systems, or reusable hook-and-grip systems, depending on the desired level of adjustability, reusability, and ease of use.

One type of wrap may be used in combination with various types of sealing mechanism, for example for the cuff, the moldable membrane, the flexible membrane, or the folded cuff.

The different therapy chambers 201 may be used for the therapy of various body parts or therapy purposes.

The therapy chamber 201 may be removably connected to the therapy device. The at least two therapy chambers 201 may be interchangeable according to the needs of the therapy, or the body parts of the subject of the therapy, or based on parameters of the therapy chambers 201 including for example length, diameter, or any other appropriate parameter.

In one aspect, the therapy chamber 201 for therapy of the torso may be up to about 200 cm long and up to about 150 cm wide.

In another aspect, a therapy chamber 201 for therapy of the abdomen and/or chest may be up to about 100 cm long and up to about 150 cm wide.

In still another aspect, a therapy chamber 201 for therapy of the neck may be up to about 50 cm long and up to about 40 cm wide.

In still another aspect, a therapy chamber 201 for therapy of one leg may be up to about 90 cm long and up to about 40 cm wide.

In still another aspect, a therapy chamber 201 for therapy of one leg may be up to about 80 cm long and up to about 29 cm wide.

In still another aspect, a therapy chamber 201 for therapy of one leg may be up to about 100 cm long and up to about 50 cm wide.

In still another aspect, a therapy chamber 201 for therapy of one leg may be up to about 150 cm long and up to about 50 cm wide.

In still another aspect, a therapy chamber 201 for therapy of both legs may be up to about 150 cm long and up to about 100 cm wide.

In still another aspect, a therapy chamber 201 for therapy of both thighs may be up to about 50 cm long and up to about 100 cm wide.

In still another aspect, a therapy chamber 201 for therapy of one thigh may be up to about 50 cm long and up to about 50 cm wide.

In one aspect the therapy chamber 201 may have a cylindrical cross-section. The therapy chamber 201 may comprise an outer diameter and an inner diameter. The inner diameter of the chamber may be up to 150 cm, or up to 100 cm, or up to 50 cm, or up to 30 cm.

In some aspects, the inner diameter may be up to 99 or 97 or 95 or 90% of the outer diameter of the cylinder.

One or more sensors 107 may be positioned in the therapy chamber 201, in the sealing unit 202 or on the subject's anatomical body part to be treated, and at any other positions on the device and/or subject.

The therapy unit 105 may automatically reach the optimal position for the body part of the patient, for example the horizontal position, wherein at least one sensor may indicate the body part position. The therapy unit 105 may be moved to the requested position based on information from the at least one sensor.

In another aspect, the therapy unit 105 may automatically reach the optimal position for the body part of the patient inside the sealing unit 202 to provide the best sealing or the most comfortable treatment.

In one aspect, the device may comprise distance sensors, e.g. light (optical, infrared, laser, time-of-flight (ToF)) sensors, ultrasound sensors, capacitive sensors, or electric field sensors. In one aspect the distance sensors may be in the range of 2 to 10, or in the range of 2 to 7 or in the range of 3 to 5, or 3, or 4, or 5, and may be positioned around or adjacent the entrance of the sealing unit at the same or different distance. When the body part is inserted into the sealing unit the distance sensors may measure a distance of the body part form each distance sensor and the control unit may determine this information and adjust the position of the therapy unit such, that the distance of the body part from each distance sensor is the same or almost the same or in a proper preselected range. In such case the body part should be right in the middle of the sealing unit, which could provide the best sealing or the highest comfort for the patient.

In another aspect, the sealing unit may comprise a number of pressure sensors in the range of 2 to 10, or in the range of 2 to 7 or in the range of 3 to 5, or 3, or 4, or 5, which may be in contact with the body part when inserted into the sealing unit. The control unit may adjust the position of the therapy unit such that the pressure on each pressure sensor is the same or almost the same. In such case the body part should be right in the middle of the sealing unit, which would provide the best sealing or the highest comfort for the patient.

In another aspect, the sealing unit may comprise a moldable membrane and actuators which may be in contact with the body part that is inserted into the moldable membrane. The control unit may determine a position of at least some actuators when they are in the sealing position. The position can be determined based on a number of actuators that may be in the range of 2 to 10, or in the range of 2 to 7 or in the range of 3 to 5, or 3, or 4, or 5. In one aspect the control unit may determine a linear position of the actuators, e.g. for linear actuators. In another aspect the control unit may determine a rotation of actuators, e.g. for rotary actuators. Yet in another aspect, the control unit may determine an angle of a lever for levered actuators. By determining the position of the measured actuators, the control unit may adjust the position of the therapy unit such that the body part is right in the middle of the sealing unit, which would provide the best sealing or the highest comfort for the patient.

In another aspect, the sealing unit (e.g. actuators) may comprise strain gauges and the pressure on strain gauges would vary according to the deflection of the body part inside the sealing unit. The number of the strain gauges may be in the range of 2 to 20, or in the range of 2 to 15 or in the range of 3 to 5, or 3, or 4, or 5. The control unit would then determine the values of each strain gauges and adjust the position of the therapy unit such that the pressure on the strain gauges would correspond to the optimal position of the body part inside the sealing unit, e.g. the pressure on the strain gauges is same or almost the same.

The therapy unit 105 position may be adjusted by pressing a button and/or by touching the user interface, for example the control panel with display, by a person providing the therapy.

The at least one sensor may indicate the correct or incorrect position of the body part of the patient in the sealing unit 202 and/or may signal the user to change the position of the therapy unit 105 relative to the patient's body part. A subject support may be located inside of the therapy chamber 201, or outside the therapy chamber 201. The subject support may be configured to comfortably support the subject and/or the body part inside the therapy chamber 201. The subject support may be a cushion, pillow, pad, mat and/or underlay.

The device according to the invention may comprise a plurality of at least two subject supports located inside the therapy chamber 201 and/or outside the therapy chamber 201.

The device may include an element providing and/or eliminating water vapor from the therapy chamber 201. Water vapor may be provided and/or eliminated according to data from the humidity sensor.

The device according to the invention may comprise a valve.

The valve may be manual, pneumatic, hydraulic, electromagnetic, or electromechanical or any other appropriate valve. The valve may be a solenoid valve.

The main unit 101 may comprise at least one valve. The at least one valve may be in communication with the pressure generator 106 and with the control unit 103. The therapy chamber 201 may comprise at least one valve.

In one aspect, the therapy chamber 201 may comprise a valve that provides both an input and an output. The valve may be in communication with the pressure generator 106. The valve may be in communication with the control unit 103.

In another aspect, the therapy chamber 201 may comprise two valves, the first valve providing both an input and an output. The second valve may be in communication with at least one sensor 107. The at least one sensor 107 may be for example a pressure sensor or a flow sensor. The two valves may be in communication with the control unit 103.

In another aspect, the therapy chamber may comprise two valves, the first valve providing an input and the second valve providing an output. The two valves may be in communication with the pressure generator 106. The two valves may be in communication with the control unit 103.

In another aspect, the therapy chamber may comprise three valves, the first valve providing an input and the second valve providing an output. The third valve may be in communication with at least one sensor 107. The at least one sensor 107 may be for example a pressure sensor or a flow sensor. The three valves may be in communication with the control unit 103.

The pressure generator 106 may be able to introduce periods of various pressure in the therapy chamber 201. The therapy chamber may comprise periods of various pressures. Pressure generator 106 may provide an alternating pressure, such that a first period of non-atmospheric pressure is followed by a second period of non-atmospheric pressure.

Pressure generator 106 may be, for example, a pump, a fan, a compressor, or a blower. A pressure generator 106 may be used to produce positive pressure, or negative pressure, or both positive pressure and negative pressure.

In one aspect, the device may comprise multiple pressure generators 106 used for the same pressure generation.

In another aspect, the device may comprise different pressure generators 106, e.g. one for positive pressure and second one for negative pressure generation.

The pressure generator 106 may be connected to the therapy unit 105 by at least one fluid conduit (e.g. pipe, tube, or hose) or a system of fluid conduits.

The pressure generator 106 may be able to provide periods of various pressure in the therapy unit 105. The therapy unit 105 may comprise periods of various pressures.

Pressure generator 106 may provide an alternating pressure, such that a first period of non-atmospheric pressure is followed by a second period of non-atmospheric pressure. The first period of non-atmospheric pressure may be a period with a pressure higher than atmospheric pressure. The second period of the non-atmospheric pressure may be a period with pressure lower than atmospheric pressure.

The first period of non-atmospheric pressure may have a duration in a range from 0.001 seconds to 10,000 seconds, or from 0.1 seconds to 5000 seconds, or from 1 second to 1000 seconds, or from 1 second to 500 seconds or from 1 second to 100 seconds or from 1 second to 50 seconds or from 1 second to 20 seconds, or from 5 seconds to 15 seconds, or from 1 seconds to 10 seconds, or from 1 second to 5 seconds, or from 2 seconds to 4 seconds, or from 5 to 10 seconds, or from 5 to 25 seconds, or from 3 to 7 seconds, or from 8 to 14 seconds, or from 10 to 60 seconds, or from 10 to 40 seconds, or from 10 to 30 seconds, or from 10 to 25 seconds, or from 10 to 20 seconds, or from 10 to 16 seconds.

The second period of non-atmospheric pressure may have a duration in a range from 0.001 seconds to 10,000 seconds, or from 0.1 seconds to 5000 seconds, or from 1 second to 1000 seconds, or from 1 second to 500 seconds or from 1 second to 100 seconds or from 1 second to 50 seconds or from 1 second to 20 seconds, or from 5 seconds to 15 seconds, or from 1 seconds to 10 seconds, or from 1 second to 5 seconds, or from 2 seconds to 4 seconds, or from 5 to 10 seconds, or from 5 to 25 seconds, or from 3 to 7 seconds, or from 8 to 14 seconds, or from 10 to 60 seconds, or from 10 to 40 seconds, or from 10 to 30 seconds, or from 10 to 25 seconds, or from 10 to 20 seconds, or from 10 to 16 seconds.

The pressure difference between the pressure in the therapy chamber of the therapy unit 105 and the atmospheric pressure in the first period of non-atmospheric pressure may be in a range from 0.001 kPa to 1000 kPa, from 0.01 kPa to 1000 kPa, or from 0.01 kPa to 100 Pa, or from 0.1 kPa to 100 kPa, or from 1 kPa to 100 kPa, or from 1 kPa to 20 kPa, or from 10 kPa to 50 kPa, or from 5 kPa to 20 kPa, or from 10 kPa to 15 kPa, or from 20 kPa to 30 kPa.

The pressure difference between the pressure in the therapy chamber of the therapy unit 105 and the atmospheric pressure in the second period of non-atmospheric pressure may be in a range from 0.001 kPa to 1000 kPa, from 0.01 kPa to 1000 kPa, or from 0.01 kPa to 100 Pa, or from 0.1 kPa to 100 kPa, or from 1 kPa to 100 kPa, or from 1 kPa to 20 kPa, or from 10 kPa to 50 kPa, or from 5 kPa to 20 kPa, or from 10 kPa to 15 kPa, or from 20 kPa to 30 kPa.

Pressure generator 106 may provide an alternating pressure, such that a first period of non-atmospheric pressure is followed by a second period of atmospheric pressure. In one aspect, the first period of the non-atmospheric pressure may be a period with a pressure higher than the atmospheric pressure (hyperbaric period). In another aspect, the first period of the non-atmospheric pressure may be a period with pressure lower than the atmospheric pressure (hypobaric period).

The first period of non-atmospheric pressure may have a duration in a range from 0.001 seconds to 10,000 seconds, or from 0.1 seconds to 5000 seconds, or from 1 second to 1000 seconds, or from 1 second to 500 seconds or from 1 second to 100 seconds or from 1 second to 50 seconds or from 1 second to 20 seconds, or from 5 seconds to 15 seconds, or from 1 second to 10 seconds, or from 1 second to 5 seconds, or from 2 seconds to 4 seconds, or from 5 to 10 seconds, or from 5 to 25 seconds, or from 3 to 7 seconds, or from 8 to 14 seconds, or from 10 to 60 seconds, or from 10 to 40 seconds, or from 10 to 30 seconds, or from 10 to 25 seconds, or from 10 to 20 seconds, or from 10 to 16 seconds.

The second period of atmospheric pressure may have a duration in a range from 0.001 seconds to 10,000 seconds, or from 0.1 seconds to 5000 seconds, or from 1 second to 1000 seconds, or from 1 second to 500 seconds, or from 1 second to 100 seconds or from 1 second to 50 seconds or from 1 second to 20 seconds, or from 5 seconds to 15 seconds, or from 1 second to 10 seconds, or from 1 second to 5 seconds, or from 2 seconds to 4 seconds, or from 5 to 10 seconds, or from 5 to 25 seconds, or from 3 to 7 seconds, or from 8 to 14 seconds, or from 10 to 60 seconds, or from 10 to 40 seconds, or from 10 to 30 seconds, or from 10 to 25 seconds, or from 10 to 20 seconds, or from 10 to 16 seconds.

The pressure difference between the pressure in the therapy chamber of the therapy unit 105 and the atmospheric pressure in the first period of non-atmospheric pressure may be in a range from 0.001 kPa to 1000 kPa, from 0.01 kPa to 1000 kPa, or from 0.01 kPa to 100 Pa, or from 0.1 kPa to 100 kPa, or from 1 kPa to 100 kPa, or from 1 kPa to 20 kPa, or from 10 kPa to 50 kPa, or from 5 kPa to 20 kPa, or from 10 kPa to 15 kPa, or from 20 kPa to 30 kPa.

The pressure generator 106 may provide an alternating pressure, such that a first period of non-atmospheric pressure is followed by a second period of non-atmospheric pressure. The first period of non-atmospheric pressure may be a period with a pressure higher than atmospheric pressure and the second period of non-atmospheric pressure may be a period with a pressure higher than atmospheric pressure, wherein the pressure of the second period may be different from the first period of non-atmospheric pressure. In another aspect of the invention, the first period of non-atmospheric pressure may be a period with a pressure lower than atmospheric pressure and the second period of non-atmospheric pressure may be a period with pressure lower than atmospheric pressure, wherein the pressure of the second period may be different from the first period of non-atmospheric pressure.

In one aspect of the invention, the pressure generator 106 may provide an alternating pressure cycle comprising three periods, such that a first period of non-atmospheric pressure is followed by a second period of atmospheric pressure and a third period of non-atmospheric pressure. The first period may have a duration T1, the second period may have duration T2, the third period may have duration T3. The duration of the first period T1 may the be same or different than the duration of the second period T2, the duration of the second period T2 may be the same or different than the duration of the third period T3, the duration of the first period T1 may be the same or different than the duration of the third period T3.

The duration of at least one of T1, T2 or T3 may be in a range from 0.001 seconds to 10,000 seconds, or from 0.1 seconds to 5000 seconds, or from 1 second to 1000 seconds, or from 1 second to 500 seconds, or from 1 second to 100 seconds or from 1 second to 50 seconds or from 1 second to 20 seconds, or from 5 seconds to 15 seconds, or from 1 second to 10 seconds, or from 1 second to 5 seconds, or from 2 seconds to 4 seconds, or from 5 to 10 seconds, or from 5 to 25 seconds, or from 3 to 7 seconds, or from 8 to 14 seconds, or from 10 to 60 seconds, or from 10 to 40 seconds, or from 10 to 30 seconds, or from 10 to 25 seconds, or from 10 to 20 seconds, or from 10 to 16 seconds.

In one aspect, the non-atmospheric pressure in the first period and the third period may be higher than atmospheric pressure. In another aspect, the non-atmospheric pressure in the first period and the third period may be lower than atmospheric pressure. The pressure in the first period may be the same or different than the pressure in the third period.

The pressure difference between the pressure in the therapy chamber of the therapy unit 105 and the atmospheric pressure in the first period of non-atmospheric pressure or in the third period of non-atmospheric pressure may be in a range from 0.001 kPa to 1000 kPa, from 0.01 kPa to 1000 kPa, or from 0.01 kPa to 100 Pa, or from 0.1 kPa to 100 kPa, or from 1 kPa to 100 kPa, or from 1 kPa to 20 kPa, or from 10 kPa to 50 kPa, or from 5 kPa to 20 kPa, or from 10 kPa to 15 kPa, or from 20 kPa to 30 kPa.

In one non-limiting example, the first period of non-atmospheric pressure lasts for the duration of T1 and the third period of non-atmospheric pressure lasts for the third period T3, wherein T1 is equal to T3. In one aspect the pressure of the first period of non-atmospheric pressure is higher than the pressure of the third period of non-atmospheric pressure. In another aspect, the pressure of the first period of non-atmospheric pressure is lower than the pressure of the third period of non-atmospheric pressure.

In another non-limiting example, the first period of non-atmospheric pressure lasts for the duration of T1 and the third period of non-atmospheric pressure lasts for the third period T3, wherein T1 is different than T3. In one aspect the pressure of the first period of non-atmospheric pressure is higher than the pressure of the third period of non-atmospheric pressure and T1 is shorter than T3. In another aspect, the pressure of the first period of non-atmospheric pressure is lower than the pressure of the third period of non-atmospheric pressure and T1 is shorter than T3. In another aspect, the pressure of the first period of non-atmospheric pressure is higher than the pressure of the third period of non-atmospheric pressure and T1 is longer than T3. In yet another aspect, the pressure of the first period of non-atmospheric pressure is lower than the pressure of the third period of non-atmospheric pressure and T1 is longer than T3.

In another aspect of the invention, the pressure generator 106 may provide an alternating pressure cycle comprising four periods, such that a first period of non-atmospheric pressure is followed by a second period of atmospheric pressure, a third period of non-atmospheric pressure and a fourth period of atmospheric pressure. The first period may have a duration T1, the second period may have duration T2, the third period may have duration T3, the fourth period may have duration T4.

The duration of the first period T1 may be the same or different than T2, T3 or T4. The duration of the second period T2 may be the same or different than T1, T3 or T4. The duration of the third period T3 may be the same or different than T1, T2, or T4. The duration of the fourth period T4 may be the same or different than T1, T2, or T3.

The duration of at least one of T1, T2, T3 or T4 may be in a range from 0.001 seconds to 10,000 seconds, or from 0.1 seconds to 5000 seconds, or from 1 second to 1000 seconds, or from 1 second to 500 seconds, or from 1 second to 100 seconds or from 1 second to 50 seconds or from 1 second to 20 seconds, or from 5 seconds to 15 seconds, or from 1 second to 10 seconds, or from 1 second to 5 seconds, or from 2 seconds to 4 seconds, or from 5 to 10 seconds, or from 5 to 25 seconds, or from 3 to 7 seconds, or from 8 to 14 seconds, or from 10 to 60 seconds, or from 10 to 40 seconds, or from 10 to 30 seconds, or from 10 to 25 seconds, or from 10 to 20 seconds, or from 10 to 16 seconds.

In one aspect, the non-atmospheric pressure in the first period and third period may be higher than atmospheric pressure. In another aspect, the non-atmospheric pressure in the first period and third period may be lower than atmospheric pressure. The pressure in the first period may be the same or different than the pressure in the third period.

The pressure difference between the pressure in the therapy chamber of the therapy unit 105 and the atmospheric pressure in the first period of non-atmospheric pressure or in the third period of non-atmospheric pressure may be in a range from 0.001 kPa to 1000 kPa, from 0.01 kPa to 1000 kPa, or from 0.01 kPa to 100 Pa, or from 0.1 kPa to 100 kPa, or from 1 kPa to 100 kPa, or from 1 kPa to 20 kPa, or from 10 kPa to 50 kPa, or from 5 kPa to 20 kPa, or from 10 kPa to 15 kPa, or from 20 kPa to 30 kPa.

The alternating pressure provided by the pressure generator 106 may have a profile that characterizes the pressure changes. The profile may be a square profile, a triangle profile, a ramp profile, a descending ramp profile, an exponential profile, an exponential decay profile, a trapeze profile and others. The profile may comprise a ramp-up time, a constant time and a ramp-down time. The alternating pressure provided by the pressure generator 106 may comprise an atmospheric pressure period. The atmospheric pressure period may alternate with non-atmospheric pressure period.

The pressure of the first period may be the same or different than the pressure of the second period. In one aspect of the invention, the duration of the first period may be the same as the duration of the second period. In another aspect of the invention, the duration of the first period may be different than the duration of the second period.

In one aspect of the invention, the device may provide more than one period of non-atmospheric pressure in a sequential manner. In one aspect of the invention, the device may provide more than one period of non-atmospheric pressure and more than one period of atmospheric pressure in a sequential manner.

Pressure generator 106 may be in communication with the main unit 101, the power supply 102, the control unit 103, the user interface 104, the therapy unit 105, and at least one sensor 107. At least one sensor 107 may provide feedback about the state of the therapy unit 105 to the control unit 103. The feedback may be used to adjust the parameters of the pressure generator 106.

The state of therapy unit 105 may include, for example, the pressure measured in therapy unit 105, the pressure measured in the sealing cuff, the measured flow of the air, the measured position of therapy unit 105, and/or others.

In one aspect of the invention, the pressure generator 106 may provide alternating pressure in the form of a pressure cycle. At the beginning of the pressure cycle, the pressure in the therapy chamber 201 may be the same as the atmospheric pressure. In the first part of the pressure cycle, the pressure in the therapy chamber 201 may continually increase until a positive pressure threshold is reached. In the second part of the pressure cycle, the pressure in the therapy chamber 201 may continually decrease, until a negative pressure threshold is reached. In the third part of the pressure cycle, the pressure in the therapy chamber 201 may continually increase, until the pressure in the therapy chamber 201 is the same as atmospheric pressure.

In another aspect of the invention, the pressure generator 106 may provide alternating pressure in the form of a pressure cycle. At the beginning of the pressure cycle, the pressure in the therapy chamber 201 may be the same as the atmospheric pressure. In the first part of the pressure cycle, the pressure in the therapy chamber 201 may continually increase until a positive pressure threshold is reached. In the second part of the pressure cycle, the pressure in the therapy chamber 201 may continually decrease until the pressure in the therapy chamber 201 is the same as atmospheric pressure.

In another aspect of the invention, the pressure generator 106 may provide alternating pressure in the form of a pressure cycle. At the beginning of the pressure cycle, the pressure in the therapy chamber 201 may be the same as the atmospheric pressure. In the first part of the pressure cycle, the pressure in the therapy chamber 201 may continually decrease until a negative pressure threshold is reached. In the second part of the pressure cycle, the pressure in the therapy chamber 201 may continually increase until the pressure in the therapy chamber 201 is the same as atmospheric pressure.

The pressure generator 106 may introduce changes in the pressure in the therapy unit 105. The pressure changes may be in a range from 0.001 kPa/second to 1000 kPa/second, or from 0.001 kPa/second to 100 kPa/second, 0.001 kPa/second to 10 kPa/second, or from 0.001 kPa/second to 5 kPa/second or from 0.001 kPa/second to 1 kPa/second, or from 0.01 kPa/second to 10 kPa/second, or from 0.01 kPa/second to 1 kPa/second, or from 0.1 kPa/second to 10 kPa/second, or from 0.1 kPa/second to 1 kPa/second, or from 0.25 kPa/second to 0.5 kPa/second.

Pressure generator 106 may be a blower. The blower may be used to alternate the pressure in therapy unit 105. The blower may be communicating with the control unit 103 and/or at least one sensor 107. The blower may have an airflow rate in a range from 0.001 L/minute to 10,000 L/minute, or from 1 L/minute to 10,000 L/minute, or from 1 L/minute to 5000 L/minute, or from 50 L/minute to 1000 L/minute or from 100 L/minute to 1000 L/minute or from 300 L/minute to 800 L/minute.

The relationship between the speed of gas inflow and/or outflow and changing pressure may be that an inflow speed of about 1 m/s is able to change the pressure in the therapy chamber 201 in the range of, for example, about 20 to about 500 mmHg. The speed of gas inflow and/or outflow may be changed and regulated during therapy.

The blower may induce pressure changes in the therapy unit 105. The pressure changes may be from 0.001 kPa to 1000 kPa, from 0.01 kPa to 500 kPa, or from 0.01 kPa to 200 Pa, or from 0.1 kPa to 100 kPa, or from 1 kPa to 100 kPa, or from 1 kPa to 20 kPa, or from 1 kPa to 50 kPa, or from 10 kPa to 50 kPa, or from 5 kPa to 20 kPa, or from 10 kPa to 15 kPa, or from 20 kPa to 30 kPa.

The blower may be able to regulate the pressure changes very quickly. The blower may induce changes in the pressure in the therapy unit 105 of the duration in a range from 0.001 seconds to 1000 seconds, or from 0.1 seconds to 1000 seconds, or from 1 second to 1000 seconds, or from 1 second to 500 seconds or from 1 second to 100 seconds or from 1 second to 50 seconds or from 1 second to 20 seconds, or from 5 seconds to 15 seconds, or from 1 second to 5 seconds. The fast pressure changes introduced by the blower may be beneficial for vacuum compression therapy (pressure therapy). The blower may be able to introduce a high-pressure environment in the therapy chamber 201 as well as a low-pressure environment.

The blower may induce changes in the pressure in the therapy unit 105. The pressure changes may be in a range from 0.001 kPa/second to 1000 kPa/second, or from 0.001 kPa/second to 100 kPa/second, or from 0.01 kPa/second to 100 kPa/second, or from 1 kPa/second to 50 kPa/second, or from 1 kPa/second to 10 kPa/second, or from 2 kPa/second to 6 kPa/second, or from 2.5 kPa/second to 5 kPa/second.

The blower may comprise a proportional pressure regulator. The proportional pressure regulator may be in communication with the control unit 103 and may provide feedback about the state of the therapy unit 105 to the control unit 103. The state of the therapy unit 105 may be for example the pressure in the therapy unit or the quality of the sealing. The proportional pressure regulator may comprise at least one sensor 107. At least one sensor 107 may be a pressure sensor and/or a flow sensor. At least one sensor 107, may be in communication with the control unit 103 and may provide feedback about the state of the therapy unit 105 to the control unit 103. The proportional pressure regulator may be able to provide very fast feedback about the state of the therapy unit 105.

The blower may provide an alternating pressure, such that a first period is followed by a second period. The blower may comprise an alternating pressure rate that describes the number of times the pressure is altered per unit of time. The blower may have an alternating pressure rate from 0.0001 Hz to 1000 Hz, or from 0.001 Hz to 100 Hz, or from 0.01 Hz to 100 Hz, or from 0.1 Hz to 100 Hz, or from 1 Hz to 100 Hz, or from 0.001 Hz to 50 Hz, or from 0.001 Hz to 10 Hz, or from 0.001 Hz to 1 Hz, or from 0.01 Hz to 100 Hz, or from 0.01 Hz to 10 Hz, or from 0.01 Hz to 1 Hz, or from 0.05 Hz to 1 Hz, or from 0.1 Hz to 1 Hz.

The blower may provide a first period of pressure and a second period of pressure. The first and the second periods of pressure may be non-atmospheric pressures. The pressures of the first period and the second period may be different. In one aspect, the pressure of the first period may be higher than the atmospheric pressure and the pressure of the second period may be lower than the atmospheric pressure or vice versa. In another aspect, the pressure of the first period may be higher than the atmospheric pressure and the pressure of the second period may be higher than the atmospheric pressure, different from the atmospheric pressure of the first period. In another aspect, the pressure of the first period may be lower than the atmospheric pressure and the pressure of the second period may be lower than the atmospheric pressure, different from the atmospheric pressure of the first period. In yet another aspect, the pressure of the first period may be different than the atmospheric pressure and the pressure of the second period may be the same as atmospheric pressure.

The pressure ratio of a pressure generator is the ratio of the pressure generator discharge pressure to the pressure generator inlet pressure. The blower may have a pressure ratio in a range from 1 to 1.2, or from 1.05 to 1.2, or from 1.1 to 1.2, or from 1.11 to 1.2.

In one aspect of the invention, the blower may provide alternating pressure in the form of a pressure cycle. The pressure cycle may comprise a positive pressure threshold and a negative pressure threshold.

The positive pressure threshold of the blower may be defined as the maximal difference between the pressure in the therapy chamber 201 and the atmospheric pressure. The positive pressure threshold may be for example in a range from 0.001 kPa to 1000 kPa, from 0.001 kPa to 500 kPa, or from 0.001 kPa to 200 kPa, or from 0.001 kPa to 100 kPa, or from 1 kPa to −100 kPa, or from 1 kPa to 20 kPa, or from 1 kPa to 50 kPa, or from 1 kPa to 16 kPa, or from 1 kPa to 30 kPa, or from 1 kPa to 10 kPa, or from 0.001 kPa to 50 kPa, or from 0.001 kPa to 20 kPa, or from 0.001 kPa to 10 kPa, or from 0.001 kPa to 16 kPa, or from 10 kPa to 16 kPa, or from 5 kPa to 10 kPa, or from 7 kPa to 9 kPa, or from 9 kPa to 11 kPa, or from 1 kPa to 5 kPa.

The negative pressure threshold of the blower may be defined as the maximal difference between the pressure in the therapy chamber 201 and the atmospheric pressure. The negative pressure threshold may be for example in a range from −0.001 kPa to −1000 kPa, from −0.001 kPa to −500 kPa, or from −0.001 kPa to −200 kPa, or from −0.001 kPa to −100 kPa, or from −1 kPa to −100 kPa, or from −1 kPa to −20 kPa, or from −1 kPa to −50 kPa, or from −1 kPa to −16 kPa, or from −1 kPa to −30 kPa, or from −1 kPa to −10 kPa, or from −0.001 kPa to −50 kPa, or from −0.001 kPa to −20 kPa, or from −0.001 kPa to −10 kPa, or from −0.001 kPa to −16 kPa, or from −10 kPa to −16 kPa, or from −5 kPa to −10 kPa, or from −7 kPa to −9 kPa, or from −9 kPa to −11 kPa, or from −1 kPa to −5 kPa.

The blower may provide an alternating pressure in the form of a pressure cycle. The pressure cycle of the blower may have a duration in a range from 0.001 seconds to 1000 seconds, or from 0.1 seconds to 1000 seconds, or from 1 second to 1000 seconds, or from 1 second to 500 seconds or from 1 second to 100 seconds or from 1 second to 50 seconds or from 1 second to 20 seconds, or from 5 seconds to 15 seconds, or from 10 seconds to 30 seconds, or from 15 seconds to 25 seconds.

In one aspect of the invention, the blower may be configured to provide a pressure cycle that comprises three parts. At the beginning of the pressure cycle, the pressure in the therapy chamber 201 may be the same as the atmospheric pressure. In the first part of the pressure cycle, the pressure in the therapy chamber 201 may continually increase until a positive pressure threshold is reached. In the second part of the pressure cycle, the pressure in the therapy chamber 201 may continually decrease, until a negative pressure threshold is reached. In the third part of the pressure cycle, the pressure in the therapy chamber 201 may continually increase, until the pressure in the therapy chamber 201 is the same as atmospheric pressure.

In one aspect of the invention, the positive pressure threshold is in a range from 10 kPa to 16 kPa and the negative pressure threshold is in a range from −10 kPa to −16 kPa and the duration of the pressure cycle is in a range from 5 to 20 seconds.

In one aspect of the invention, the positive pressure threshold is in a range from 10 kPa to 16 kPa and the negative pressure threshold is in a range from −10 kPa to −16 kPa and the duration of the pressure cycle is in a range from 25 seconds to 25 seconds.

In one aspect of the invention, the positive pressure threshold is in a range from 1 kPa to 50 kPa and the negative pressure threshold is in a range from −1 kPa to −50 kPa and the duration of the pressure cycle is in a range from 5 to 20 seconds.

In one aspect of the invention, the user and/or the operator chooses the intensity of the pressure changes and the negative pressure threshold or the positive pressure threshold.

The intensity of the pressure changes may be, for example, in a range from 0.001 kPa to 1000 kPa, from 0.001 kPa to 500 kPa, or from 0.001 kPa to 200 kPa, or from 0.01 kPa to 200 kPa, or from 0.01 kPa to 100 kPa, or from 0.01 kPa to 50 kPa, or from 0.1 kPa to 50 kPa, or from 1 kPa to 50 kPa, or from 1 kPa to 45 kPa, or from 1 kPa to 45 kPa, or from 35 kPa to 45 kPa, or from 1 kPa to 35 kPa, or from 25 kPa to 35 kPa, or from 1 kPa to 30 kPa, or from 1 kPa to 25 kPa, or from 15 kPa to 25 kPa, or from 1 kPa to 20 kPa, or from 12 kPa to 16 kPa, or from 1 kPa to 15 kPa, or from 1 kPa to 13 kPa, or from 7 kPa to 13 kPa, or from 9 kPa to 11 kPa, or from 1 kPa to 10 kPa, or from 7 kPa to 9 kPa, or from 5 kPa to 7 kPa, or from 1 kPa to 5 kPa.

The pressure generator 106 may be a compressor. The compressor may communicate with the control unit 103 and/or at least one sensor 107. The compressor may induce changes in the pressure in the therapy unit 105; the pressure changes may be in a range from 0.001 kPa/second to 10 kPa/second, or from 0.001 kPa/second to 5 kPa/second or from 0.001 kPa/second to 1 kPa/second, or from 0.01 kPa/second to 10 kPa/second, or from 0.01 kPa/second to 1 kPa/second, or from 0.1 kPa/second to 10 kPa/second, or from 0.1 kPa/second to 1 kPa/second, or from 0.25 kPa/second to 0.5 kPa/second.

The compressor may have an airflow rate in a range from 0.001 L/min to 1000 L/min, or from 0.1 1/min to 500 L/min, or from 0.1 L/min to 100 L/min, or from 1 L/min to 100 L/min, or from 1 L/min to 50 L/min.

The compressor may induce pressure changes in the therapy unit 105.

The pressure changes in the therapy unit 105 may be in a range from 0.001 kPa to 10,000 kPa, or from 0.001 kPa to 1000 kPa, or from 0.01 to 1000 kPa, or from 0.1 kPa to 100 kPa, from 1 kPa to 100 kPa, or from 10 kPa to 50 kPa, or from 10 to 20 kPa.

The compressor may induce changes of the pressure in the therapy unit 105 of the duration in a range from 1 second to 10,000 seconds, or from 1 second to 5000 seconds, or from 1 second to 1000 seconds, or from 1 second to 500 seconds, or from 50 to 200 seconds, or from 75 to 150 seconds, or from 100 to 140 seconds, or from 90 seconds to 200 seconds, or from 90 seconds to 110 seconds, or from 160 seconds to 200 seconds, or from 10 seconds to 50 seconds, or from 40 seconds to 55 seconds.

The compressor may provide an alternating pressure, such that a first period is followed by a second period. The blower may comprise an alternating pressure rate, that describes the number of times the pressure is altered per unit of time. The blower may have an alternating pressure rate from 0.0001 Hz to 1 Hz, or from 0.001 Hz to 1 Hz, or from 0.01 Hz to 100 Hz, or from 0.01 Hz to 10 Hz, or from 0.01 Hz to 1 Hz, or from 0.05 Hz to 1 Hz, or from 0.1 Hz to 1 Hz.

The compressor may have a pressure ratio from 1 to 5, or from 1 to 4, or from 1 to 3, or from 1 to 2.

In one aspect of the invention, the compressor may provide an alternating pressure in the form of a pressure cycle. The pressure cycle may comprise a positive pressure threshold and a negative pressure threshold.

The positive pressure threshold of the compressor may be defined as the maximal difference between the pressure in the therapy chamber 201 and the atmospheric pressure. The positive pressure threshold may be for example in a range from 0.001 kPa to 1000 kPa, from 0.001 kPa to 500 kPa, or from 0.001 kPa to 200 kPa, or from 0.001 kPa to 100 kPa, or from 1 kPa to −100 kPa, or from 1 kPa to 20 kPa, or from 1 kPa to 50 kPa, or from 1 kPa to 16 kPa, or from 1 kPa to 30 kPa, or from 1 kPa to 10 kPa, or from 0.001 kPa to 50 kPa, or from 0.001 kPa to 20 kPa, or from 0.001 kPa to 10 kPa, or from 0.001 kPa to 16 kPa, or from 10 kPa to 16 kPa.

The negative pressure threshold of the compressor may be defined as the maximal difference between the pressure in the therapy chamber 201 and the atmospheric pressure. The negative pressure threshold may be for example in a range from −0.001 kPa to −1000 kPa, from −0.001 kPa to −500 kPa, or from −0.001 kPa to −200 kPa, or from −0.001 kPa to −100 kPa, or from −1 kPa to −100 kPa, or from −1 kPa to −20 kPa, or from −1 kPa to −50 kPa, or from −1 kPa to −16 kPa, or from −1 kPa to −30 kPa, or from −1 kPa to −10 kPa, or from −0.001 kPa to −50 kPa, or from −0.001 kPa to −20 kPa, or from −0.001 kPa to −10 kPa, or from −0.001 kPa to −16 kPa, or from −10 kPa to −16 kPa.

The blower may provide an alternating pressure in the form of a pressure cycle. The pressure cycle of the compressor may have a duration in a range from 1 second to 10,000 seconds, or from 50 seconds to 1000 seconds, or from 100 seconds to 1000 seconds, or from 100 seconds to 750 seconds, or from 100 seconds to 500 seconds, or from 100 seconds to 250 seconds, or from 100 seconds to 200 seconds, or from 100 seconds to 150 seconds, or from, or from 90 seconds to 200 seconds, or from 90 seconds to 110 seconds, or from 160 seconds to 200 seconds.

In one aspect of the invention, the positive pressure threshold is in a range from 10 kPa to 16 kPa and the negative pressure threshold is in a range from −10 kPa to −16 kPa and the duration of the pressure cycle is in a range from 100 to 150 seconds.

In one aspect of the invention, the positive pressure threshold is in a range from 10 kPa to 16 kPa and the negative pressure threshold is in a range from −10 kPa to −16 kPa and the duration of the pressure cycle is in a range from 100 seconds to 250 seconds.

In one aspect of the invention, the positive pressure threshold is in a range from 1 kPa to 50 kPa and the negative pressure threshold is in a range from −1 kPa to −50 kPa and the duration of the pressure cycle is in a range from 100 to 150 seconds.

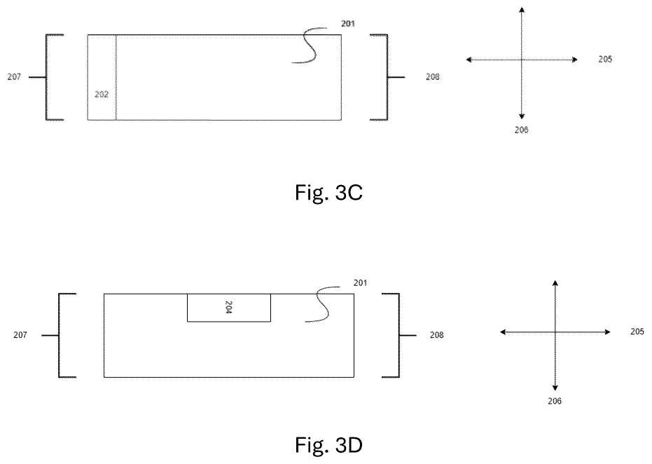

FIGS. 3A-D show exemplary schematic diagrams of the therapy chamber 201.

FIGS. 3A and 3B show an exemplary schematic diagram of the therapy chamber 201. The therapy chamber may have a cuboid shape, a block shape, a cylindrical shape, a semi cuboid shape, a semi block shape, or semi cylindrical shape. The therapy chamber 201 may bend in various angles to provide the best fit for the anatomical body part. The therapy chamber 201 may comprise a first base 207 and a second base 208. The therapy chamber 201 may comprise a lateral surface 209. The therapy chamber 201 may comprise at least one opening 204.

The therapy chamber 201 may comprise a first axis of 205 and a second axis of 206. A length of the therapy chamber 201 may be defined by the first axis 205 and a width defined by the second axis 206. In one aspect the length of the therapy chamber 201 is greater than the width of the therapy chamber 201. In another aspect the length of the therapy chamber 201 is equal to the width of the therapy chamber 201. Yeat in another aspect the width of the therapy chamber 201 is greater than the length of the therapy chamber 201.

At least one opening 204 may be positioned at one end of the therapy chamber 201, in the middle of the therapy chamber 201, in one third of the therapy chamber, or in any other position on the first axis 205 of the therapy chamber 201. At least one opening 204 may have a circular shape, a square shape, a rectangle shape, or arbitrary polygonal shape (e.g. amorphous).

At least one opening 204 may be centralized with the first axis 205 of the therapy chamber 201.

At least one opening 204 may be positioned at any position on the first axis 205 of the therapy chamber 201. For example, the distance from the first base 207 and/or the second base 208 of the therapy chamber 201 to the opening 204 may be in a range from 1 cm to 100 cm, or from 1 cm to 75 cm, or from 15 cm to 50 cm. The distance from the first and/or the second base 208 of the therapy chamber 201 to the opening 204 may be about ⅕ of the overall length of the therapy chamber 201, about ¼ of the overall length, about ⅓ of the overall length, or about ½ of the overall length.

In one aspect, the opening 204 may be positioned at any position on the first axis 205 of the therapy chamber 201 except at the longitudinal ends of the therpy chamber (defined by the ends of the first axis 205).

FIG. 3C shows the location of the sealing unit 202 in the therapy chamber 201. The sealing unit 202 may be located at the first base 207 of the therapy chamber 201, or at the second base 208 of the therapy chamber 201.

The sealing unit 202 may be configured to allow for insertion of the body part into the therapy chamber 202 and create an airtight seal around the body part during the treatment.

In one aspect of the invention, the therapy unit 105 may comprise two sealing units 202. For example, a first sealing unit 202 located at the first base 207 and a second sealing unit 202 located at the second base 208. In another aspect, two sealing units 202 may be arranged behind each other at the first base 207 of the therapy chamber 201 or at the second base 208 of the therapy chamber 201.

The therapy chamber 201 may comprise at least one opening 204. The at least one opening 204 may enable easier access to the therapy chamber 201, easier cleaning and disinfecting of the therapy chamber 201, and a port to add any additional treatment elements 108 to the therapy chamber 201.

FIG. 3D shows the location of at least one opening 204 on the therapy chamber 201. The opening 204 may be located at the lateral surface 209 of the therapy chamber 201.

The therapy chamber 201 may comprise two or more openings 204. The two or more openings may be located anywhere on the therapy chamber 201. The two or more openings 204 may be located on the lateral surface 209 of the therapy chamber 201.

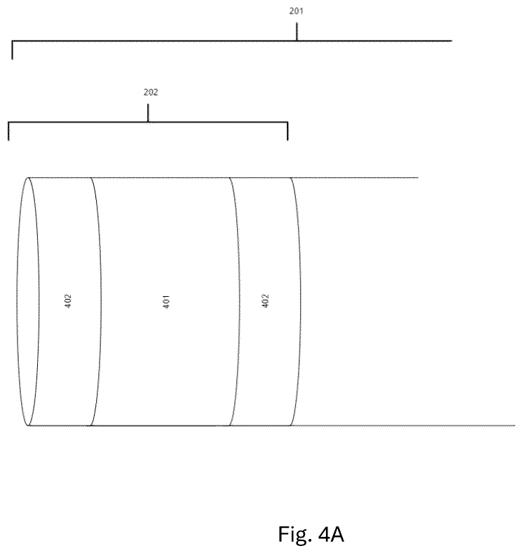

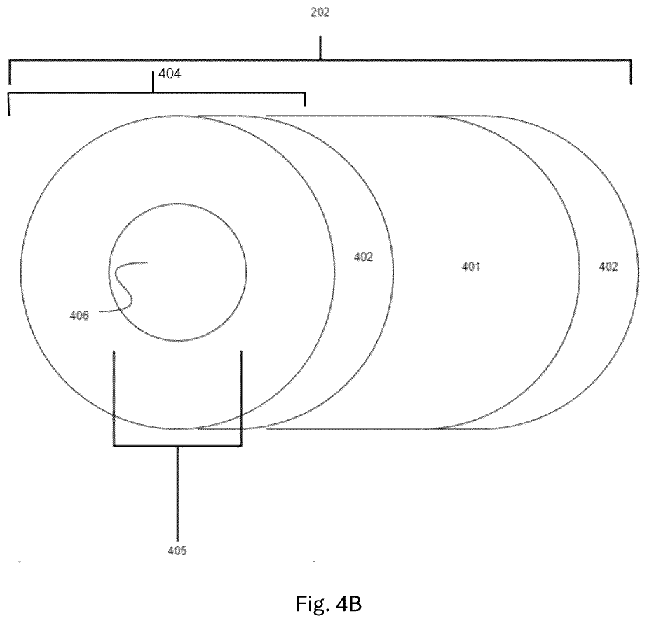

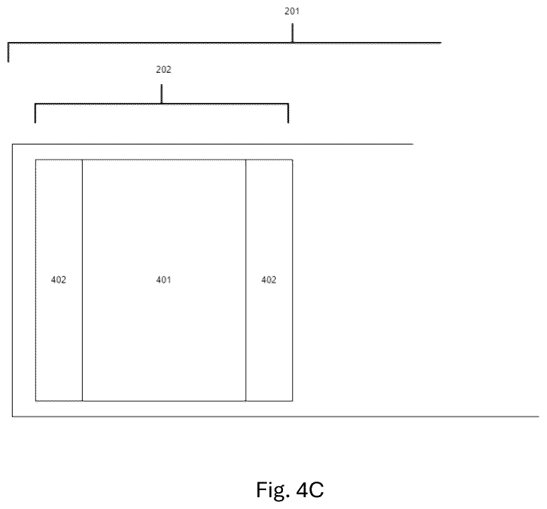

FIGS. 4A-C show exemplary schematic diagrams of the sealing unit 202.

In one aspect, the sealing unit 202 may comprise a cuff 401. The sealing unit 202 may comprise at least one of: a cuff 401, an inner tubular member 402 or a cuff pressure generator. The cuff pressure generator may be the pressure generator 106. The cuff 401 may be an inflatable cuff 401, which creates an airtight seal around the body part. The cuff 401 is deflated prior to the subject inserting their body part into the sealing unit 202. After the subject inserts their body part into the sealing unit 202, the cuff 401 is inflated (e.g. by a cuff pressure generator), and the body part is sealed.

The cuff 401 may change its volume based on its state, when the cuff is inflated its volume may be larger than when it is deflated. The volume of the cuff 401 may be in a range from 0.01 L to 100 L, or from 0.05 L to 50 L, or from 0.1 L to 10 L, or from 0.5 L to 5 L.

The ratio of the volume of the cuff in its inflated state versus its deflated state may be in a range from 1 to 100, or from 1 to 50, or from 1 to 20, or from 1 to 10, or from 1 to 5, or from 1 to 2.5, or from 1 to 2, or from 1 to 1.5, or from 1 to 1.3, or from 1 to 1.2, or from 1 to 1.1, or from 1.1 to 2.

In another aspect, the ratio of the volume of the cuff in the inflated and deflated state may be in the range of 1 to 100, or 1 to 50, or 1 to 20, or 1 to 10, or 1 to 5, or 1 to 2.5, or 1 to 2, or 1 to 1.5, or 1 to 1.3, or 1 to 1.2, or 1 to 1.1, or 1.1 to 2, not including the extremes of the ranges.

The cuff 401 may be releasably coupled to the therapy chamber 201 at the first base 207 and/or the second base 208. The therapy chamber 201 may also include at least one safety feature, such as an inner tubular member 402, that ensures the cuff 401 stays in the right position and creates an airtight seal around the body part.

In one aspect, the therapy chamber 201 comprises two inner tubular members 402 configured to sandwich the cuff 401 from two sides in the therapy chamber 201 at the first base 207 or the second base 208, as schematically shown in FIG. 4A.

The cuff 401 may be formed inside the therapy chamber 201. The cuff 401 may have a shape of the therapy chamber 201.

In one exemplary case, the cuff 401 may be formed inside the cylindrical therapy chamber 201. The cuff 401 may have a cylindrical shape. The cuff 401 may include a valve for inflating and deflating the cuff 401 (e.g. by cuff pressure generator). The valve communicate with control unit 103. The cuff 401 may be inflated or deflated prior to the start of the therapy, during the therapy, or after the therapy. The cuff 401 may also be inflated or deflated based on information acquired by at least one sensor 107. The cuff 401 may also be inflated or deflated when a safety risk is detected by a control unit 103.

The cuff 401 may have a cylindrical shape, a tubular shape, or a circular cross-section, or any other appropriate shape.

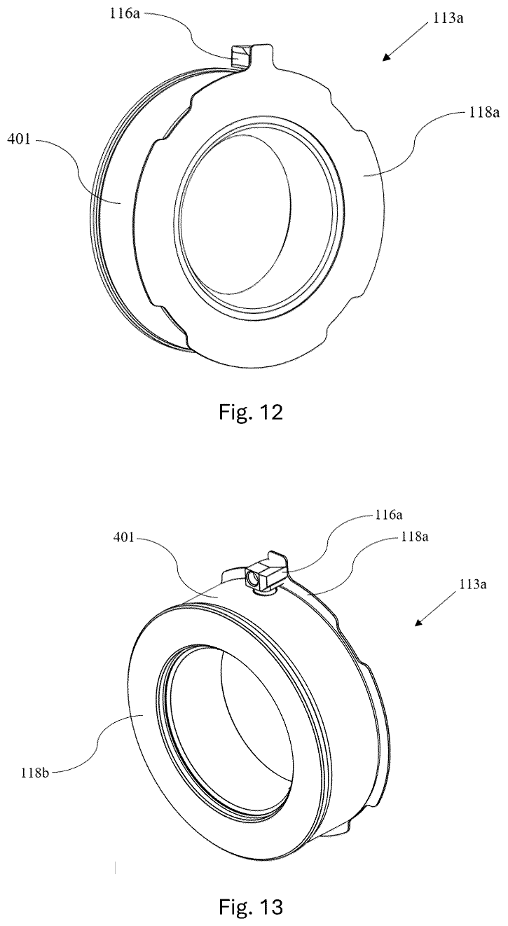

In one aspect of the invention, the cuff 401 may be releasably coupled to the therapy chamber 201 using a click mechanism 113, as depicted in FIGS. 12, 13, 14 and 15.

The click mechanism 113 may comprise a first part 113a of the click mechanism and a second part 113b of the click mechanism.

FIG. 12 depicts a front view of a first part 113a of the click mechanism comprising the cuff 401 equipped with a first part 116a of a cuff connector and a first closing member 118a.

FIG. 13 depicts a back view of a first part 113a of the click mechanism comprising the cuff 401 equipped with a first part 116a of a cuff connector and two closing members, a first closing member 118a and a second closing member 118b.

FIG. 14 depicts a view of the mechanics of a second part 113b of a click mechanism without an inserted cuff 401. The second part 113b of the click mechanism comprises the second part 116b of the cuff connector.

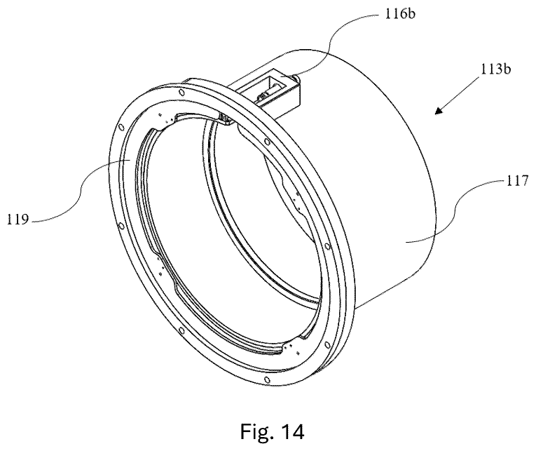

FIG. 15 depicts a view of the mechanics of both parts 113a and 113b of the click mechanism 113 connected together.

The click mechanism 113 may comprise a cuff connector 116.

In one aspect the first part 113a of the click mechanism may comprise a first part 116a of the cuff connector and a second part 113b of the click mechanism may comprise a second part 116b of the cuff connector.

The second part 113b of click mechanism may comprise an inserting member 117 for inserting the cuff 401, the second part 116b of cuff connector for air tight connection of the cuff 401, when the cuff 401 may be inserted into the inserting member 117. The second part 116b of the cuff connector may be connected to a first part 116a of the cuff connector (e.g. a valve) of the cuff 401. The cuff connector 116 (e.g. a first part of the cuff connector 116a or a second part of the cuff connector 116b) may be connected to the pressure generator 106 and/or to the pressure sensor. The cuff connector 116 (e.g. a first part of the cuff connector 116a or a second part of the cuff connector 116b) may be connected both to the pressure generator 106 and the pressure sensor using a “T” shaped member, for example T-piece, T-coupling member or T-tube.

In one aspect, the first part 116a of the cuff connector may comprise the “T” shape member. In one aspect, the second part 116b of the cuff connector may comprise the “T” shape member.

The click mechanism 113 (e.g. first part of 113a click mechanism) may comprise the closing member (e.g. first closing member 118a or second closing member 118b) for closing the cuff 401 into the inserting member 117 and a sleeve 119 for connecting with the closing member and a tight closure of the cuff 401 into the click mechanism 113. The cuff 401 may be placed into the click mechanism 113 for example by turning the sleeve 119 relative to the first closing member 118a, by clicking the first closing member 118a into the sleeve 119, or by any other appropriate way of connecting the first closing member 118a with the sleeve 119. The first closing member 118a or the second closing member 118b may be releasably or non releasably connected to the cuff 401, or may be integrated with the cuff 401. The click mechanism 113 may comprise two closing members, the first closing member 118a (e.g. front closing member) and a second closing member 118b (e.g. back closing member) placed on both sides of the cuff 401, as depicted in FIG. 13. The closing member may prevent the cuff from being released from the click mechanism 113 and also from stretching the cuff 401 sideways during its inflation and allowing inflation and stretching the cuff 401 in a requested direction towards the body part, e.g. limb of the subject of the therapy. The cuff connector 116 may comprise two parts, the first part 116a of cuff connector and the second part 116b of cuff connector.

In one aspect, the second part 113b of the click mechanism may be a part of the therapy unit 105 or may be coupled to the therapy unit 105, and the first part 113a of the click mechanism may be removable. The cuff connector 116 may comprise a first part 116a of a cuff connector coupled to the cuff 401 and a second part 116b of a cuff connector coupled to the second part 113b of the click mechanism.

Some parts of the click mechanism 113, for example the inserting member 117, the closing member or the sleeve 119 may be made from metal, plastic, glass, poly(methyl methacrylate), PMMA, other polymers and/or other rigid materials, or any other appropriate material.

The sealing unit 202 comprising the cuff 401 may be releasably or non releasably coupled to the therapy chamber 201 using at least one cuff release barrier (e.g. tubular member 402 or first meber 118a or second member 118b). The cuff release barrier may have a planar circular shape, or any other appropriate shape. The cuff release barrier may be made from metal, plastic, glass, rubber, poly(methyl methacrylate), PMMA, other polymers and/or other rigid materials, or any other appropriate material. The cuff release barrier may have function to attach the cuff 401 in the therapy chamber and prevent it from being released or falling out of the therapy chamber 201 during the therapy. The cuff release barrier may comprise a planar circular shaped metal component, a rubber pin or rods, or any other appropriate barrier. The cuff release barrier may be placed on at least one side of the cuff 401.

The cuff release barrier may be connected to the therapy chamber 201 using for example at least one of: a latch, screws, bayonet mechanism or click mechanism, or any other appropriate connecting mechanism.

The cuff 401 may be made from flexible and/or elastic material. The cuff 401 may be made from polymer, polyurethane, neoprene rubber, silicone, latex, and others. The inflatable cuff 401 may be inflated using a gas, such as air or oxygen.

The cuff 401 may be made out of a cuff material. The cuff material may have appropriate features to ensure airtight sealing. One of such features may be a tensile strength in a range from 0.01 MPa to 300 MPa, or from 0.1 MPa to 200 MPa, or from 0.1 MPa to 100 MPa, or from 0.1 MPa to 50 MPa, or from 1 MPa to 50 MPa, or from 10 MPa to 30 MPa.

In one aspect, the cuff material may have flexibility (possible elongation) in a range from 1% to 700%, or from 1% to 200%, or from 1% to 100%, or from 200% to 600%, or from 400% to 500%, or from 425% to 475%.

In one aspect, the hardness of the cuff material may be in a range from 0 A to 100 A, from 5 A to 70 A, from 5 A to 50 A, or from 10 A to 50 A, or from 15 A to 45 A, or from 20 A to 40 A, or from 25 A to 35 A.

In one aspect, the cuff material may be resistant to high temperatures. The material may have excellent thermal resistance.

The cuff 401 may be communicating with the cuff pressure generator. In one aspect, the cuff pressure generator and the pressure generator 106 may be the same pressure generator. In another aspect, the cuff pressure generator may be an extra generator that inflates and/or deflates the cuff 401.

The cuff 401 and the cuff pressure generator may be in communication with the main unit 101, with the power supply 102, with the control unit 103 with the user interface 104, with the therapy unit 105, with the pressure generator 106, and/or with the at least one sensor 107.

The cuff 401 may comprise an outer diameter 404 and an inner diameter 405. The device may comprise at least two cuffs 401 with various inner diameter 405 sizes. The most suitable cuff 401 may be chosen and changed prior to the therapy. The cuff 401 may be chosen based on the diameter of the subject's body part. The cuff 401, with an inner diameter 405 that ensures an airtight sealing of the subject's body part, may be used for the therapy.

The cuff 401 may have an inner diameter 405 in a range from 1 cm to 100 cm, or from 2 cm to 50 cm, or from 5 cm to 25 cm, or from 6 cm to 22 cm.

The cuff 401 may have an outer diameter 404 in a range from 1 cm to 100 cm, or from 2 cm to 50 cm, or from 15 to 75 cm, or from 25 to 75 cm, or from 15 to 45 cm, or from 6 cm to 26 cm, or from 5 cm to 25 cm, or from 8 cm to 22 cm, or from 7 cm to 21 cm, or from 100 cm to 150 cm, or from 50 cm to 100 cm, or from 25 cm to 50 cm, or from 26 cm to 50 cm, or from 22 cm to 45 cm, or from 21 cm to 40 cm.

The cuff pressure generator may be a pump, a fan, a compressor, or a blower.

The pressure difference between the pressure in the in the cuff 401 and atmospheric pressure may be in a range from 0 kPa to 100 kPa, from 0.001 kPa to 90 kPa, from 0.01 kPa to 80 kPa, or from 0.1 kPa to 70 kPa, or from 0.5 kPa to 60 kPa, or from 1 kPa to 50 kPa, or from 2 kPa to 48 kPa, or from 5 kPa to 45 kPa, or from 10 kPa to 40 kPa, or from 15 kPa to 35 kPa.

In one aspect of the invention, the pressure in the cuff 401 may be higher than the pressure in the therapy unit 105. The pressure difference between the pressure in the in the cuff 401 and the pressure in the therapy unit 105, when the pressure in the cuff 401 is higher than pressure in the therapy unit 105, may be in a range from 0 kPa to 15 kPa, or from 0.001 kPa to 10 kPa, or from 0.01 kPa to 8 kPa, or from 0.1 kPa to 7 kPa, or from 0.5 kPa to 6 kPa, or from 1 kPa to 5 kPa, or from 2 kPa to 4 kPa. The pressure in the cuff 401 may be higher than the pressure in the therapy unit 105 by the above mentioned ranges of values even in the case that the pressure in the therapy unit 105 is lower than atmospheric pressure.

The cuff 401 may also comprise an aperture 406. The body part may be inserted in the therapy chamber 201 through the aperture 406. After the body part is in the right place in the therapy chamber 201, the cuff 401 may be inflated and a seal between the body part and the cuff 401 is created.

The cuff 401 may be in communication with at least one sensor 107. The at least one sensor 107 may be a pressure sensor that monitors the pressures generated in the cuff 401. The pressure sensor may be connected to the cuff 401 in the place of the gas valve, for example using a T-shaped tube or hose. The pressure sensor and the gas valve may be connected to the cuff 401 through one common opening in the cuff 401 for both components. The at least one sensor 107 may be a flow sensor, that monitors the flow of gas on the at least one gas valve. The at least one gas valve may comprise the input and the output of the therapy chamber 201. The input and the output of the therapy chamber 201 may be the same element. The at least one sensor 107 may provide information on the state of the sealing unit 202 and on the quality of the airtight seal.

The pressure in the cuff 401 may be controlled by the control unit 103. The pressure in the cuff 401 may be periodically controlled. The pressure in the cuff 401 may change during the treatment to provide optimal sealing of the body part.

In one aspect, the cuff 401 may be a folded cuff 512. The sealing unit may comprise 202 at least one folded cuff 512.

A material of the folded cuff 512 may have a large surface, which may provide an advanced airtight seal in comparison with a regular straight cuff 401.

In one aspect the folded cuff 512 may be inflated, the larger surface may enable a very precise sealing of the body part. The folded cuff may be inflated using a gas, such as air or oxygen. The folded cuff may communicate with the cuff pressure generator.

The folded cuff 512 may be made from flexible and/or elastic material. The folded cuff may be made from polymer, polyurethane, neoprene rubber, silicone, latex, and others.

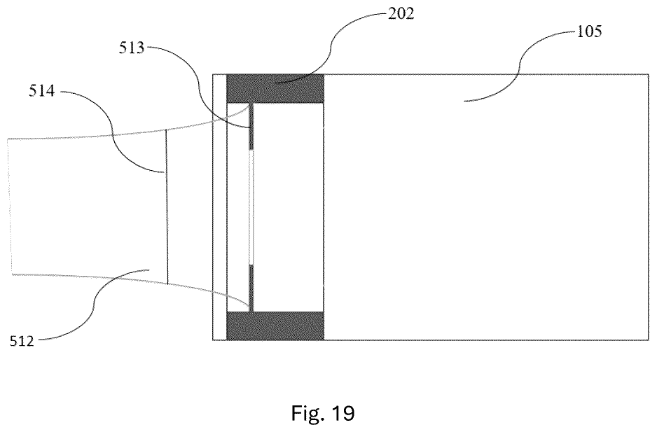

In one aspect of the invention the sealing unit 202 may comprise one folded cuff 512, as shown in FIG. 19.

The folded cuff 512 may comprise a sleeve. The folded cuff 512 may comprise an insert 513 for reducing the diameter of the aperture for inserting the body part, e.g. the limb of the subject of the therapy. The insert 513 may comprise a rigid material.

The folded cuff 512 may comprise a sealing element 514 placed around the folded cuff 512 for better sealing of the body part, e.g. the limb of the subject of the therapy in the folded cuff 512.

The sealing element 514 may comprise Velcro (hook and loop fasteners), snaps, buckles, zippers, hooks and eyes, magnets, elastic bands or straps, adhesive strips, silicone grip strips, buttons and loops, drawstrings or laces, twist-lock or cam-lock closures, ratchet straps, Boa® closure systems, or reusable hook-and-grip systems, or any other system known in the art.

In one aspect the folded cuff 512 may be positioned outside the therapy unit 105, as shown in FIG. 19, which is suitable for therapy using only one direction of pressure changes (e.g. negative pressure therapy). In another aspect, the folded cuff 512 may be positioned intside the therapy unit 105, which is suitable for therapy using only one direction of pressure changes (e.g. positive pressure therapy).

In one aspect, when the body part is inserted into the folded cuff 512, the folded cuff 512 is then folded inward inside the therapy unit 105. Thus one part comprising 10% to 90% or 20% to 80% or 50% to 75% of the folded cuff is positioned outside the therapy unit 105 and the other part is inside the therapy unit 105. Both parts then can be sealed by the sealing elements, respectively. In this aspect, when the folded cuff 512 is at least partially folded inwards, the folded cuff 512 may allow a proper airtight sealing of the body part of the subject of the therapy in both negative and positive pressures relative to the atmospheric pressure.

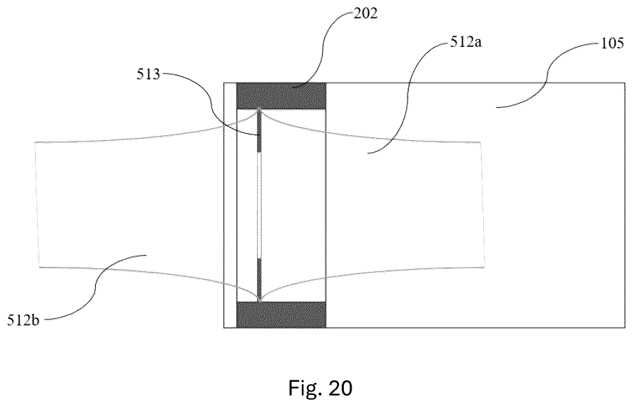

In another aspect of the invention, the sealing unit 202 may comprise at least two folded cuffs 512, e.g. a first folded cuff 512a and a second folded cuff 512b, as shown in FIG. 20. In this aspect two folded cuffs 512 may be connected to the sealing unit 202 and may be positioned in opposite directions, wherein the first folded cuff 512 may be directed inward of the therapy unit 105 and the second folded cuff 512 may be directed outward of the therapy unit 105.

In this aspect, where the sealing unit 202 comprises two folded cuffs 512, the presence of two folded cuffs 512 may allow a proper airtight sealing of the body part, e.g. limb of the subject of the therapy in both negative and positive pressures relative to the atmospheric pressure. Each folded cuff 512 may comprise a sealing element 514.

The sealing unit 202 may comprise an adjustable aperture that is formed by any adjustable sealing mechanism. The adjustable aperture may have an adjustable diameter that can be adjusted to airtightly seal the body parts of various diameter sizes.

The adjustable aperture may have a diameter in a range from 1 cm to 100 cm or from 2 cm to 50 cm, or from 5 cm to 25 cm, or from 6 cm to 22 cm. The diameter of the aperture may vary prior to the therapy/during the therapy, and/or after the therapy. The adjustable aperture and/or the diameter of the adjustable aperture may vary during the therapy, as the control unit may be in communication with the aperture, and the diameter of the aperture may be adjusted based on the feedback provided by at least one sensor 107.

The adjustable sealing mechanism may provide various benefits to the subject, the operator of the device, and the vacuum compression therapy (pressure therapy). The adjustable diameter of the sealing mechanism may enable the use of one sealing mechanism for all diameters of the subject's body part. The adjustable sealing mechanism may reduce the time needed to prepare the device prior to the therapy, as there is no need to use various sealing mechanisms for various body part's diameters. The adjustable sealing mechanism may also be very easy for the operator to use. The adjustable sealing mechanism may also enable the insertion of a body part that wears other means, such as gypsum, splint, screws, and others.

The sealing unit 202 may comprise for example a moldable membrane or a flexible membrane.

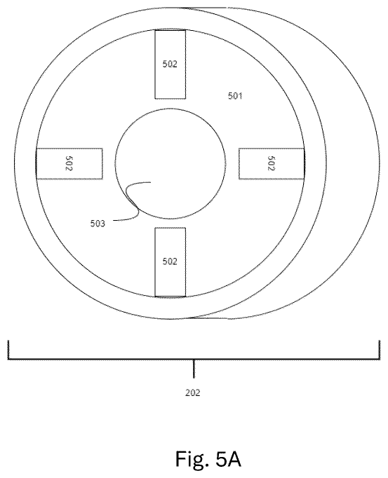

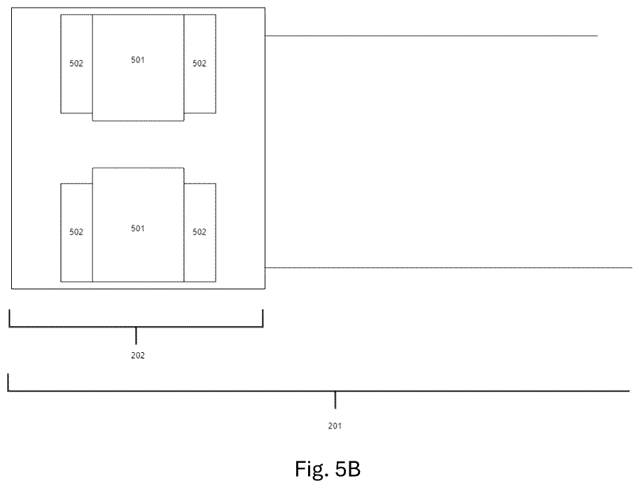

FIGS. 5A-B show additional exemplary schematic diagrams of the sealing unit 202.

In one aspect of the invention, the sealing unit 202 may comprise a moldable membrane 501 and at least one actuator 502. The components of the sealing unit 202 may form an aperture 503. The aperture 503 is formed by the moldable membrane 501 and has an adjustable size. At least one actuator 502 may be hydraulic, pneumatic, or electric. At least one actuator 502 may comprise at least one piston, and/or motor, and/or servo motor and/or liner actuator and/or rotary actuator. A plurality of actuators may be arranged in a circular manner, meaning that the actuators are equally or variably spaced around a central axis, e.g. the actuators are arranged in a circle around the aperture 503.

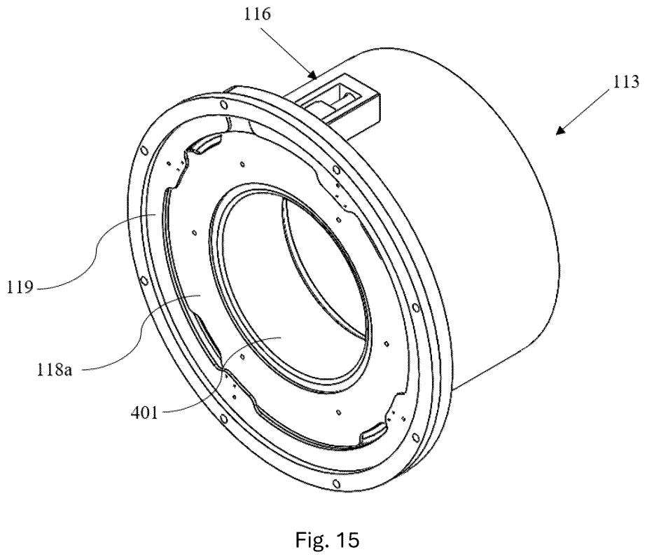

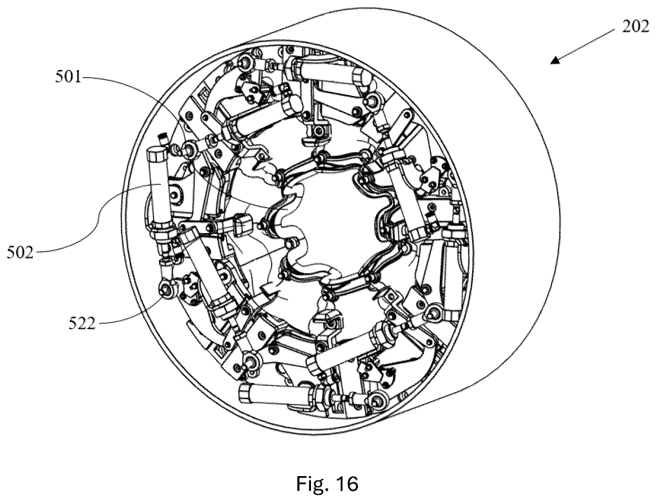

In another aspect of the invention, the sealing unit 202 may be as depicted in FIG. 16. The sealing unit 202 in this aspect may comprise a moldable membrane 501 and at least one actuator 502.

The moldable membrane 501 may be made from flexible and/or elastic material. The moldable membrane 501 may be made from silicone, latex, natural latex, organic latex, synthetic latex, blended latex, neoprene, rubber, polyurethane, polymer, or any other appropriate material.

The moldable membrane 501 may move in order to adjust the size of the adjustable aperture 503 in the sealing unit 202 using a mechanical deformation of the moldable membrane 501. The adjustable aperture 503 may have an adjustable diameter that may be adjusted to airtightly seal the body parts of various diameters and sizes.

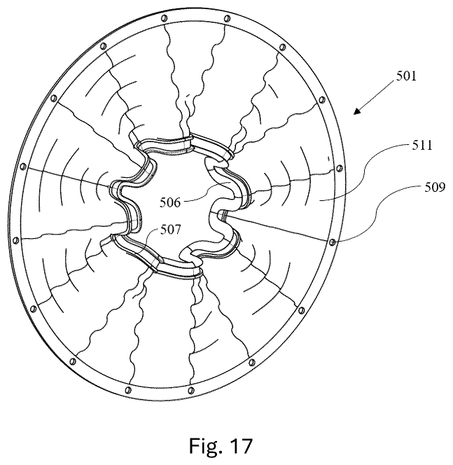

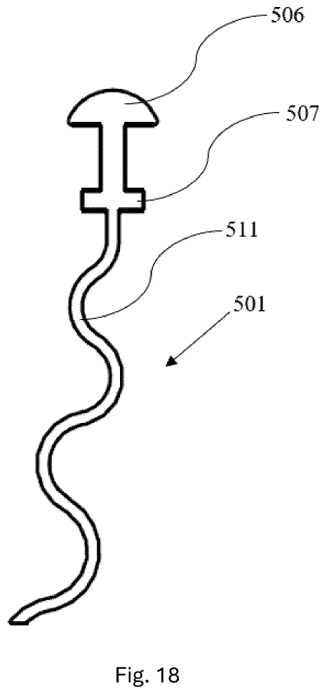

FIG. 17 shows the moldable membrane. FIG. 18 shows a cross-sectional view of the moldable membrane 501. The moldable membrane 501 may comprise a facet 506. The facet 506 is the part of the moldable membrane that may be in contact with the body part, e.g. a limb of the subject of the therapy. The facet 506 may have a width greater than a width of the body 511 of the moldable membrane. The facet 506 may have a rounded shape in order to avoid irritation of the skin or the tissue of the body part, e.g. a limb of the subject of the therapy. The facet 506 may have a shape without sharp angles or without sharp edges. The moldable membrane 501 may comprise a membrane protrusion 507. The membrane protrusion 507 may serve as a member for attaching the moldable membrane 501 and its coupling to the at least one actuator 502. The moldable membrane 501 may comprise at least one pin (not shown in FIGS. 17 and 18), that may also serve as a member for attaching the moldable membrane 501. The pin may be made from a rigid material, e.g. metal, or any other appropriate material.

The moldable membrane 501 may comprise at least one holding point (not shown in FIGS. 17 and 18). The at least one holding point may be situated next to the membrane facet 506, or next to the membrane protrusion 507. The at least one holding point may serve for better body part (e.g. limb) anchoring and positioning. The facet 506 may serve to seal the moldable membrane 501 around the body part (e.g. limb) of the subject of the therapy.

The actuators 502 (e.g. pistons, servo motors, or others) may exert a force on the moldable membrane 501 directly or indirectly using a mechanism, e.g. lever mechanism or joint mechanism.

At least one actuator 502 may comprise a first base and a second base. The first base may be coupled to the therapy unit 105. The second base of at least one actuator 502 may be connected to the moldable membrane 501. The moldable membrane 501 may form a straight-line profile, an S-shaped profile, an O-shaped profile, a C-shaped profile, or any arbitrary shaped profile. The moldable membrane 501 may have such a profile, which is advantageous in the airtight sealing of the body part.

In one aspect, the actuator 502 may comprise a connecting member 522, which is configured to be coupled to the body part adjacent the moldable membrane 501. The connecting member 522 may be in contact with the body part or in contact with the wrap, if used. The connecting member 522 may be configured to provide a fixation of the body part while the moldable membrane 501 may be configured to provide air sealing of the body part.

The moldable membrane 501 may form a shape with a high contact surface to increase the tightness of the seal. At least one actuator 502 may control one pressure point on the surface of the moldable membrane 501. At least one actuator 502 may control one pressure point, which is part of a number of pressure points that ensure a tight sealing of the subject's body part in the therapy chamber 201. At least one actuator 502 may be in communication with the moldable membrane 501.

The moldable membrane 501 may have a width in a range from 0.001 cm to 20 cm, or from 0.01 cm to 10 cm, or from 0.1 cm to 5 cm, or from 0.5 cm to 3 cm.

The moldable membrane 501 may be made out of a moldable material. The moldable material may have appropriate features to ensure airtight sealing. One of such features may be a tensile strength in a range from 0.01 MPa to 300 MPa, or from 0.1 MPa to 200 MPa, or from 0.1 MPa to 100 MPa, or from 0.1 MPa to 50 MPa, or from 1 MPa to 25 MPa, or from 1 MPa to 10 MPa, or from 4 MPa to 6 MPa.

One such feature may be flexibility (elongation). The moldable material may have flexibility in a range from 1% to 700%, or from 1% to 200%, or from 1% to 100%, or from 200% to 600%, or from 400% to 500%, or from 425% to 475%.

One such feature may be hardness (Shore A). The hardness of the moldable material may be in a range from 0 A to 100 A, or from 5 A to 70 A, or from 5 A to 50 A, or from 10 A to 50 A, or from 15 A to 45 A, or from 20 A to 40 A, or from 25 A to 30 A, or from 33 A to 37 A.

The moldable material may be resistant to high temperatures. The material may have an excellent thermal resistance.

At least one actuator 502 may control at least one pressure point, to ensure tight seal of the subject's body part in the therapy chamber 201.

At least one actuator 502 may control one pressure point, which is part of a number of pressure points that ensure a tight sealing of the subject's body part in the therapy chamber 201. At least two actuators 502 may control one pressure point, which is part of a number of pressure points that ensure a tight sealing of the subject's body part in the therapy chamber 201.

The device may comprise at least one sensor 107, which may measure the force that is applied to the body part by at least one actuator 502. At least one sensor 107, may be in communication with the main unit 101, and may provide feedback about the measured force. The force applied by the actuator may be adjusted based on the feedback from at least one sensor 107. The goal of this aspect may be to provide maximal comfort to the subject during therapy. The feedback ensures a comfortable sealing of the subject's body part in the therapy chamber 201 when a minimal force ensures the airtight sealing is applied to the body part. This aspect may also be beneficial to the efficacy of the vacuum compression therapy (pressure therapy) itself.

The number of actuators 502 may be in the range of 2 to 100, 4 to 50, 8 to 30, 12 to 24, or 14 to 16 actuators. The actuators 502 may be positioned from one side of the moldable membrane 501, or from the other side of the moldable membrane 501, or from both sides of the moldable membrane 501, or inside the moldable membrane 501. In one aspect, some actuators 502 may be positioned from one side of the moldable membrane 501, other actuators 502 may be positioned from the other side of the moldable membrane 501, or from both sides of the moldable membrane 501, or inside the moldable membrane 501.

In one aspect of the device, prior to the therapy, at least one actuator 502 is pulled radially outward, the moldable membrane 501 is pulled radially outward, and the size of the aperture 503 is enlarged. After the body part is inserted into the therapy chamber 201 via the aperture 503 at least one actuator 502 is pulled radially inward, the moldable membrane 501 is pulled radially inward, the size of the aperture 503 is reduced, and an airtight seal between the moldable membrane 501 and the body part is created. After the therapy, the aperture 503 is enlarged again.

At least one actuator 502 may comprise a servomotor. A servomotor may control one pressure point, which is part of a number of pressure points that ensure a tight sealing of the subject's body part in the therapy chamber 201. A pressure point may comprise at least one sensor 107, which provides feedback about the measured force.

At least one actuator 502 may comprise a piston. At least one piston may control one pressure point, which is part of a number of pressure points that ensure a tight sealing of the subject's body part in the therapy chamber 201. At least one piston may be communicating with at least one pressure circuit. At least one pressure circuit may be communicating with the piston pressure generator. The pressure in at least one pressure circuit may be controlled by pressure generator 106 or may be controlled by a separate pressure generator. The pressure force that is applied to the pressure point may be regulated by the pressure in at least one pressure circuit.

A tissue engaging element may be positioned between the moldable membrane 501 and the body part. The tissue engaging surface may provide extra comfort of the seal and may ensure no bruises or marks are formed on the body part. The tissue engaging surface may be made out of any kind of fabric, latex, silicone, hydrogel, saline solution, water-based gels, polymers and others.

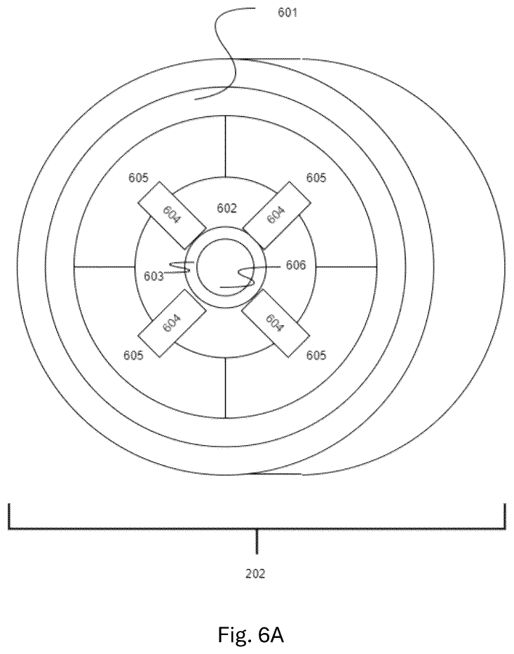

FIG. 6A-B shows additional exemplary schematic diagrams of the sealing unit 202.

In one aspect of the invention, the sealing unit 202 may comprise an iris 601 and a flexible membrane 602. The aperture 606 is formed by the flexible membrane 602 and has an adjustable size.

The iris 601 may ensure a safe and durable fixation of the flexible membrane 602. The iris 601 may also be able to adjust the size of the aperture 606. The iris 601 may comprise at least one blade 605. In one aspect, the iris 601 may comprise a number of blades 605 in a range from 2 to 100, or from 2 to 50, or from 2 to 25, or from 4 to 15, or from 5 to 13.

The flexible membrane 602 may stretch and shrink in order to adjust the size of the aperture 606 due to the elasticity of the flexible membrane 602, where elastic deformation occurs in the flexible membrane 602.

The flexible membrane 602 may be made out of an elastic material. The flexible membrane 602 may be made of latex, silicone, rubber, polyurethane, neoprene, polymer and others. Prior to the therapy, the flexible membrane 602 is in its primary state. The flexible membrane 602 may comprise a sleeve 603. Before the body part is inserted into the therapy chamber 201 through the aperture 606, the sleeve 603 is stretched to its secondary state, and the diameter of the aperture 606 is enlarged. When the body part is in the right position in the therapy chamber 201, the sleeve 603 is shrunk to its sealing state, and the diameter of the aperture 606 is reduced. In the sealing state, the sleeve 603 encloses the body part in the flexible membrane 602.

The flexible membrane 602 may have a width in a range from 0.001 cm to 20 cm, or from 0.001 cm to 10 cm, or from 0.01 cm to 1 cm, or from 0.1 cm to 1 cm.

The flexible membrane 602 may be made out of a flexible material. The flexible material may have appropriate features to ensure airtight sealing. One such feature may be tensile strength in a range from 0.01 MPa to 300 MPa, or from 0.1 MPa to 200 MPa, or from 0.1 MPa to 100 MPa, or from 1 MPa to 100 MPa, or from 1 MPa to 50 MPa, or from 10 MPa to 50 MPa, or from 20 MPa to 30 MPa, or from 22 to 17 MPa.

One such feature may be flexibility (elongation), the flexible material may have a flexibility in a range from 1% to 1500%, or from 1% to 500%, or from 1% to 200%, or from 1% to 100%, or from 50% to 1000%, or from 100% to 1000%, or from 400% to 900% or from 700% to 900%, or from 800% to 850%.

One such feature may be hardness (Shore A), the hardness of the flexible material may be in a range from 0 A to 100 A, or from 5 A to 70 A, or from 5 A to 50 A, or from 10 A to 50 A, or from 15 A to 45 A, or from 25 A to 40 A, or from 30 A to 40 A, or from 35 A to 40 A. The flexible material may be resistant to high temperatures. The material may have excellent thermal resistance.

The flexible membrane 602 may comprise at least one band 604. The at least one band 604 may be made of latex, silicone, rubber, polyurethane, plastics, and other materials. The material of the at least one band 604 may be rigid or flexible. The material of the at least one band 604 may be resistant to tearing and stretching or may be resistant and durable.

The at least one band 604 may improve the strength and durability of the flexible membrane 602 and/or to the sleeve 603. The at least one band 604 may protect the flexible membrane 602 and/or to the sleeve 603 from wearing out and/or tearing.

The number of bands 604 may be in a range from 2 to 100, or from 2 to 50, or from 2 to 25, or from 4 to 15, or from 5 to 10.

The at least one band 604 may have a width in a range from 0.001 cm to 20 cm, or from 0.001 cm to 10 cm, or from 0.01 cm to 1 cm, or from 0.1 cm to 1 cm.

The at least one band 604 may have a length in a range from 0.01 cm to 100 cm, or from 0.1 cm to 50 cm, or from 1 cm to 50 cm, or from 5 cm to 30 cm.

The one or more bands 604 may be positioned on one side of the flexible membrane 602, or on the other side of the flexible membrane 602, or on both sides of the flexible membrane 602, or inside the flexible membrane 602. The device may comprise two irises 601, each positioned on one side of the flexible membrane. Each of the two irises may comprise a number of blades 605.

The at least one band 604 may be connected to the at least one blade 605 of the iris 601. The at least one band 604 may be connected to the sleeve 603. The at least one blade 605 of the iris 601 may communicate with a motor. The motion of the at least one blade 605 that is connected to the at least one band 604 of the iris 601 may enable adjustment of the size of the aperture 606 formed by the sleeve 603. The number of the blades 605 may be the same as the number of the bands 604, or the number of blades 605 may be larger than the number of bands 604. For example, the number of blades 605 may be twice as many as the number of bands 604, or the number of blades 605 may be three, four, or five times higher than the number of bands 604. Or the number of blades 605 may be smaller than the number of bands 604.

The sealing device 202 may be in communication with the at least one sensor 207, that provides feedback about the sealing unit 202, providing maximal comfort to the subject during therapy. The feedback ensures a comfortable sealing of the subject's body part in the therapy chamber 201 when a minimal force ensures the airtight sealing is applied to the body part. This aspect may also be beneficial to the vacuum compression therapy (pressure therapy) itself. This aspect may also reduce the number of times that the seal is not airtight, the number of times that air blows through the seal, applying a force too high to the subject's body part that can be uncomfortable for the subject.

The sealing unit 202 may comprise a combination of the sealing mechanisms. The sealing unit 202 may comprise at least one of the sealing features mentioned above, such as the cuff 401, the inner tubular member 402, the pressure generator of sealing unit, the moldable membrane 501, the least one actuator 502, the iris 601, the flexible membrane 602, the sleeve 603, the at least one band 604, and/or the at least one blade 605.

The sealing unit 202 may be reusable. The sealing unit 202 may be replaceable and/or disposable. Any part of the sealing unit 202, such as, for example, the cuff 401, the moldable membrane 501 and/or the flexible membrane 602 may be reusable and/or replaceable/or disposable.

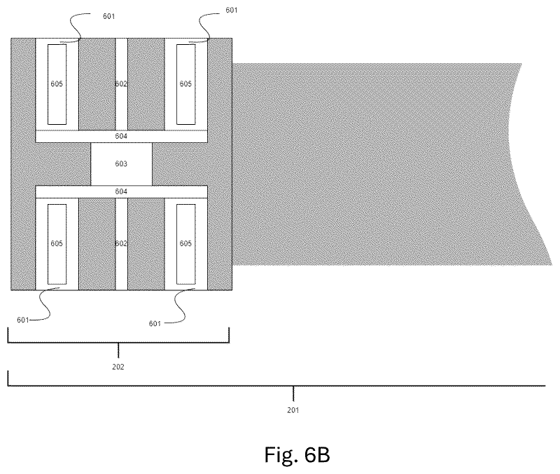

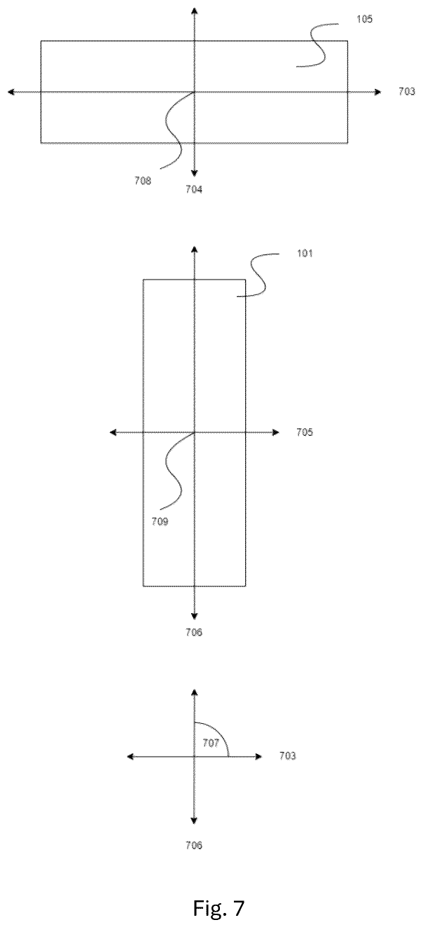

FIG. 7 shows one exemplary schematic diagram of the device. The device may comprise a therapy unit 105. The therapy unit 105 may comprise a first axis 703 and a second axis 704. The first axis 703 of the therapy unit 105 and the second axis 704 of the therapy unit 105 may comprise a center 708.

In one aspect, the therapy unit 105 may comprise a control unit 103, a user interface 104 and at least one sensor 107.

The device may comprise the main unit 101. The main unit 101 may comprise a first axis 705 and a second axis 706. The first axis 705 of the main unit 101 and the second axis 706 of the main unit 101 may comprise a center 709.

In one aspect, the main unit 101 may include the power supply 102, the control unit 103, the user interface 104, the pressure generator 106, and at least one sensor 107.

The device may comprise a positioning mechanism 109. The therapy unit 105 may be connected to the main unit 101 by a positioning mechanism 109. The positioning mechanism 109 may be configured to adjust the position of the therapy unit 105 in relation to the main unit 101.

The main unit may have a height in a range from 10 cm to 200 cm, or from 10 cm to 150 cm, or from 30 cm to 150 cm, or from 50 cm to 150 cm, or from 50 cm to 125 cm, or from 50 cm to 120 cm.

The positioning mechanism 109 may allow various positions of the therapy unit 105 (and its first axis 703) in relation to the main unit 101 (and its second axis 706). The positioning mechanism 109 may allow various lifting and/or tilting, and/or rotating and/or elevating and/or decreasing functions to the therapy unit 105 in relation to the main unit 101. The vertical position, and/or the horizontal position and/or the inclination angle of the therapy unit 105 (and its first axis 703) in relation to the main unit 101 (and its second axis 706) may be adjusted.

The positioning mechanism 109 may comprise at least one of: an actuator, for example an electric motor, axis or holder of the therapy unit or component of the tilt and positioning mechanics, for example threaded rod, joint, lever mechanism, linear guide, or any other appropriate component.

In one aspect of the invention, the positioning mechanism 109 may comprise two actuators formed by electric motors and two threaded rods and the axis or holder of the therapy unit.

The axis or holder of the therapy unit may serve as a channel for the at least one pipe, tubes or cable.

The positioning mechanism 109 may comprise from 1 to 6 degrees of freedom or 2 to 6 or 3 to 6 or 1 to 3 or 2 to 3 degrees of freedom of a position of the therapy unit 105 in relation to the main unit 101. The angle 707 formed by the first axis 703 of the therapy unit 105 and the second axis 706 of the main unit 101 may be in a range from 0° to 180°, or from −90° to 90°, or from 0° to 90°, or from 0° to 120°, or from 0° to 135°, or from 45° to 135° or from 15° to 125°. The therapy unit 105 may be rotated at an overall angle from 0° to 360° and/or from −180° to 180°.

The angle 707 formed by the first axis 703 of the therapy unit 105 and the second axis 706 of the main unit 101 may be adjusted for an angle in a range from 0° to 25°, or from 0° to 20°, or from 0° to 15°, or from 0° to 10°, or from 0° to 5°.

The rotation rate of the therapy unit 105 may be in the range of 0.001°/s to 10°/s, or in the range of 0.001°/s to 9°/s, or in the range of 0.01°/s to 8°/s, or in the range of 0.1°/s to 7°/s, or in the range of 0.5°/s to 6°/s, or in the range of 1°/s to 5°/s. With this system, not only may the therapy unit 105 position be precisely adjusted for the unique needs of a subject and/or therapy, but the system also allows an innovative approach when storing/moving/operating the device. The first axis 703 of the therapy unit 105 and the second axis 706 of the main unit 101 may make an angle of 0° (and/or 180)°. This way, the device takes up very little space, and its storage is very effective.

The device may comprise at least one sensor 107. The sensor 107 may be a sensor of position and/or a sensor of incline. The sensor 107 may provide information about the position of the therapy unit 105 and about the position of the therapy unit 105 in relation to the main unit 101.

Another innovative approach of this positionable mechanism is that it enables consecutive therapy of a first and a second body part, for instance, a first leg and a second leg, and/or a first arm and a second arm, without the need to re-parking the vacuum compression device from a first position to a second position. The operator only has to change the angle 707 formed by the first axis 703 of the therapy unit 105 and the second axis 706 of the main unit 101. This feature may be a great benefit to the operator and/or the subject and/or the therapy, as it lessens the need to manipulate/move the device and prolong the therapy.

The center 708 and center 709 may move relative to each other as the therapy unit 105 moves relative to the main unit 101 for a specific distance. The center 708 and center 709 may comprise a variable distance, the maximum distance may be in a range from 0.001 cm to 200 cm, or from 0.001 cm to 100 cm, or from 0.001 cm to 75 cm, or from 0.001 cm to 50 cm, or from 0.001 cm to 25 cm, or from 0.001 cm to 10 cm, or from 0.001 cm to 0.01 cm, or from 1 cm to 100 cm, or from 10 cm to 75 cm, or from 20 cm to 50 cm.

The centers 708 and 709 may change their relative position in a horizontal direction, vertical direction, diagonal direction, backward and forward direction, or in a combination of all.

In one aspect of the invention, the main unit 101 may comprise a front-mounted user interface 104, meaning that the user interface 104 is positioned in front of the therapy unit 105 from the perspective of the user or therapy provider, as depicted in FIG. 9. This configuration may enable movement and/or rotation of the therapy unit 105, that may be formed for example by the therapy chamber, between at least a portion of the main unit 101 and the user interface 104. The therapy unit 105 may comprise the sealing unit 202. The therapy unit 105 may be placed between the stand 150 of the main unit and the user interface 104, wherein the stand 150 of the main unit is the portion of the main unit 101. Such an arrangement is beneficial for the user or therapy provider, for example, for more comfortable control of the device. In this aspect, the main unit 101 may comprise an interface holder 111 allowing the placement of the user interface 104 in front of the therapy unit 105 as seen by the user or therapy provider. The interface holder 111 may be connected to both the user interface 104 and the main unit 101. The interface holder 111 may be connected to the stand 150 of the main unit 101. The user interface 104 may be placed in a vertical position, or may be tilted at an angle relative to the vertical position. The user interface 104 may tilted in a direction towards the user or therapy provider.

The device according to the invention may comprise a front-mounted user interface 104, where the therapy unit 105 is disposed between at least a portion of the main unit 101 and the user interface 104.

The angle between the user interface 104 and the vertical plane may be in the range of 0 to 90°, or in the range of 0 to 60°, or in the range of 0 to 45°, or in the range of 0 to 30°, or in the range of 0 to 25°, or in the range of 0 to 20°, or in the range of 0 to 15°, or in the range of 0 to 10°, or in the range of 0 to 5°.

The interface holder 111 may have a shape of a plane, cylinder, rod, sphere, cuboid, prism, or a modification thereof, or may have any other appropriate shape. The interface holder 111 may be placed in a plane perpendicular to the plane of the user interface 104, or may be tilted at an angle relative to the plane perpendicular to the plane of the user interface 104. The angle between the plane of interface holder 111 and the plane perpendicular to the plane of the user interface 104 may be in the range of 0 to 90°, or in the range of 0 to 60°, or in the range of 0 to 45°, or in the range of 0 to 30°, or in the range of 0 to 25°, or in the range of 0 to 20°, or in the range of 0 to 15°, or in the range of 0 to 10°, or in the range of 0 to 5°.

The bottom part of the device may comprise a base 115.

In one aspect of the invention, the device may comprise two parts, an upper part and a bottom part, as depicted in FIG. 10. The virtual line 551 separating the two parts of the device may be placed horizontally perpendicular to the stand 150 (not depicted in FIG. 10) approximately in the first third of the height of the device from the ground.

The upper part of the device may comprise the user interface 104, the positioning mechanism 109 and the therapy unit 105. The upper part of the device may further comprise (not depicted in FIG. 10) at least one of: the interface holder 111, electronics, a printed circuit board, a processor, cable, tube, filter, e.g. air filter, or any component for service and cleaning of the device, or any other component.

In another aspect, the virtual line 551 separating the two parts of the device may be placed horizontally perpendicular to the stand 150 approximately at first quarter or at half of the height of the device from the ground, or the virtual line 551 may be placed horizontally perpendicular to the stand 150 at a height of the device between the base 115 and the stand 150.

The bottom part of the device may comprise parking system 110 and the pressure generator 106. The bottom part of the device may further comprise (not depicted in FIG. 10) at least one of: electronics, printed circuit board, a processor, a power supply, cable, tube or valve, or any other component.

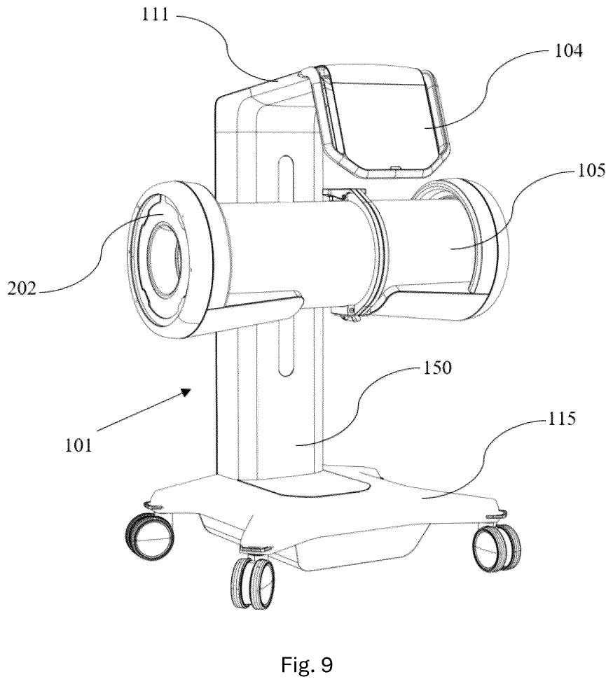

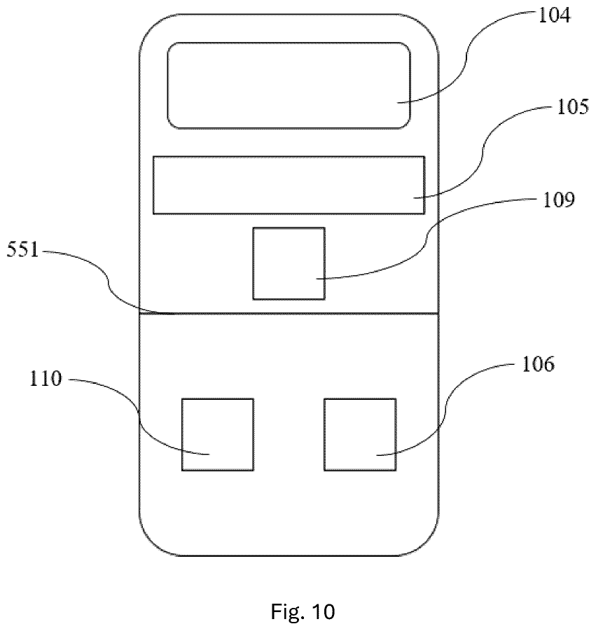

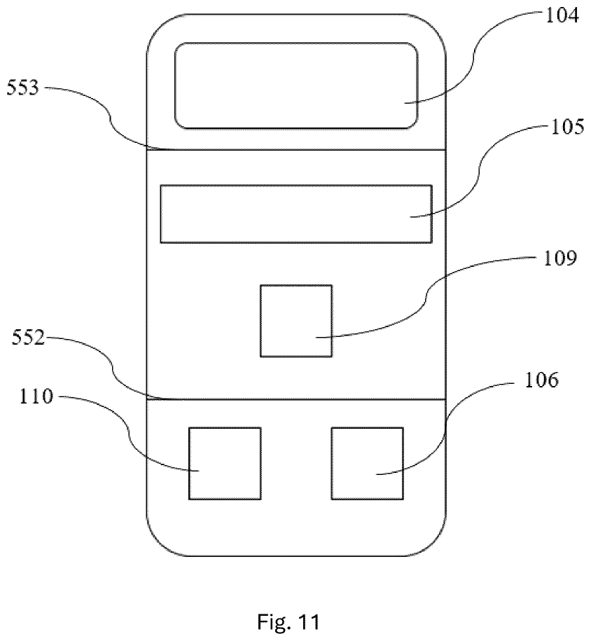

In another aspect of the invention, the device may comprise three parts, an upper part, a middle part and a bottom part, as depicted in FIG. 11. The first virtual line 552 separating the first and second part of the device may be placed horizontally perpendicular to the stand 150 (not depicted in FIG. 11) approximately in the first third of the height of the device from the ground. The second virtual line 553 separating the second and third part of the device may be placed horizontally perpendicular to the stand 150 (not depicted in FIG. 11) approximately in the second third of the height of the device from the ground.

In other aspect, the first virtual line 552 separating the first and second parts of the device may be placed horizontally perpendicular to the stand 150 (not depicted in FIG. 11) at the height of the device, between the base 115 and the stand 150. The second virtual line 553 separating the second and third parts of the device may be placed horizontally perpendicular to the stand 150 (not depicted in FIG. 11) approximately at the height of the device between the therapy unit 105 and the user interface 104.

The upper part of the device may comprise the user interface 104. The upper part of the device may further comprise (not depicted in FIG. 11) at least one of: the interface holder 111, electronics, a printed circuit board or processor, or any other appropriate component.

The middle part of the device may comprise the positioning mechanism 109 and the therapy unit 105. The middle part of the device may further comprise (not depicted in FIG. 11) at least one of: a cable, tube, filter, e.g. air filter, or any component for service and cleaning of the device, or any other component.

The bottom part of the device may comprise a parking system 110 and the pressure generator 106. The bottom part of the device may further comprise (not depicted in FIG. 11) at least one of: electronics, a printed circuit board, a processor, power supply, cable, tube or valve, or any other component.

The device according to the invention may comprise a moving arm or any other appropriate feature, that may position and manipulate therapy unit 105 within a 3D workspace. The moving arm or any other appropriate feature may be capable of precise movement of the therapy unit 105 in maximally three linear axes. The moving arm or any other appropriate feature may move the therapy unit 105 in at least one, or in at least two, or in three axes. The axes may comprise an X-axis for horizontal movement (left to right or side to side), a Y-axis for vertical movement (up and down) and a Z-axis for depth movement (forward and backward). The moving arm or any other appropriate feature may allow rotation of the therapy unit 105 around any of the aforementioned axes.

The moving arm or any other appropriate feature may be connected to the main unit.

The moving arm or any other appropriate feature may comprise at least one of: a linear actuator or a guide, that may translate rotational motion, e.g. from motor into linear motion. The linear actuator may comprise a stepper or servo motor, lead screw, ball screw, belt drive, linear rail or slider, arm or end-effector holder or a control system, or any other appropriate component.

The arm or end-effector holder may be mounted on a moving platform, it may carry a component or tool head. The arm or end-effector holder may move in all three directions to reach any point within the working space.

The control system may control movements along each axis using a controller (e.g. microcontroller or industrial controller) and/or translate commands into precise motor actions.

The moving arm or any other appropriate feature for positioning the therapy unit 105 may comprise a mechanical device without any electric or other actuator, e.g. electric motor.

The main unit 101 may comprise a bottom plate. The bottom plate may be equipped with a parking system 110. The parking system 110 may comprise at least one wheel, and/or at least two wheels, and/or at least four wheels. The parking system 110 may be adjusted to allow an easy, safe, and comfortable movement and/or positioning and/or parking of the device around the operating room.

The parking system 110 may comprise a central brake 114 that may be in communication with the control unit 103, the therapy unit 105, at least one sensor 107, the power supply 102, the user interface 104, and the pressure generator 106. The at least one sensor 107 may be an accelerometer. The at least one sensor 107, may provide feedback about the state of the device. In the case of a safety hazard to the subject, the central brake 114 may be automatically locked and/or unlocked to ensure the safety of the subject. The central brake 114 may be in communication with the user interface 104, and the central brake 114 may be locked/unlocked using the user interface 104. The device may also comprise a mechanical brake.

The central brake 114 may be locked and/or unlocked automatically, or manually. The central brake 114 may be locked and/or unlocked by pressing a button and/or by touching the user interface, for example the control panel with display.

The central brake 114 may be automatically locked and/or unlocked according to the status of the therapy, for example at the time of start or end of the therapy, or at another appropriate time during the therapy, e.g. during a pause in the therapy. This setting is time saving and comfortable for the operator of the therapy.

The central brake 114 may lock at least one wheel of the parking system, and/or at least two wheels, and/or at least four wheels, or all wheels of the parking system.

The device may comprise a safety valve. The safety valve may be in communication with the pressure generator 106. The safety valve may be in communication with the control unit 103. The safety valve may be used when the pressure in the therapy chamber 201 exceeds a safe pressure limit. When the safe pressure limit is exceeded, the valve is opened and the pressure in the therapy chamber 201 is equalized to the atmospheric pressure. The safe pressure limit may be in a range from 20 kPa to 100 kPa, or from 20 kPa to 75 kPa, or from 20 kPa to 50 kPa, or from 20 kPa to 40 kPa, or from 24 kPa to 50 kPa, or from 24 kPa to 30 kPa.

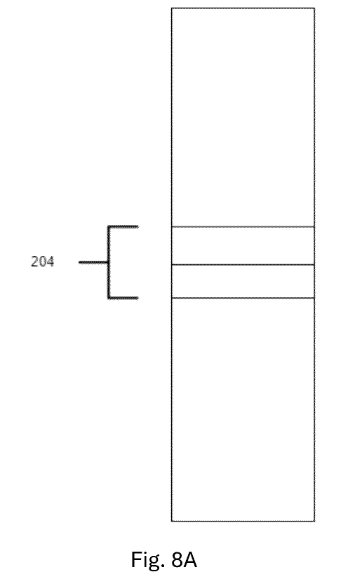

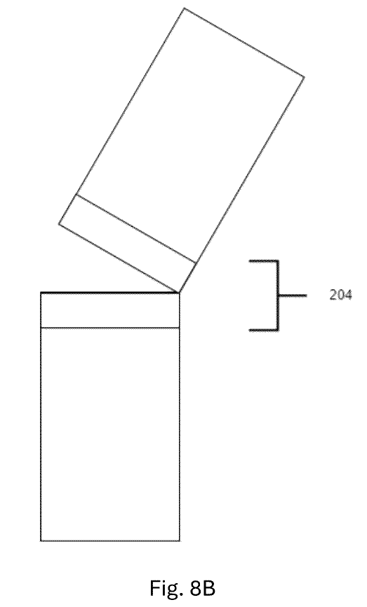

FIGS. 8A-B show an exemplary diagram of the therapy chamber 201 comprising at least one opening 204. The FIG. 8A shows the exemplary diagram of the therapy chamber 201 when the at least one opening 204 is in a closed state. The FIG. 8B shows the exemplary diagram of the therapy chamber 201 when the at least one opening 204 is in an open state.

The therapy chamber 201 may be made out of two parts.

The therapy chamber 201 may comprise at least one opening 204. The at least one opening 204 may be located between the two parts of the therapy chamber 201. The at least one opening 204 may enable easier access to the therapy chamber 201, may enable an easier cleaning and disinfecting of the therapy chamber 201, and may enable a port to add any additional treatment elements 108 to the therapy chamber 201. The at least one opening 204 may be configured to create an airtight seal in the therapy chamber 201. The seal may be a rubber seal, a silicone seal, an inflatable seal, a zipper seal, an O-ring seal, or any other appropriate seal.

In one aspect of the invention, therapy chamber 201 may comprise at least two parts connected together and at least one opening 204. The at least one opening 204 may be located between the two parts of the therapy chamber 201.

The therapy chamber 201 may be opened and closed in at least one opening 204. The at least one opening 204 may comprise a closing mechanism for opening and closing the therapy chamber 201. The closing mechanism may comprise at least one of: a lid, a cap, a closure or any other appropriate component for closing the therapy chamber 201. The closing mechanism may further comprise at least one of: a latch, screws, bayonet mechanism or click mechanism, or any other appropriate mechanism.

The closing mechanism may comprise at least one seal.

The closing mechanism may comprise a spring or any other appropriate component that slows down or brakes the movement of opening and closing the therapy chamber 201. In another aspect of the invention, the therapy chamber 201 may comprise at least one opening 204. The at least one opening 204 may comprise at least one seal. The at least one opening 204 may be sealed using an inflatable seal. The inflatable seal may be in communication with an inflatable seal pressure generator. The inflatable seal pressure generator may be the pressure generator 106.

The therapy chamber 201 may comprise at least one opening 204. The at least one opening 204 may be located at the first base 207 of the therapy chamber 201, or at the second base 208 of the therapy chamber 201. The at least one opening 204 may be located on the base where the sealing unit 202 is not located.

The at least one opening 204 may comprise closing mechanism for opening and closing the therapy chamber 201. The closing mechanism may be located on the side of the therapy chamber 201 where the sealing unit 202 is not located. The closing mechanism may comprise at least one of: a lid, a cap, a closure or any other appropriate component for closing the therapy chamber 201. The closing mechanism may further comprise at least one of: a latch, screws, bayonet mechanism or click mechanism, or any other appropriate mechanism.

The therapy chamber 201 may comprise at least two openings 204. The first one of the at least two openings 204 may be located between the two parts of the therapy chamber 201. The second one of the two openings may be located on the base where the sealing unit 202 is not located.

The device may comprise an additional system for positioning the subject. The system for positioning the subject may be a sitting surface (such as a chair) and/or a lying surface (such as a couch and/or a bed). The system for subject positioning may be a part of the device or may be a separate system. The sitting surface and/or the lying surface may be adjusted to provide the best results in combination with the device.

A sitting and/or lying surface may comprise a first part and a second part. In one aspect, the first and the second part of the sitting and/or lying surface may be located outside the therapy unit.

In another aspect, the first part of the sitting and/or lying surface may be located outside the therapy unit, and the second part of the sitting and/or lying surface may be located inside the therapy unit.

The sitting and/or lying surface may comprise a plurality of at least two parts located inside the therapy unit and/or a plurality of at least two parts located outside the therapy unit.

The sitting surface, such as a chair, may comprise a seat and a backrest. The seat of the chair may be adjusted to fit well with the vacuum compression therapy (pressure therapy) chamber 201. The seat may have a length in a range from 10 cm to 200 cm, or from 10 to 100 cm, or from 25 cm to 75 cm. The seat may have a width in a range from 10 cm to 200 cm, or from 10 to 100 cm, or from 25 cm to 75 cm. The length and/or the width of the seat may be variable and may be adjusted.

The lying surface, such as the bed, may have a length in a range from 10 cm to 300 cm, or from 100 cm to 300 cm, or from 150 cm to 250 cm, or from 180 cm to 220 cm. The lying surface may have a width in a range from 10 cm to 200 cm, or from 10 to 100 cm, or from 25 cm to 75 cm. The length and/or the width of the lying surface may be variable and may be adjusted.

The device may comprise a belt and/or a harness to ensure that the subject's body part is safely secured and/or have an airtight seal in the therapy unit 105. The belt and/or harness may be in contact with the subject's body surface and may be made out of a rigid or at least partially flexible material. The belt may comprise a fastening mechanism, such as, for example, snap, clamp, rails, zipper, holes, hook and loop system (e.g. Velcro), and others. The belt and/or harness may be in communication with the main unit 101, a power supply 102, a control unit 103, a user interface 104, therapy unit 105, pressure generator 106, and/or at least one sensor 107.

The device may comprise a bag or an overcoat to seal the subject's body part, or a part of the subject's body part to ensure no contamination and sterility of the therapy unit 105 and/or the therapy chamber 201. The bag or an overcoat may be used when there is a wound present on the body part.

The bag or overcoat may be made out of rubber, silicone, latex, polymer or plastic, and others.

The device may also comprise a sheet or a cloth to cover the body part, or a part of the subject's body part. The sheet may be releasably adhered to the surface of the subject's body part. The sheet or cloth may be used to withdraw the fluid from the wound during the vacuum compression therapy (pressure therapy). Various other therapeutic means may be inserted into the therapy unit 105 and/or the therapy chamber 201 to provide effective therapy.

The device may also comprise a means to disinfect the therapy unit 105 and/or the therapy chamber 201. The disinfecting means may be, for example, a pipe and/or a nozzle, that contains a fluid disinfectant that is sprayed into the therapy unit 105 and/or the therapy chamber 201. The disinfecting means may also be in the form of radiation, for example, UV-radiation that has disinfecting and germicidal effects.

The device may include a user interface 104. The user interface 104 may be included in the main unit 101 and/or in the therapy unit 105. The user interface 104 may include at least one of several buttons, a stop button, LCD display, keyboard, switch, touchpad, and/or a circular control element. The user interface 104 may show various parameters that are relevant to the ongoing therapy.

The parameters may be at least one of the pressure in the therapy chamber 201, the flow of air into the therapy chamber, the pattern of the therapy, the duration of one of the therapy periods, the pressure changes between two consecutive therapy periods, the temperature in the therapy chamber 201, the position of the therapy unit 105 and others, information about the subject and/or error messages.

The operator may set various therapy parameters and various therapy procedures through the user interface 104. The therapy parameters may include at least the pressure in the therapy chamber 201, the flow of air into the therapy chamber 201, the pattern of the therapy, the duration of one of the therapy periods, the pressure changes between two consecutive therapy periods, the temperature in the therapy chamber 201, the position of the therapy unit 105 and others. Additionally, the operator may set various parameters of at least one treatment element 108.

Therapy parameters may be changed before the therapy or during the therapy. The software may include one or more predefined protocols, from which the operator may choose the most suitable one. In one aspect, the one or more predefined protocols may be selected by the operator of the device via the user interface 104. The selected protocol then may be executed by the control unit 103. In another aspect, the operator may also create his own protocol, for example via the user interface 104.

The device may include a database of subjects, providing information about the subjects and providing information about previous/ongoing therapies. The device may include a counter, which can count the number of performed therapies, the time of providing specific therapies, used therapy parameters, and others. Data from the counter may be stored in the device's memory or in an external storage. The device may be able to download new therapy protocols and/or new software.

Methods of therapy may include preparation of the subject, sealing of the device, active operation of the device, discontinuation, and ceasing operation. These phases of the method may follow the disclosed order or their order may be changed. Also, there may be more than one of the same phases used during a method of therapy. Such situations may occur for example when the active operation is interrupted by a discontinuation and later the active operation continues. Also, the method of therapy may not include all phases of therapy, e.g. when the operation is ceased immediately after active operation. The pressure of the gas in the chamber may be changed during all steps of the method.

Preparation of the subject may include positioning the subject on the subject support and/or sealing the subject into the therapy chamber 201. The subject and/or part of the subject's body may then be moved inside the therapy chamber 201 such that the therapy chamber 201 surrounds part or all of a body part e.g. arm, hand, leg, foot, torso. Alternatively, the therapy chamber 201 may be moved on the subject's body, while the subject support may not move.

Sealing of the subject into the therapy chamber 201 may include operating the sealing unit 202 and adjusting the sealing unit 202 into a position that comfortably seals the body part. The device may be calibrated. Calibration of the device may include the creation of an initial pressure gradient testing the air-tightness of the therapy chamber 201 and/or the sealing unit 202. Also, the calibration of the device may include a collection of initial data from the at least one sensor 107.

Active operation may include generating pressure with at least one pressure generator 106 and/or application of at least one type of additional type of energy by the treatment element 108 to the tissue.

The application of the additional type of energy by the treatment element 108 may precede or follow or be simultaneous to the application of the pressure. The application of the additional type of energy may overlap with the application of the pressure. The overlap of the additional type of energy with the pressure may occur at discrete time intervals in the range of 0.01 s to 1200 s.

During active operation, the at least one sensor 107 may provide information about the speed and/or amount of gas flowing into and/or out of the therapy chamber 201, the temperature of the gas, the concentration of at least one gas, ionization of the gas and/or humidity of the gas. The at least one sensor 107 may be a flow sensor, temperature sensor, concentration sensor, chemical sensor and/or photonic sensor. Information provided by the gas monitoring sensor may be used in conjunction with data related to the tissue. Such data may be used for repeated calibration of the device, determination of the optimal amount of gas present in the chamber, determination of sufficient flow of the gas into the chamber and determination of sudden unwanted pressure changes related to movement of the subject and/or leakage.

Discontinuation may be an effect of the operator's and/or the subject's wish to temporarily cease active operation. Discontinuation may be initiated by the operator's command, the subject's command and/or a safety command, wherein the command may be given through the user interface 104 and/or control unit 103. When the control unit 103 determines that the subject may be in danger, the active operation is stopped. During discontinuation, the operator, control unit 103 and/or subject may change the parameters related to the pressure gradient and/or additional type of energy.

Ceasing operation may include stopping therapy and an additional type of energy and/or balancing the pressure inside the therapy chamber 201 in relation to the outside environment.

Methods of therapy may include feedback. A control unit 103 may control the reaction of the device during all parts of therapy. Feedback may include a reaction to a change in at least one of the previously mentioned parameters, detected by at least one sensor 103 and/or calculated by the control unit 103.

Feedback may include a reaction to a change in at least one parameter related to tissue. When the temperature of the tissue is increased and/or decreased by about 0.5° C. from a set temperature, for example, the device may increase and/or decrease the flow speed in the range of about 0.1 cm/s to 2 m/s. In another aspect, the device may increase and/or decrease the moisture level of the fluid. In still another aspect, the device may increase and/or decrease the temperature of the fluid in the range of 0.1° C. to 10° C. from the actual temperature. In still another aspect the device may balance pressure in the therapy chamber 201 with the atmospheric pressure. In still another aspect the device may cease the application of an additional type of energy and/or change its parameters e.g. frequency, power, pulse duration, focal spot size, location of focus, current, voltage and/or duration of application.

When the volume of the tissue is increased and/or decreased from about 1% to 10% of the volume before therapy, the device may increase and/or decrease the flow speed in the range of about 0.1 cm/s to 2 m/s. In still another aspect the device may balance pressure in the chamber with the atmospheric pressure. In still another aspect, the device may cease the application of additional types of energy and/or change its parameters e.g. frequency, power, pulse duration, focal spot size, location of focus, current, voltage and/or duration of application.

When the moisture level of the tissue is increased and/or decreased from about 5% of the moisture level before therapy, the device may increase and/or decrease the flow speed in the range of about 0.1 cm/s to 2 m/s. In still another aspect, the device may balance the pressure in the chamber with the atmospheric pressure. In another aspect, the device may increase and/or decrease the moisture level of the fluid. In still another aspect, the device may cease the application of additional types of energy and/or change its parameters e.g. frequency, power, pulse duration, focal spot size, location of focus, current, voltage and/or duration of application.

When the pH of the tissue is increased and/or decreased from about 0.5 pH unit from the pH level before therapy, the device may increase and/or decrease the flow speed in the range of about 0.1 cm/s to 2 m/s. In another aspect, the device may increase and/or decrease the moisture level of the fluid. In still another aspect, the device may cease the application of additional types of energy and/or change its parameters e.g. frequency, power, pulse duration, focal spot size, location of focus, current, voltage and/or duration of application.

When the blood saturation of the tissue is decreased and/or increased from about 1% unit of the blood saturation before therapy, the device may increase and/or decrease the flow speed in the range of about 0.1 cm/s to 2 m/s. In another aspect, the device may balance the pressure in the chamber with the atmospheric pressure. In still another aspect the device may increase and/or decrease the moisture level of the fluid. In still another aspect the device may cease the application of additional types of energy and/or change its parameters e.g. frequency, power, pulse duration, focal spot size, location of focus, current, voltage and/or duration of application.

When the heart rate, systolic pressure, and/or diastolic pressure of the subject is decreased by about 1% from the level before therapy the device may increase and/or decrease the flow speed in the range of about 0.1 cm/s to 2 m/s. In another aspect, the device may balance the pressure in therapy chamber 201 with the atmospheric pressure.

When the systolic and/or diastolic blood pressure of the tissue is decreased from about 1% in relation to the blood pressure before therapy, the device may increase and/or decrease the flow speed in the range of about 0.1 cm/s to 2 m/s. In another aspect, the device may balance pressure in the therapy chamber 201 with the atmospheric pressure. In still another aspect the device may increase and/or decrease the moisture level of the fluid. In still another aspect, the device may cease application of additional types of energy and/or change its parameters e.g. frequency, power, pulse duration, focal spot size, location of focus, current, voltage and/or duration of application.

When the muscle activity is changed (e.g. one or more muscles do not contract and/or relax), the device may increase and/or decrease the flow speed in the range of about 0.1 cm/s to 2 m/s. In another aspect, the device may balance pressure in chamber with the atmospheric pressure. In still another aspect the current, voltage, duration and/or frequency of electric energy may be changed. In still another aspect the intensity, flux density, repetition rate, duty cycle and/or impulse duration of the magnetic field may be changed. In still another aspect the frequency, wavelength, power and/or focal spot size may be changed.

All aspects above may be combined. The foregoing description of preferred aspects has been presented for purposes of illustration and description. It is not intended to be exhaustive or to limit the invention to the precise form disclosed. Modification and variations are possible in light of the above teachings or may be acquired from practice of the invention and its practical application to enable one skilled in the art to utilize the invention. Various modifications as are suited to a particular use are contemplated. It is intended that the scope of the invention be defined by the claims appended hereto and their equivalents.

In one aspect of the invention, a device for vacuum compression therapy (pressure therapy), configured to treat subject's body, may comprise a main unit, a therapy unit, a pressure generator and a positioning mechanism 109. The therapy unit 105 may comprise a therapy chamber 201, wherein the therapy chamber 201 may be configured to enclose and seal a part of the subject's body. The pressure generator may be configured to introduce periods of various pressure in the therapy chamber 201. The positioning mechanism 109 may be configured to adjust the position of the therapy unit in relation to the main unit in 1 to 6 degrees of freedom. The pressure generator may be configured to introduce periods of positive and negative pressure in the therapy chamber 201.

In another aspect, the positioning mechanism 109 may be configured to adjust the position of the therapy unit in relation to the main unit in 2 to 3 degrees of freedom.

In another aspect, the pressure generator described may comprise a compressor.

In another aspect, the positioning mechanism 109 may be configured to provide lifting and/or tilting, and/or rotating and/or elevating and/or decreasing functions to the therapy unit in relation to the main unit.

In another aspect, the therapy unit may comprise a first axis and the main unit may comprise a second axis, wherein the positioning mechanism 109 may be configured to tilt the therapy unit in relation to the main unit, wherein the angle formed by the first axis of the therapy unit and the second axis of the main unit by tilting is in a range from 0° to 180°.

In another aspect, the part of subjects' body may be sealed in the therapy chamber 201 by a sealing unit 202, wherein the sealing unit 202 comprises at least one cuff.

In another aspect, the pressure difference between the pressure in the therapy chamber 201 and the atmospheric pressure is in a range from 0.001 kPa to 1000 kPa.

In another aspect, the pressure changes introduced by the pressure generator may be in a range from 0.001 kPa/second to 1000 kPa/second.

In one aspect of the invention, the pressure in the sealing unit 202 may be dynamically changed based on the pressure in the therapy unit 105, for example formed by the therapy chamber 201.

The pressure in the sealing unit 202 may be adjusted based on a target pressure in the therapy unit 105 or based on the positive pressure threshold and/or the negative pressure threshold.

The target pressure in the therapy unit 105 is requested pressure, that is appropriate for the therapy.

For example, during transitions of pressure in the therapy unit 105 from positive to negative pressure, the pressure in the sealing unit 202 may be reduced. It is more pleasant for the patient subjected to the therapy, because blood flow may be relieved for a while during this phase.

The pressure in the sealing unit 202 may be reduced for a time period in the range of 0.001 seconds to 10000 seconds, or in the range of 0.1 seconds to 5000 seconds, or in the range of 1 second to 1000 seconds, or in the range of 1 second to 500 seconds, or in the range of 120 seconds to 1200 seconds, or in the range of 120 seconds to 1000 seconds, or in the range of 60 seconds to 600 seconds, or in the range of 60 seconds to 360 seconds, or in the range of 1 second to 120 seconds, or in the range of 1 second to 50 seconds, or in the range of 1 second to 20 seconds, or in the range of 5 seconds to 15 seconds, or in the range of 1 seconds to 10 seconds, or in the range of 1 second to 5 seconds, or in the range of 2 seconds to 3 seconds.

The pressure in the sealing unit 202 may be reduced during the atmospheric pressure phase in the therapy unit 105 and/or in phases when the pressure in the therapy unit 105 is higher or lower than atmospheric pressure. The pressure in the sealing unit 202 may be dynamically changed based on the pressure in the therapy unit 105, wherein the pressure may be reduced from the pressure higher than atmospheric down to atmospheric pressure.

The pressure in the sealing unit 202 may be changed based on the pressure in the therapy unit 105 in such a way that pressure leaks in the therapy unit 105 are monitored by the pressure sensor and the pressure in the sealing unit 202 may be reduced or increased depending on the pressure leaks in the therapy unit 105. The pressure in the sealing unit 202 may be increased when the pressure leaks are detected in the therapy unit.

In further aspect of the invention, a device for vacuum compression therapy (pressure therapy), configured to treat subjects' body, may comprise a therapy unit and a pressure generator. The therapy unit may comprise a therapy chamber 201, wherein the therapy chamber 201 may be configured to enclose and seal a part of subject's body and wherein the therapy chamber 201 may comprise at least one opening configured to enable easier access to the therapy chamber 201. The pressure generator may be configured to introduce periods of various pressure in the therapy chamber 201.

In one aspect, the device may comprise at least one sensor for measuring blood saturation.

In another aspect, the device may comprise a treatment element that provides at least one additional type of energy.

Furthermore, the pressure difference between the pressure in the therapy chamber 201 and the atmospheric pressure may be in a range from 0.001 kPa to 1000 kPa.

In one aspect, the therapy chamber 201 may comprise a first axis, wherein the at least one opening is centralized with the first axis of the therapy chamber 201.

In another aspect, the therapy chamber 201 may comprise a first base and/or a second base and the at least one opening may be distanced from the first base and/or the second base for a distance in a range from 1 cm to 75 cm.

In yet another aspect of the device, a device for vacuum compression therapy (pressure therapy), configured to treat subjects' body, may comprise a therapy chamber 201, a pressure generator and a sealing unit. The therapy chamber 201 may be configured to enclose a part of the subject's body. The pressure generator may be configured to introduce periods of various pressure in the therapy chamber 201. The sealing unit may be configured to airtightly seal the part of the subject's body in the therapy chamber 201, wherein the sealing unit may comprise an adjustable aperture with an adjustable diameter that can be adjusted to airtightly seal parts of the subject's body parts of various diameter sizes, and wherein the sealing unit comprises a moldable membrane and at least one actuator.

Furthermore, the moldable membrane may comprise a moldable material, wherein the moldable material may have a tensile strength in a range from 0.01 MPa to 300 MPa.

The moldable membrane may use for sealing the patient's body part, e.g limb under a pressure that may be in the range of 0 to 1000 kPa, or in the range of 0.001 to 900 kPa, or in the range of 0.1 to 800, or in the range of 1 to 700 kPa, or in the range of 10 to 600 kPa, or in the range of 100 to 500 kPa, or in the range of 150 to 400 kPa. The pressure may be measured in the moldable membrane mechanics, for example the pressure in the hydraulic system or the pneumatic system of the moldable membrane may be measured.

The pressure force of the sealing unit 202, comprising for example moldable membrane, cuff, or any other sealing, to the patient's body part, e.g. limb, may be in the range of 0 to 10 N/cm², or in the range of 0.001 to 9 N/cm², or in the range of 0.01 to 8 N/cm², or in the range of 0.1 to 7.5 N/cm².

In another aspect, the moldable membrane may comprise a moldable material, wherein the moldable material may have a flexibility in a range from 1% to 700%.

In one aspect, the adjustable aperture may have a diameter in a range from 1 cm to 100 cm.

In another aspect, the diameter of the adjustable aperture may vary during the therapy.

Additionally, the at least one actuator may be a piston.

The device for pressure therapy, the device may comprise a control unit 103, a therapy unit 105, and a pressure generator 106.

The therapy unit 105 may comprise the therapy chamber 201.

The device for pressure therapy may further comprise a sealing unit 202.

The sealing unit 202 may comprise at least one of: a cuff 401, a moldable membrane 501, a flexible membrane 602, an iris 601, an inner tubular member 402, a pressure generator 404, an actuator 502, a sleeve 603, a band 604, a blade 605 or a wrap, or a combination thereof.

The cuff 401 may be inflatable.

The device may comprise a cuff release barrier for attachment of the cuff 401 in the therapy chamber 201 and prevent it from being released or falling out of the therapy chamber 201.

The sealing unit 202 comprising a click mechanism 113.

The click mechanism 113 may comprise:
an inserting member 117 for inserting the cuff 401;
a cuff connector 116 for air tight connection of the cuff 401, wherein the cuff connector 116 is connected to the pressure generator 106 or to the pressure sensor; and
a closing member for closing the cuff 401 into the inserting member 117.

The sealing unit 202 may comprise at least one actuator 502, wherein the actuator 502 is hydraulic, pneumatic or electric, and the actuator 502 comprises at least one of a piston, a motor, a servo motor, a liner actuator or rotary actuator, or a combination thereof.

The device may further comprise a main unit 101, wherein the main unit 101 may be connected to therapy unit 105 by at least one fluid conduit 203; the device further comprising a user interface 104.

The device may further comprisie a pressure generator 106 connected to the therapy unit 105 by at least one fluid conduit 203, the device may further comprise at least one sensor 107.

The device may comprise a positioning mechanism 109 to adjust the position of the therapy unit 105.

The device may comprise a parking system 110 for positioning or parking of the device, the parking system 110 may comprise at least one wheel and a central brake 114.

The device may comprise two parts, wherein the upper part may comprise the user interface 104, the positioning mechanism 109 and the therapy unit 105, the bottom may comprise the parking system 110 and the pressure generator 106.

The device may comprise three parts, wherein the upper part may comprise the user interface 104, the middle part may comprise the positioning mechanism 109 and the therapy unit 105, the bottom part may comprise the parking system 110 and the pressure generator 106.

The device may comprise a front-mounted user interface 104, wherein the therapy unit 105 may be disposed between the at least portion of the main unit 101 and the user interface 104.

The device for pressure therapy may comprise:
a control unit 103;
a pressure generator 106 configured to generate positive and negative pressure;
a therapy unit 105 comprising a sealing unit 202, wherein the sealing unit 202 comprising at least one of: a cuff 401, a moldable membrane 501, a folded cuff 512 or a an iris 601 and a flexible membrane 602.

The therapy unit 105 may comprise the therapy chamber 201 having a cylindrical cross-section.

The therapy unit 105 may comprise therapy chamber 201 having at least one opening 204 allowing access to the therapy chamber 201, the opening 204 may be located on the side of therapy chamber 201, where the sealing unit 202 is not located.

The opening 204 may comprise at least one seal.

The sealing unit 202 may comprise the cuff 401 coupled to the therapy chamber 201 using at least one cuff release barrier.

The sealing unit 202 may comprise a moldable membrane 501, at least one actuator 502 and an aperture 503.

The aperture 503 may have an adjustable size.

The at least one actuator 502 may be hydraulic, pneumatic, or electric.

The at least one actuator 502 may comprise at least one of: a piston, a motor, a servo motor, a linear actuator or rotary actuator.

The at least two actuators may be arranged in a circular manner.

The moldable membrane 501 may have at least one profile of: a straight-line profile, an S-shaped profile, an O-shaped profile or C-shaped profile.

The moldable membrane 501 may comprise a facet 506 and a body 511, wherein a width of the facet 506 is greater than a width of the body 511 of the moldable membrane, the width is determined in cross section through the membrane.

The moldable membrane 501 may be made from at least one of: polymer material, silicone, latex, natural latex, organic latex, synthetic latex, blended latex, neoprene, rubber or polyurethane.

The sealing unit 202 may comprise a folded cuff 512.

The sealing unit 202 may comprise two folded cuffs 512 allowing airtight sealing of the body part of the subject of the therapy when both negative and positive pressures relative to the atmospheric pressure are used during the therapy.

The device may comprise a thermal treatment element.

The device for pressure therapy may comprise:
a control unit 103, a therapy unit 105, and a pressure generator 106,
wherein the therapy unit 105 may comprise the therapy chamber 201 having at least two parts, wherein the therapy chamber 201 may comprising at least one opening 204 allowing access to the therapy chamber 201, wherein the at least one opening 204 may be located between the two parts of the therapy chamber 201.

A method of pressure therapy may comprise:
a) sealing the at least part of the body of the subject of the therapy by at least one sealing unit 202;
b) providing at least one hypobaric period and/or at least one hyperbaric period in a therapy unit 105 to at least part of the body of a subject of the therapy, wherein the at least one hypobaric period and/or at least one hyperbaric period is provided by at least one pressure generator 106 connected to the therapy unit 105;
c) controlling the pressure in the therapy unit 105 and the course of the therapy by at least one control unit 103;
d) monitoring the at least one parameter of the therapy by at least one sensor 107.

The method of pressure therapy may comprise providing an alternating pressure in the therapy unit 105 by the pressure generator 106, wherein a first period of non-atmospheric pressure is followed by a second period of non-atmospheric pressure.

The method of pressure therapy may comprise the first period of non-atmospheric pressure, that is the hyperbaric period and the second period of the non-atmospheric pressure, that is hypobaric period.

The method may comprise providing a thermal treatment.

The method may comprise providing airtight seal around the body part of the therapy subject by the sealing unit 202, wherein the pressure in the sealing unit 202 may be changed based on the pressure in the therapy unit 105.

The pressure in the sealing unit 202 may be adjusted based on a target pressure in the therapy unit 105 or based on the positive pressure threshold and/or the negative pressure threshold.

The pressure in the sealing unit 202 may be reduced during transitions of pressure in the therapy unit 105 from hyperbaric to hypobaric pressure.

The method of pressure therapy may comprise providing an alternating pressure cycle by the pressure generator 106, the alternating pressure cycle may comprise three periods, wherein a first period of non-atmospheric pressure may be followed by a second period of atmospheric pressure and a third period of non-atmospheric pressure.

1. A device for pressure therapy, the device comprising:
a pressure generator configured to generate positive or negative pressure;
a therapy unit connected to the pressure generator, the therapy unit comprising:
a therapy chamber configured to receive a body part; and
a sealing unit configured to seal the body part in the therapy chamber during the pressure therapy,
wherein the therapy chamber is configured to receive the body part via the sealing unit, and
wherein the sealing unit comprises:
a moldable membrane;
an adjustable aperture formed by the moldable membrane; and
actuators configured to exert force on the moldable membrane,
wherein the actuators are configured to move the moldable membrane in order to adjust the size of the adjustable aperture by a mechanical deformation of the moldable membrane; and
a control unit configured to control the pressure generator in order to provide the positive or negative pressure to the therapy unit during the pressure therapy, and to control the actuators in order to adjust the adjustable aperture.

2. The device of claim 1, wherein the moldable membrane comprises at least one of: a straight-line profile, an S-shaped profile, an O-shaped profile, or a C-shaped profile.

3. The device of claim 2, wherein the moldable membrane comprises a facet and a body, wherein a width of the facet is greater than a width of the body, the width being determined in a cross-section through the moldable membrane.

4. The device of claim 2, wherein the moldable membrane comprises at least one of: polymer material, silicone, natural latex, organic latex, synthetic latex, blended latex, neoprene, rubber, or polyurethane.

5. The device of claim 1, wherein the actuators comprise a first actuator selected from a hydraulic actuator, pneumatic actuator, or electric actuator.

6. The device of claim 5, wherein the first actuator is selected from a piston, motor, servo motor, linear actuator, or rotary actuator.

7. The device of claim 5, wherein the actuators are arranged in a circular configuration around the aperture.

8. The device of claim 5, wherein at least one actuator is positioned on one side of the moldable membrane and at least one actuator is positioned on an opposite side of the moldable membrane.

9. The device of claim 5, wherein the number of actuators is in a range from 4 to 50.

10. A device for pressure therapy, the device comprising:
a main unit comprising:
a pressure generator configured to generate:
a positive pressure higher than atmospheric pressure in a range of 0.1 kPa to 100 kPa, and
a negative pressure lower than atmospheric pressure in a range of 0.1 kPa to 100 kPa; and
a control unit configured to control the pressure generator; and
a therapy unit connected to the pressure generator, the therapy unit comprising:
a rigid therapy chamber configured to receive a body part and configured to withstand the positive pressure and the negative pressure, wherein the therapy chamber is adapted so that the body part is insertable into the therapy chamber; and
a sealing unit comprising a moldable membrane and a plurality of between 4 and 50 actuators, the actuators being configured to move the moldable membrane,
wherein the moldable membrane is configured to seal the body part in the therapy chamber during the pressure therapy;
wherein the control unit is configured to control the pressure generator in order to provide the negative pressure and the positive pressure to the therapy unit during the pressure therapy and to control the actuators in order to adjust an aperture formed by the moldable membrane.

11. The device of claim 10, wherein the moldable membrane has at least one of the following profiles: a straight-line profile, an S-shaped profile, an O-shaped profile, or a C-shaped profile,
wherein the moldable membrane comprises a facet and a body, wherein a width of the facet is greater than a width of the body, and
wherein the moldable membrane comprises at least one of: a polymer material, silicone, latex, neoprene, rubber, or polyurethane.

12. The device of claim 10, further comprising an opening configured to allow access to the therapy chamber, wherein the opening is located on a side of the therapy chamber, spaced apart from the sealing unit.

13. The device of claim 10, further comprising a positioning mechanism configured to adjust a position of the therapy unit relative to the main unit,
wherein the positioning mechanism is configured to provide at least one of: lifting, tilting, rotating, elevating, or lowering the therapy unit relative to the main unit.

14. The device of claim 13, wherein the positioning mechanism is configured to set an angle between the therapy unit and the main unit in a range from 0° to 180°.

15. The device of claim 13, further comprising a parking system for parking the device, the parking system comprising at least one wheel and a central brake.

16. The device of claim 15, further comprising an upper part and a bottom part, wherein the upper part comprises a user interface, the positioning mechanism, and the therapy unit, and wherein the bottom part comprises the parking system and the pressure generator.

17. A device for pressure therapy, the device comprising:
a main unit comprising:
a pressure generator configured to generate non-atmospheric pressure including a positive pressure higher than atmospheric pressure or a negative pressure lower than atmospheric pressure; and
a control unit configured to control the pressure generator;
a therapy unit connected to the pressure generator via a fluid conduit, the therapy unit comprising:
a therapy chamber configured to receive a body part, wherein the therapy chamber is adapted so that the body part is insertable into the therapy chamber; and
a sealing unit configured to seal the body part in the therapy chamber during the pressure therapy, the sealing unit comprising:
a moldable membrane comprising at least one of: polymer material, silicone, latex, natural latex, organic latex, synthetic latex, blended latex, neoprene, rubber, or polyurethane; and
a plurality of actuators configured to move the moldable membrane; and
a user interface configured to provide a setup of the therapy or one or more therapy protocols to a user of the device,
wherein the control unit is configured to control the pressure generator in order to provide the negative pressure or the positive pressure to the therapy unit during the pressure therapy according to the setup or the one or more therapy protocols and to control the plurality of actuators.

18. The device of claim 17, wherein the user interface is a front-mounted user interface, and wherein the therapy unit is positioned between at least a portion of the main unit and the user interface.

19. The device of claim 17, wherein the control unit is configured to provide an alternating pressure in the therapy unit by the pressure generator, wherein a first period of non-atmospheric pressure is followed by a second period of non-atmospheric pressure different from the first period of non-atmospheric pressure.

20. The device of claim 17, wherein the therapy protocol comprises a first period of non-atmospheric pressure followed by a second period of atmospheric pressure, followed by a third period of non-atmospheric pressure.

21. The device of claim 20, wherein the first period of non-atmospheric pressure comprises either positive pressure or negative pressure, the first period having a pressure difference between the pressure in the therapy chamber and the atmospheric pressure in a range of 0.1 kPa to 100 kPa;
wherein the third period of non-atmospheric pressure comprises a pressure opposite in polarity to the pressure of the first period, the third period having a pressure difference between the pressure in the therapy chamber and the atmospheric pressure in a range of 0.1 kPa to 100 kPa.

22. The device of claim 21, wherein the first period of non-atmospheric pressure has a duration in a range of 1 second to 100 seconds,
wherein the second period of atmospheric pressure has a duration in a range of 1 second to 20 seconds, and
wherein the third period of non-atmospheric pressure has a duration in a range of 1 second to 100 seconds.

23. The device of claim 22, wherein a pressure provided by the sealing unit on the body part to seal the body part is reduced during the second period of atmospheric pressure.

24. A device for pressure therapy, the device comprising:
a pressure generator configured to generate positive or negative pressure;
a therapy unit connected to the pressure generator, the therapy unit comprising:
a therapy chamber configured to receive a body part, wherein the therapy chamber is adapted so that the body part is insertable into the therapy chamber; and
a sealing unit configured to seal the body part in the therapy chamber during the pressure therapy,
wherein the sealing unit comprises a moldable membrane, an actuator, and an aperture; and
a control unit configured to control the pressure generator in order to provide the negative pressure or the positive pressure to the therapy unit during the pressure therapy, and to control the actuator.

25. The device of claim 24, wherein the actuator is hydraulic, pneumatic, or electric and comprises at least one of: a piston, a motor, a servo motor, a linear actuator, or a rotary actuator; and
wherein the aperture has an adjustable size.

26. The device of claim 25, further comprising a wrap configured to be applied to a portion of the body part that is in contact with the sealing unit during the pressure therapy.

27. The device of claim 25, further comprising at least one sensor and at least one fluid conduit, wherein the pressure generator is connected to the therapy unit by the at least one fluid conduit.

28. The device of claim 27, wherein the control unit is further configured to control the sealing unit to provide an airtight seal around the body part, and wherein a pressure provided by the sealing unit on the body part is changed based on the pressure in the therapy unit.

29. The device of claim 25, wherein the therapy chamber has a length in a range from 1 cm to 250 cm and a width in a range from 1 cm to 100 cm.

30. The device of claim 29, wherein the therapy unit comprises at least one opening comprising at least one seal, and wherein the at least one opening is:
(i) located on a side of the therapy chamber;
(ii) spaced apart from the sealing unit; and
(iii) configured to allow access to the therapy chamber when unsealed.
